SEQUENCE LISTING (94 sequences)

SEQ ID NO: 1 - DNA, 28 bp, Artificial Sequence
  Chemically Synthesized
gcctcagctctcacctgcccatcttttg

SEQ ID NO: 2 - DNA, 27 bp, Artificial Sequence
  Chemically Synthesized
aggcagcatggaggctggtcagttgaa

SEQ ID NO: 3 - DNA, 32 bp, Artificial Sequence
  Chemically Synthesized
gacaagcgcttcgccttcatccgctcagacag

SEQ ID NO: 4 - DNA, 27 bp, Artificial Sequence
  Chemically Synthesized
agaaaaccagttctgctgactgggtga

SEQ ID NO: 5 - DNA, 27 bp, Artificial Sequence
  Chemically Synthesized
tggcttgggatttttatgggggtgctg

SEQ ID NO: 6 - DNA, 31 bp, Artificial Sequence
  Chemically Synthesized
catggttttagaaatcatcaagcctaggtca

SEQ ID NO: 7 - DNA, 27 bp, Artificial Sequence
  Chemically Synthesized
tatgctcaggtgtcctccaagaaatca

SEQ ID NO: 8 - DNA, 27 bp, Artificial Sequence
  Chemically Synthesized
ttgttgctccatatcctgtccctggag

SEQ ID NO: 9 - DNA, 33 bp, Artificial Sequence
  Chemically Synthesized
acatgtgctccacatttcagaacctatcttctt

SEQ ID NO: 10 - DNA, 29 bp, Artificial Sequence
  Chemically Synthesized
cctgacaatcgttgtgcagttgatgtcca

SEQ ID NO: 11 - DNA, 27 bp, Artificial Sequence
  Chemically Synthesized
cagcacctggtcttgcagggttgtgtg

SEQ ID NO: 12 - DNA, 36 bp, Artificial Sequence
  Chemically Synthesized
gtgtgggtctctaccttgggtgctgttctctgcctc

SEQ ID NO: 13 - DNA, 20 bp, Artificial Sequence
  Chemically Synthesized
acatcagggaaaagccattg

SEQ ID NO: 14 - DNA, 20 bp, Artificial Sequence
  Chemically Synthesized
tgggaatgggcactatgatt

SEQ ID NO: 15 - DNA, 25 bp, Artificial Sequence
  Chemically Synthesized
gattggggacatgcagagtccaagg

SEQ ID NO: 16 - DNA, 27 bp, Artificial Sequence
  Chemically Synthesized
gcccctagcacccgacaagctgagtgt

SEQ ID NO: 17 - DNA, 26 bp, Artificial Sequence
  Chemically Synthesized
ccaggcccatgaccagatcagcacag

SEQ ID NO: 18 - DNA, 30 bp, Artificial Sequence
  Chemically Synthesized
agccatgcgccggaccacgacgtcacgcag

SEQ ID NO: 19 - DNA, 27 bp, Artificial Sequence
  Chemically Synthesized
gcccctagcacccgacaagctgagtgt

SEQ ID NO: 20 - DNA, 26 bp, Artificial Sequence
  Chemically Synthesized
ccaggcccatgaccagatcagcacag

SEQ ID NO: 21 - DNA, 30 bp, Artificial Sequence
  Chemically Synthesized
aacggcagcgccttcttgctggcacccaat

SEQ ID NO: 22 - DNA, 27 bp, Artificial Sequence
  Chemically Synthesized
aagcgtcgctactcctcccccaagagc

SEQ ID NO: 23 - DNA, 27 bp, Artificial Sequence
  Chemically Synthesized
gtcacacacagcacgtccaccgaggtc

SEQ ID NO: 24 - skipped

SEQ ID NO: 25 - DNA, 34 bp, Artificial Sequence
  Chemically Synthesized
gggaggcaacctgctggtcatcgtggccatcgcc

SEQ ID NO: 26 - DNA, 27 bp, Artificial Sequence
  Chemically Synthesized
tgttcttgtgcaaaggcaatgctaccg

SEQ ID NO: 27 - DNA, 27 bp, Artificial Sequence
  Chemically Synthesized
tcttaccctgagttcagttccgtctgc

SEQ ID NO: 28 - DNA, 33 bp, Artificial Sequence
  Chemically Synthesized
gaaggaaataaattcacagtcaaagaatcaagc

SEQ ID NO: 29 - DNA, 30 bp, Artificial Sequence
  Chemically Synthesized
ggagactggcaaagcagagtttttgcgaga

SEQ ID NO: 30 - DNA, 29 bp, Artificial Sequence
  Chemically Synthesized
ggagacatgcttgccctgctaggttggtc

SEQ ID NO: 31 - DNA, 31 bp, Artificial Sequence
  Chemically Synthesized
ctgatactgactcttaccaaacaaagcatga

SEQ ID NO: 32 - DNA, 29 bp, Artificial Sequence
  Chemically Synthesized
tgtgggtactggacacagacaggtgttcc

SEQ ID NO: 33 - DNA, 29 bp, Artificial Sequence
  Chemically Synthesized
tcattaattgtttggctcaatgggtcatc

SEQ ID NO: 34 - DNA, 33 bp, Artificial Sequence
  Chemically Synthesized
ggcagagataatagaaggaatcatagtgtcatc

SEQ ID NO: 35 - DNA, 29 bp, Artificial Sequence
  Chemically Synthesized
tgtggcaatagccaagaacaagaatctgc

SEQ ID NO: 36 - DNA, 29 bp, Artificial Sequence
  Chemically Synthesized
gtccactgcaattgaaagcaggctgcaaa

SEQ ID NO: 37 - DNA, 30 bp, Artificial Sequence
  Chemically Synthesized
ccgtatctgtactgtttaatagggtgatga

SEQ ID NO: 38 - DNA, 29 bp, Artificial Sequence
  Chemically Synthesized
cccatgggagatcaatcttttcttcagat

SEQ ID NO: 39 - DNA, 30 bp, Artificial Sequence
  Chemically Synthesized
gctcatcatcaatatcagagccagagtgtg

SEQ ID NO: 40 - DNA, 33 bp, Artificial Sequence
  Chemically Synthesized
ggttacttagttacgaagccaataccaacctat

SEQ ID NO: 41 - DNA, 27 bp, Artificial Sequence
  Chemically Synthesized
tgccagccaattcaagcccagtccttt

SEQ ID NO: 42 - DNA, 31 bp, Artificial Sequence
  Chemically Synthesized
acacaacctggaagacaaactacaagagcaa

SEQ ID NO: 43 - DNA, 31 bp, Artificial Sequence
  Chemically Synthesized
gacagtgtatcagtgaaggaatcgctttctg

SEQ ID NO: 44 - DNA, 27 bp, Artificial Sequence
  Chemically Synthesized
ttagccaccccactcccagcttcatcc

SEQ ID NO: 45 - DNA, 27 bp, Artificial Sequence
  Chemically Synthesized
caggtgcatcgtgcacataagcctcgt

SEQ ID NO: 46 - DNA, 33 bp, Artificial Sequence
  Chemically Synthesized
gctcaggtgtcctccaagaaatcaaattttgcc

SEQ ID NO: 47 - DNA, 27 bp, Artificial Sequence
  Chemically Synthesized
tcccagttttgcagatgaggcaatgga

SEQ ID NO: 48 - DNA, 27 bp, Artificial Sequence
  Chemically Synthesized
aagccctggggaatgaggtggcaaaga

SEQ ID NO: 49 - DNA, 33 bp, Artificial Sequence
  Chemically Synthesized
gcaaatctaactcttcaagctaacacatagcaa

SEQ ID NO: 50 - DNA, 20 bp, Artificial Sequence
  Chemically Synthesized
aaatcagaaggctgcttgga

SEQ ID NO: 51 - DNA, 20 bp, Artificial Sequence
  Chemically Synthesized
atgggtgaatgggaatttga

SEQ ID NO: 52 - DNA, 32 bp, Artificial Sequence
  Chemically Synthesized
ctcaaatacttgcacagaggctcactcccttg

SEQ ID NO: 53 - DNA, 27 bp, Artificial Sequence
  Chemically Synthesized
aactgcgtgcaaccttcaatcctgctg

SEQ ID NO: 54 - DNA, 28 bp, Artificial Sequence
  Chemically Synthesized
tgtggggcaagggacaaagatgctatgg

SEQ ID NO: 55 - DNA, 27 bp, Artificial Sequence
  Chemically Synthesized
gaagaagggctcttttaataatcacac

SEQ ID NO: 56 - DNA, 20 bp, Artificial Sequence
  Chemically Synthesized
acaagttctgggggacacag

SEQ ID NO: 57 - DNA, 20 bp, Artificial Sequence
  Chemically Synthesized
aggccatgctgctgcagaca

SEQ ID NO: 58 - DNA, 33 bp, Artificial Sequence
  Chemically Synthesized
gaccttctatctgaggaacaaccaactagttgc

SEQ ID NO: 59 - DNA, 27 bp, Artificial Sequence
  Chemically Synthesized
tgagcaaatgtggctcctgggggttct

SEQ ID NO: 60 - DNA, 27 bp, Artificial Sequence
  Chemically Synthesized
cccaaagcctgtcaaggccaaggacat

SEQ ID NO: 61 - DNA, 31 bp, Artificial Sequence
  Chemically Synthesized
gatggctgtgcctctgcctgtctcccccacc

SEQ ID NO: 62 - DNA, 27 bp, Artificial Sequence
  Chemically Synthesized
cctggaggccccagcaggtgatgttta

SEQ ID NO: 63 - DNA, 27 bp, Artificial Sequence
  Chemically Synthesized
cctgcaggagcagctacccttgggaac

SEQ ID NO: 64 - DNA, 33 bp, Artificial Sequence
  Chemically Synthesized
ggccagcctgacctgggacctgtgcctcacctc

SEQ ID NO: 65 - DNA, 27 bp, Artificial Sequence
  Chemically Synthesized
caccccaaaacccagtggcttgaaaca

SEQ ID NO: 66 - DNA, 29 bp, Artificial Sequence
  Chemically Synthesized
tgcagtctcctctcacagggggctagact

SEQ ID NO: 67 - DNA, 35 bp, Artificial Sequence
  Chemically Synthesized
ggggtcactttggaagctgcattcagcagagtgcc

SEQ ID NO: 68 - DNA, 27 bp, Artificial Sequence
  Chemically Synthesized
ggcattacctgcagcaagggcctgtgt

SEQ ID NO: 69 - DNA, 27 bp, Artificial Sequence
  Chemically Synthesized
gccgtgacattgtccacaaggccagat

SEQ ID NO: 70 - DNA, 31 bp, Artificial Sequence
  Chemically Synthesized
gagccctaagtctaagatagggcagatagca

SEQ ID NO: 71 - DNA, 584 bp, Homo sapiens
  Allele (329)..(329): rs1042714; s at position 329 may be either C or G
ctagtctgcgcacataacggtgcagaacgcactcgcgaagcggcttcttcagagcacgggctggaactggcaggcaccgcgagcccctagcacccgacaagctgagtgtgcaggacgagtccccaccacacccacaccacagccgctgaatgaggcttccaggcgtccgctcgcggcccgcagagccccgccgtgggtccgcccgctgaggcgcccccagccagtgcgctcacctgccagactgcgcgccatggggcaacccgggaacggcagcgccttcttgctggcacccaatggaagccatgcgccggaccacgacgtcacgcagsaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttcca

SEQ ID NO: 72 - DNA, 551 bp, Homo sapiens
  Allele (296)..(296): rs1042713; r at position 296 may be either G or A
ctagtctgcgcacataacggtgcagaacgcactcgcgaagcggcttcttcagagcacgggctggaactggcaggcaccgcgagcccctagcacccgacaagctgagtgtgcaggacgagtccccaccacacccacaccacagccgctgaatgaggcttccaggcgtccgctcgcggcccgcagagccccgccgtgggtccgcccgctgaggcgcccccagccagtgcgctcacctgccagactgcgcgccatggggcaacccgggaacggcagcgccttcttgctggcacccaatrgaagccatgcgccggaccacgacgtcacgcaggaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggactt

SEQ ID NO: 73 - DNA, 511 bp, Homo sapiens
  Allele (256)..(256): rs4994; y at position 256 may be either C or T
tgagccaggtgatttgggagaccccctccttccttctttccctaccgccccacgcgcgacccggggatggctccgtggcctcacgagaacagctctcttgccccatggccggacctccccaccctggcgcccaataccgccaacaccagtgggctgccaggggttccgtgggaggcggccctagccggggccctgctggcgctggcggtgctggccaccgtgggaggcaacctgctggtcatcgtggccatcgccyggactccgagactccagaccatgaccaacgtgttcgtgacttcgctggccgcagccgacctggtgatgggactcctggtggtgccgccggcggccaccttggcgctgactggccactggccgttgggcgccactggctgcgagctgtggacctcggtggacgtgctgtgtgtgaccgccagcatcgaaaccctgtgcgccctggccgtggaccgctacctggctgtgaccaacccgctgcgttacggcgcactgg

SEQ ID NO: 74 - DNA, 601 bp, Homo sapiens
  Allele (301)..(301): rs1799883, r at position 301 may be either G or A
aagttatggaaaaacaactttaatcagttctcttgatcggattgaacctgaacttctgtagaagcaatctgaatgttcttgtgcaaaggcaatgctaccgagttttcttcccaccctcaaaataaacaaacaaaacataacttggaaaaataaacacttcctatgggatttgactttattttctccattgtcttaccttttacaggtgttaatatagtgaaaaggaagcttgcagctcatgacaatttgaagctgacaattacacaagaaggaaataaattcacagtcaaagaatcaagcrcttttcgaaacattgaagttgtttttgaacttggtgtcacctttaattacaatctagcagacggaactgaactcagggtaagaatttttttttttatgagcaatgcattcttgatttttctacccaatattaaaatgatttctgctctatttcattggatggtttaattaatgcaggtctccttcactaactgaagaagccaatgaagtttgtctacattatatattgcacaaattggcaggatatttaaatatgtttttatttttatacgcatctgtgaagaatctgaattgaacagta

SEQ ID NO: 75 - DNA, 701 bp, Homo sapiens
  Allele (501)..(501): rs10496444, y at position 501 may be either C or T
ccagggccaggcacagtggcccacgcctgtaatcccagcactttgggaggccaaggtgggtggatcacctgaagtcaggagttcaagaccaacctgatcaacatggtgaaaccccatctctactaaaaatacaaaattagctgggcatggtggtgcacacctgtaatcccagctacttgggaggctgaggcaggagaatcatttgaacctgggaggtagaggttgcagtgagcagagatcgtgccactgcactccagcctgggtgacagggagactccgtctcaaaaaaaaaaaaacaaaaaaaaaaccaaaaaaaaaacaaaaaacaagaattactatcccagttttgcagatgaggcaatggaagctctaaaaagttaagtaggagaaacaaacatgaaatgtatgtcttatgcttttcctcatcctatttcctcagcctggaatgtccattctccctccactatgcaaatctaactcttcaagctaacacatagcaaygtctgagaaaccgtccctgtgttcactctgttagcctcacttgctccctccccatccctctgtttcctttctgttataacacttctctattctgctggcatcacagtcatctccacctgccttcctcacaagttaaaagcttgttaagggcaagtggtgttctttgccacctcattccccagggcttctaacacagtgcc

SEQ ID NO: 76 - DNA, 511 bp, Homo sapiens
  Allele (256)..(256): rs17561, k at position 256 may be either G or T
atgaaactattgcctgaggaattgaagtttaaaaagaagcacataagcaacaacaaggataatcctagaaaaccagttctgctgactgggtgatttcacttctctttgcttcctcatctggattggaatattcctaatatcccctccagaactattttccctgtttgtactagactgtgtatatcatctgtgtttgtacatagacattaatctgcacttgtgatcatggttttagaaatcatcaagcctaggtcakcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcaggtacattaaaatggcaccagacatttctgtcatcctcccctcctttcatttacttatttatttatttcaatctttctgcttgcaaaaaacatacctcttcagagt

SEQ ID NO: 77 - DNA, 800 bp, Homo sapiens
  Allele (401)..(401): rs1143633, r at position 401 may be either G or A
gccaaagccctttaaaggtagaaggcccagcggggagacaaaacaaagaaggctggaaaccaaagcaatcatctctttagtggaaactattcttaaagaagatcttgatggctactgacatttgcaactccctcactctttctcaggggcctttcacttacattgtcaccagaggttcgtaacctccctgtgggctagtgttatgaccatcaccattttacctaagtagctctgttgctcggccacagtgagcagtaatagacctgaagctggaacccatgtctaatagtgtcaggtccagtgttcttagccaccccactcccagcttcatccctactggtgttgtcatcagactttgaccgtatatgctcaggtgtcctccaagaaatcaaattttgccrcctcgcctcacgaggcctgcccttctgattttatacctaaacaacatgtgctccacatttcagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaaggtaaatggaaactcctggtttccctgcctggcctcctggcagcttgctaattctccatgttttaaacaaagtagaaagttaatttaaggcaaatgatcaacacaagtgaaaaaaaatattaaaaaggaatatacaaactttggtcctagaaatggcacatttgattgcact

SEQ ID NO: 78 - DNA, 799 bp, Homo sapiens
  Allele (400)..(400): rs1143643, r at position 400 may be either G or A
ccttcaatcctgctgcagaaaattaaatcattttgccgatgttattatgtcctaccatagttacaaccccaacagattatatattgttagggctgctctcatttgatagacaccttgggaaatagatgacttaaagggtcccattatcatgtccactccactcccaaaattaccaccactatcacctccagctttctcagcaaaagcttcatttccaagttgatgtcattctaggaccataaggaaaaatacaataaaaagcccctggaaactaggtacttcaagaagctctagcttaatttcacccccccaaaaaaaaaaaattctcacctacattatgctcctcagcatttggcactaagttttagaaaagaagaagggctcttttaataatcacacrgaaagttgggggcccagttacaactcaggagtctggctcctgatcatgtgacctgctcgtcagtttcctttctggccaacccaaagaacatctttcccatagcatctttgtcccttgccccacaaaaattcttctttctctttcgctgcagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttctgggagggaccaaaggcggccaggatataactgacttcaccatgcaatttgtgtcttcctaaagagagctgtacccagagagtcctgtgctgaatgtg

SEQ ID NO: 79 - DNA, 801 bp, Homo sapiens
  Allele (401)..(401): rs1143634, y at position 401 may be either C or T
tagtggaaactattcttaaagaagatcttgatggctactgacatttgcaactccctcactctttctcaggggcctttcacttacattgtcaccagaggttcgtaacctccctgtgggctagtgttatgaccatcaccattttacctaagtagctctgttgctcggccacagtgagcagtaatagacctgaagctggaacccatgtctaatagtgtcaggtccagtgttcttagccaccccactcccagcttcatccctactggtgttgtcatcagactttgaccgtatatgctcaggtgtcctccaagaaatcaaattttgccgcctcgcctcacgaggcctgcccttctgattttatacctaaacaacatgtgctccacatttcagaacctatcttcttygacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaaggtaaatggaaacatcctggtttccctgcctggcctcctggcagcttgctaattctccatgttttaaacaaagtagaaagttaatttaaggcaaatgatcaacacaagtgaaaaaaaatattaaaaaggaatatacaaactttggtcctagaaatggcacatttgattgcactggccagtgcatttgttaacaggagtgtgaccctgagaaattagacggctcaagcactcccaggaccatgtccaccca

SEQ ID NO: 80 - DNA, 701 bp, Homo sapiens
  Allele (201)..(201): rs1143623, s at position 201 may be either C or G
aacagcttttagatttcctgtggaaaatataacttactaaagatggagttcttgtgactgactcctgatatcaagatactgggagccaaattaaaaatcagaaggctgcttggagagcaagtccatgaaatgctctttttcccacagtagaacctatttccctcgtgtctcaaatacttgcacagaggctcactcccttgsataatgcagagcgagcacgatacctggcacatactaatttgaataaaaatgctgtcaaattcccattcacccattcaagcagcaaactctaccacctgaatgtacatgccaggcactgtgctagacttggctcaaaaagatttcagtttcctggaggaaccaggaggagcaaggtttcaactcagtgctataagaagtgttacaggctggacacggtggctcacgcctgtaatcccaacactttgggaggccgaggcgggcagatcacaaggtcaggagatcgagaccatcctggctaacatggtgaaaccctgtctctactaaaaatacaaaaaattagccgggcgtggcggcaggtgcctgtagtcccagctgctggggaggctgaggcaggagaatggtgtgaacccgggaggcggaacttgcagggggccgagatcgtgccactgcactccagcctgggcgacagagtgagactctgtctcaaaaaaaaaaaaaaa

SEQ ID NO: 81 - DNA, 601 bp, Homo sapiens
  Allele (301)..(301): rs4848306, r at position 301 may be either G or A; on complementary strand y may be either C or T
tttccccaatttcccatctcattcttggcactggcttcctaatatttgttcttatgagtcattttcttgtatcatttccatgagtccctctgggatcttaaagtatgaaaaatgttgtgtgtacccacacctgtctttgtggatatttctctcctttcccttctgcttctgggattatttgggaatgggcactatgatttttatcatatcgcttccacttcctttatggcatcatctccaatgggcttcttctccctcttggatccaggttctcagattggggacatgcagagtccaaggracattccattctcctccctggtctagaacaaggagggcttagatatatgagcaggtggctggggctggcgagctatgtagtctccaatggcttttccctgatgtcggagttgttatgtcagttctgggagaccaataagaccttgtctttcctttggatccatcagaaaaagcccctgggtgggtaagatggatggcagggctctcctactctatgtcttttctcacacctagtgggtataagagaggggaccacaaacagagggggctctggtaccacttatccagggtctggaaacat

SEQ ID NO: 82 - DNA, 599 bp, Homo sapiens
  Allele (300)..(300): rs16944, r at position 300 may be either G or A; on complementary strand y may be either C or T
tgcaaatgtatcaccatgcaaatatgcattgttttcctgacaatcgttgtgcagttgatgtccacattaaaatactggattttcccacgttagaagaatgtttaaatttagtatatgtgggacaaagtggaagacacacagatttatacatgcacatacttttcttcattcacttctttgtacttaagtttaggaatcttcccacttacagatggataaatgggtacaatgaagggccaaagccctccctgtctgtattgagggtgtgggtctctaccttgggtgctgttctctgcctcrggagctctctgtcaattgcaggagcctctgaggagaaaattgacctttcttggctggggcagagaacatacggtatgcagggttcaggctcctgacggagttggggcaaccctggagatagctcacacaaccctgcaagaccaggtgctgttaccctagccaatctcatggatgaaccagatcaatgccagatgagctctgcctaaaatgattttttggtgaactctgaaaagtggaatattgtttctgtaagaatatccatctgagactctatctcttggtaataccaagagttatca

SEQ ID NO: 83 - DNA, 601 bp, Homo sapiens
  Allele (301)..(301): rs1794066, r at position 301 may be either G or A
ggctctgtgggtgtatgagtgacaagtctctcccttccagatatggggactgtctgcttccctaggttgcctctccctgctctgatcagctagaagctccaggagatcctcctggaggccccagcaggtgatgtttatccctccagactgaggctaaatctagaaactaggataatcacaaacaggccaatgctgccatatgcaaagcactttggtttgcctggccacccctcgtcgagcatgtgggctcttcagagccacctgatgaggtgggtacagttagccacacttcacaggtgargaggtgaggcacaggtcccaggtcaggctggccggagctctgtttattacgtctcacagctttgagtcctgctctcaaccagagaggccctttaccaagaagaaaggattgggacccagaatcaggtcactggctgaggtagagaggaagccgggttgttcccaagggtagctgctcctgcaggactctgagcaggtcaccagctaatggaggaaaggctctagggaaagacccttctggtctcagactcagagcgagttagctgcaaggtgttccgtctcttgaaacttctacctaggt

SEQ ID NO: 84 - DNA, 842 bp, Homo sapiens
  Allele (345)..(345): rs380092, w at position 345 may be either A or T
cggggtctcttcattattgctgcttcctcttctattaacctgaccctcccctctgttcttccccagaaaagatagatgtggtacccattgagcctcatgctctgttcttgggaatccatggagggaagatgtgcctgtcctgtgtcaagtctggtgatgagaccagactccagctggaggtaaaaacatgctttggatctcaaatcaccccaaaacccagtggcttgaaacaaccaaaattttttcttatgattctgtgggttgaccaggattagctgggtagttctgttccatgtggtggaacatgctggggtcactttggaagctgcattcagcagagtgccwggcttgcgctgggcatccaaggtggtccctcatcctccaggctctctttccatgtgatctctcagtgtttaagagttagttggagcttccttacagcatggcggctgacttccaaaagggattattccaaaaagagcctcaacatgcaggcgcttattatgacttctgcttgcatcatcctattggccaaagccagtcacgtggctaagtctagccccctgtgagaggagactgcataagagtgtgaacaccaggagacacggtcactgggggccaccactgtaaccatctaccacaggacctgaatctctgtgtgctactcccttgctcaagggcccccctacccacgcagacctgctgtcttctagcaaagcccatcctcaggacctttctcttccaatccttattgactcaaattgattagttggtgctccacccagagccctgtgctcctttatctcatgtaatgttaatgggtttcccagccctgggaaaac

SEQ ID NO: 85 - DNA, 511 bp, Homo sapiens
  Allele (256)..(256): rs4251961, y at position 256 may be either C or T
tgttgagaagtttatgaattatgagcccttctgcaaatgagagggttcttccctgtcaggagggacagattgtaggtggcaagattggtggcagccagtaggctggtctgctccttcctctctatttcatatgtgtatgaaggcattacctgcagcaagggcctgtgtaaatgcatgtgatttacagagcattttatgtactgcgtgtcattcatgcttccggtgagccctaagtctaagatagggcagatagcaycaggtccattttgcagctgtcaaaatgaggtctgaagggcagaagtggtgtgcccacacacacacaactggttggctgcagacctggggactagacccgggacttcgtcctgcccaggggtctcttgccactgctccccatcaacttggatggctttaagcatttgtgagttgtctgctccctgatggcagaatgcagagacatgaagctacaagcaggttcgctcccaacggcaaaaaggaggaggggtgttca

SEQ ID NO: 86 - DNA, 701 bp, Homo sapiens
  Allele (501)..(501): rs419598, y at position 501 may be either C or T
aatagaagaaaatttaaaatccagacccttggtcacactatccacatttaaagaggtcaatagccacatgtggttagtggccaccctattgggcagtgcagctacagaacatttttgcatcccagaaagttcttttggatgttgctgctctacagcatgctttgctgaaacagaagtgccttccctgggaatctcagatgggaagcaagtaaggaggggagtcaaatgtgggctcactgctcaccagctgtgagggttgggcctgcctcttaaccattgtcagcctcagtcttctcatccatgcatgccgtgggtatactaaaatactatacccctggaagagctggatgcaaatttgacaagttctgggggacacaggaaggtgccaagcacaaggctgggcacatggtggctgtgcactacagctgagtccttttccttttcagaatctgggatgttaaccagaagaccttctatctgaggaacaaccaactagttgcyggatacttgcaaggaccaaatgtcaatttagaaggtgagtggttgccaggaaagccaatgtatgtgggcatcacgtcactttgcccgtctgtctgcagcagcatggcctgcctgcacaaaccctaggtgcaatgtcctaatccttgttgggtctttgtattcaagtttgaagctgggagggcctggctactgaagggcac

SEQ ID NO: 87 - DNA, 601 bp, Homo sapiens
  Allele (301)..(301): rs315952, y at position 301 may be either C or T
gacagagcaagactccgtctcgggaaaattaattaataaataaataaacctaggtcccagagtcccacagaatggcagacaggagcacctgggggcttttagggtatggcatttcccctgtactaactctgggctgtccagagggccatttcatggcgtggagtggagagggaggcagcacaggacttcctaggcctcagctctcacctgcccatcttttgatttccaggcagttaacatcactgacctgagcgagaacagaaagcaggacaagcgcttcgccttcatccgctcagacagyggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaagaacctggtcacaggactc

SEQ ID NO: 88 - DNA, 619 bp, Homo sapiens
  Allele (277)..(277): rs9005, r at position 277 may be either G or A
ttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccrggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctatattgttgactatgtccttggccttgacaggctttgggtatagagtgctgaggaaactgaaagaccaatgtgtctttcttaccccagaggctggcgcctggcctcttctctgagagttcttttcttccttcagcctcactctccctggataacatgagagc

SEQ ID NO: 89 - DNA, 601 bp, Homo sapiens
  Allele (301)..(301): rs12970134, r at position 301 may be either G or A
ctggtttctaaaatagttccatgctggtcaggtcattggaagaggcttcctgttcttttataaacattctaacatggagactggcaaagcagagtttttgcaagaaaagaactgattttaaaaaggtggcttcttagattgatgtgttttaactttgtctgctgaatctaaagaataaggagagatcatcgcaaagtaaatggaattggatatagttctctgaattacttggggttagtccagattatttcggttctaagcaacagatactgatactgactcttaccaaacaaagcatgarcaaacaaagatttatcagaagggtgcttgttagtacctgtattcaaagggagaactagtcaaacctcaaaggggcaaggccaaccaggaccaacctagcagggcaagcatgtctccacactgcctatcttcagatgagcatttttttcctttaggcaagtttttccaggtggtagtgggaatgaccaggagcagctcatgagctacatctgtgattcagagaggatgggactctttccccagctcagactcaaacactcctcgaagagactctgactggcttagctttggtcatatgtgc

SEQ ID NO: 90 - DNA, 601 bp, Homo sapiens
  Allele (301)..(301): rs2229616, r at position 301 may be either G or A
aactccacccaccgtgggatgcacacttctctgcacctctggaaccgcagcagttacagactgcacagcaatgccagtgagtcccttggaaaaggctactctgatggagggtgctacgagcaactttttgtctctcctgaggtgtttgtgactctgggtgtcatcagcttgttggagaatatcttagtgattgtggcaatagccaagaacaagaatctgcattcacccatgtactttttcatctgcagcttggctgtggctgatatgctggtgagcgtttcaaatggatcagaaaccattrtcatcaccctattaaacagtacagatacggatgcacagagtttcacagtgaatattgataatgtcattgactcggtgatctgtagctccttgcttgcatccatttgcagcctgctttcaattgcagtggacaggtactttactatcttctatgctctccagtaccataacattatgacagttaagcgggttgggatcatcataagttgtatctgggcagcttgcacggtttcaggcattttgttcatcatttactcagatagtagtgctgtcatcatctgcctcatcaccatgttcttca

SEQ ID NO: 91 - DNA, 801 bp, Homo sapiens
  Allele (401)..(401): rs4450508, r at position 401 may be either G or A
ggcggatcaagaggtcagaagatcgagaccatcctggctaacacggtgaaaccccgtctctactaaaaatacaaaaaaaattagccgggcgtggtggcgggcgcctgtagtcccagctactggagaggctgaggcaggagaatggcatgaacctgggaggcggagcttgcggtgagccgagattgcgccactgcactccagcctgggcgacaaagtgagactccgtctcaaaaaaaagaaaaaagaaagactaaatacacaccatcttctgcattgttacatgtggtgtcagaaattgttttttcatctttcatattcacttgctccatatagttaacatcatttcctcaaggaagaaaagaaagaaaaaaaagtggcagtacttcctttctgtggaacartggccccaattggtataggttatgttgcttagagcatgagacagacatgccgatgagaatatttatctgataagttgcttcctaattttgggtttatcagaccatcataattatattcattttctttgttaaatcaattcaaatctatattttttctttctttatagtcagcctcaggagatattgatttcacctttgatacaatagaactgaaaaattgtgactaacattattgctttggctaatggcagtgctttaaaatgtatggtttattctatgtgtaactaaaacactttacataatttgaaatataaatatttgctagatatcaacagattaattcttccagattatgattgtgtctgtcccctaatctctgcttgtcataattattgtattg

SEQ ID NO: 92 - DNA, 640 bp, Homo sapiens
  Allele (437)..(437): rs477181, k at position 437 may be either G or T
aattggttatagtgtgttaaaatagtagtatgaaagacaatagtattcttccattaaaaatattcattggggtttcactatttgtcagccattgtacagggtgatattaaaaccaagcttgatctctgctttccttgtgttaacagcttcatgagtgaagcagcattaatttaaaatgccgtgtgacacctgttaccatgaaaagttcatgccactattcatgcccatactgtgggtactggacacagacaggtgttcccagaaagatttttctgcaaaagtgaccctggagctgagttctgaaaaaggagaaggaattgactaagctaagtggaaagcaaaaagctttcagggcatcaggaacagcatggagcagagccctgaaagtaggccagcagactaaggcagagataatagaaggaatcatagtgtcatckgaggctgataattcatagtagagtattagtgtttgggagtgaaaagtctctatgtgaagtgatgtaatttgctgcaaattctgtggattacaatagcaacactaagtgcttaaaggagtgatatattagatgcactggtttttaaaaggactctgtctctgccagatgtactacattgcactgacgtctgctggatctagatg

SEQ ID NO: 93 - DNA, 601 bp, Homo sapiens
  Allele (301)..(301): rs502933, m at position 301 may be either C or A
tggtttttaaaaggactctgtctctgccagatgtactacattgcactgacgtctgctggatctagatgacccattgagccaaacaattaatgattttagttccgcaatacttggctcgttttttttccatgcaatgatctattatgtccactgagggctatagaatgttagagacagagaaattacaggcgtcccatgggagatcaatcttttcttcagatagagaaagtaaggttgagaggaagcggaaactttccatggttttgtggttacttagttacgaagccaataccaacctatmaattgattaaatgtaattctatagcttttgtccatcacacacaaatgatgattacaatgttaactgttttacatgattcattattcccagaagagtttgtttgttcacttttctgcagcctccaaaagaacatcatatgtgtgaaaacagattaacacactctggctctgatattgatgatgagctgttataatattaacaatgctgcatattgtggtggcagaaatccaaatgaacaagagattttggatatagcttgctgcaacacggaataaatgctttatacaaatgggaattctt

SEQ ID NO: 94 - DNA, 801 bp, Homo sapiens
  Allele (401)..(401): rs1801282, s at position 401 may be either G or C
tctgctctgataattctaaatacagtacagttcacgcccctcacaagacactgaacatgtgggtcaccggcgagacagtgtggcaatattttccctgtaatgtaccaagtcttgccaaagcagtgaacattatgacacaactttttgtcacagctggctcctaataggacagtgccagccaattcaagcccagtcctttctgtgtttattcccatctctcccaaatatttggaaactgatgtcttgactcatgggtgtattcacaaattctgttacttcaagtctttttcttttaacggattgatcttttgctagatagagacaaaatatcagtgtgaattacagcaaacccctattccatgctgttatgggtgaaactctgggagattctcctattgacscagaaagcgattccttcactgatacactgtctgcaaacatatcacaaggtaaagttccttccagatacggctattggggacgtgggggcatttatgtaagggtaaaattgctcttgtagtttgtcttccaggttgtgtttgttttaatactatcatgtgtacactccagtattttaatgcttagctcgttgctatcgcgttcatttaaaaacatgttcagaaccttaaaaaaggaaacctaacctaatctattttatctctgtgcatggctcccatttcctgaattttaagcattaaaggtatagttatatccaaaaacaatcctgttcatttttatttcctgagtttgcatagatttcccaagaatacataagggctttttagacttgaagggtcactt

(19) United States
(12) Patent Application Publication
(10) Pub. No.: US 2010/0112570 A1
(43) Pub. Date: May 6, 2010

(54) Genetic Markers for Weight Management and Methods of Use Thereof

(75) Inventors: Nazneen Aziz, Lexington, MA (US); Venkateswarlu Kondragunta, Woburn, MA (US); Prakash Prabhakar, Braintree, MA (US); Ken Kornman, Newton, MA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: INTERLEUKIN GENETICS, INC., Waltham, MA (US)

(21) Appl. No.: 12/466,602

(22) Filed: May 15, 2009

Related U.S. Application Data
(60) Provisional application No. 61/107,458, filed on Oct. 22, 2008.

(51) Int. Cl. C12Q 1/68

(52) U.S. Cl. 435/6

(57) ABSTRACT

This application relates to methods and tests that allow for the establishment of personalized weight-loss programs for a subject based upon the subject's metabolic genotype in key metabolic genes. Kits and methods are disclosed for determining a subject's metabolic genotype, which may be used to select an appropriate therapeutic/dietary regimen or lifestyle recommendation based upon the likelihood of a subject's responsiveness to certain diets and activity levels. Such a personalized weight-loss program will have obvious benefits (e.g., yield better results in terms of weight loss and weight maintenance) over traditional weight-loss programs that do not take into account genetic information.

### CROSS-REFERENCE TO RELATED APPLICATION

This application claims the benefit of the filing date of U.S. Provisional Patent Application No. 61/107,458, filed on Oct. 22, 2008, which is incorporated herein by reference in its entirety.

### BACKGROUND OF THE INVENTION

1. Field of the Invention

This application relates to methods of determining a subject's metabolic genotype and methods for selecting an appropriate therapeutic/dietary regimen or lifestyle recommendation based on subject's metabolic profile and susceptibility to adverse weight management issues.

2. Background

According to a report published in 1998 by the World Health Organization (WHO), obesity has reached epidemic proportions worldwide: about 1.7 billion people worldwide are overweight and 300 million of them are obese. In the U.S. approximately 127 million adults are overweight and 69 million are obese. Obese subjects are at increased risk of developing one or more serious medical conditions including diabetes, heart disease, high blood pressure and high blood cholesterol. The prevalence of obesity has more than doubled in the past 25 years and now reaches 31% among U.S. adults aged 20 years and older. Higher rates of obesity are seen among African-Americans and Hispanic Americans, especially among women (30% to 50%).

The increase in the prevalence of obesity observed worldwide in the past decades has occurred in a changing environment characterized by a progressive reduction of physical activity level and the abundance of highly palatable foods. The WHO Report identified these changes as the two principal modifiable characteristics of modern lifestyle promoting the development of obesity. However, despite the fact that people are exposed to the same environment, not everyone is becoming obese, suggesting a role for a subject's genetic profile in the development of weight management issues. That is, genetics determines a subject's susceptibility to become obese when exposed to a unfavorable environment as well as the way he/she can respond to diet and exercise.

Accordingly, there is a need for a means for establishing a personalized weight loss program that considers a person's genetic susceptibility to obesity in order to improve weight loss and weight maintenance outcomes relative to a similar program not taking into account genetic information. There is a need for a means for linking a subject's metabolic genotype to response to diet and/or exercise.

Genotype Screening

Traditional methods for the screening of heritable diseases have depended on either the identification of abnormal gene products (e.g., sickle cell anemia) or an abnormal phenotype (e.g., mental retardation). These methods are of limited utility for heritable diseases with late onset and no easily identifiable phenotypes such as, for example, vascular disease. With the development of simple and inexpensive genetic screening methodology, it is now possible to identify polymorphisms that indicate a propensity to develop disease, even when the disease is of polygenic origin. The number of diseases that can be screened by molecular biological methods continues to grow with increased understanding of the genetic basis of multifactorial disorders.

Genetic screening (also called genotyping or molecular screening), can be broadly defined as testing to determine if a patient has mutations (alleles or polymorphisms) that either cause a disease state or are “linked” to the mutation causing a disease state. Linkage refers to the phenomenon that DNA sequences which are close together in the genome have a tendency to be inherited together. Two sequences may be linked because of some selective advantage of co-inheritance. More typically, however, two polymorphic sequences are co-inherited because of the relative infrequency with which meiotic recombination events occur within the region between the two polymorphisms. The co-inherited polymorphic alleles are said to be in linkage disequilibrium with one another because, in a given human population, they tend to either both occur together or else not occur at all in any particular member of the population. Indeed, where multiple polymorphisms in a given chromosomal region are found to be in linkage disequilibrium with one another, they define a quasi-stable genetic “haplotype.” In contrast, recombination events occurring between two polymorphic loci cause them to become separated onto distinct homologous chromosomes. If meiotic recombination between two physically linked polymorphisms occurs frequently enough, the two polymorphisms will appear to segregate independently and are said to be in linkage equilibrium.

While the frequency of meiotic recombination between two markers is generally proportional to the physical distance between them on the chromosome, the occurrence of “hot spots” as well as regions of repressed chromosomal recombination can result in discrepancies between the physical and recombinational distance between two markers. Thus, in certain chromosomal regions, multiple polymorphic loci spanning a broad chromosomal domain may be in linkage disequilibrium with one another, and thereby define a broad-spanning genetic haplotype. Furthermore, where a disease-causing mutation is found within or in linkage with this haplotype, one or more polymorphic alleles of the haplotype can be used as a diagnostic or prognostic indicator of the likelihood of developing the disease. This association between otherwise benign polymorphisms and a disease-causing polymorphism occurs if the disease mutation arose in the recent past, so that sufficient time has not elapsed for equilibrium to be achieved through recombination events. Therefore identification of a human haplotype which spans or is linked to a disease-causing mutational change, serves as a predictive measure of a subject's likelihood of having inherited that disease-causing mutation. Importantly, such prognostic or diagnostic procedures can be utilized without necessitating the identification and isolation of the actual disease-causing lesion. This is significant because the precise determination of the molecular defect involved in a disease process can be difficult and laborious, especially in the case of multifactorial diseases such as inflammatory disorders.

Indeed, the statistical correlation between an obesity and a gene polymorphism does not necessarily indicate that the polymorphism directly causes the disorder. Rather the correlated polymorphism may be a benign allelic variant which is linked to (i.e. in linkage disequilibrium with) a disorder-causing mutation which has occurred in the recent human evolutionary past, so that sufficient time has not elapsed for equilibrium to be achieved through recombination events in the intervening chromosomal segment. Thus, for the purposes of diagnostic and prognostic assays for a particular disease, detection of a polymorphic allele associated with that disease can be utilized without consideration of whether the polymorphism is directly involved in the etiology of the disease. Furthermore, where a given benign polymorphic locus is in linkage disequilibrium with an apparent disease-causing polymorphic locus, still other polymorphic loci which are in linkage disequilibrium with the benign polymorphic locus are also likely to be in linkage disequilibrium with the disease-causing polymorphic locus. Thus these other polymorphic loci will also be prognostic or diagnostic of the likelihood of having inherited the disease-causing polymorphic locus. Indeed, a broad-spanning human haplotype (describing the typical pattern of co-inheritance of alleles of a set of linked polymorphic markers) can be targeted for diagnostic purposes once an association has been drawn between a particular disease or condition and a corresponding human haplotype. Thus, the determination of a subject's likelihood for developing a particular disease of condition can be made by characterizing one or more disease-associated polymorphic alleles (or even one or more disease-associated haplotypes) without necessarily determining or characterizing the causative genetic variation.

The description herein of disadvantages and problems associated with known methods is in no way intended to limit the scope of the embodiments described in this document to their exclusion.

### SUMMARY OF THE INVENTION

According to some embodiments of the present invention, the invention provides for methods and kits for determining a subject's metabolic genotype and selecting an appropriate therapeutic/dietary regimen or lifestyle recommendation for the subject. According to some embodiments, methods are provided for determining a subject's metabolic genotype, classifying the subject into one or more of a series of nutritional and exercise categories to which the subject is likely to be responsive, and communicating to the subject an appropriate therapeutic/dietary regimen or lifestyle recommendation for the subject. In this manner, a personalized weight-loss program may be chosen based on a subject's metabolic genotype. Such a personalized weight-loss program will have obvious benefits (e.g., yield better results in terms of weight loss and weight maintenance) over traditional weight-loss programs that do not take into account genetic information.

The invention provides a genetic predisposition test that allows predicting a subject's likely response to weight loss and weight management based on genetic polymorphisms in β-adrenergic receptors 2 and 3 (ADRB2 and ADRB3), peroxisome proliferation activator receptor-γ (PPARG), melanocortin receptor 4 (MCR4), fatty acid binding protein 2 (FABP2), and interleukin-1 (IL-1) pathway genes. The invention also provides kits to determine whether a subject is resistant to weight gain or weight loss based on analysis of genetic polymorphisms at these loci. This information can be used to screen subjects, such as obese and overweight subjects and classify them based on their genetic tendency to either lose weight or resistance to lose weight. Appropriate measure can then be implemented in life-style, diet, medicinal and possible surgical interventions. Such a genetic approach will help professionals in the field of weight-management to improve targeting patients with appropriate advise regarding their weight management.

According to some embodiments, methods are provided for predicting a subject's response to low calorie or calorie restricted diet for determining a subject's genotype with respect to any one, any two, any three, or any four, or all of the polymorphic loci or risk alleles selected from: IL1RN rs315952; IL1RN rs380092; IL1RN rs4251961; IL-1RN rs419598; IL-1RN 9005; IL1B rs1143633 (+3877); IL1B +6054; IL1B rs4848306 (−3737); IL1B rs1143623 (−1468); IL-1B rs1143634 (+3954); IL1B 16944 (−511); IL1A rs17561; ADRB2 rs1042713; ADRB2 rs1042714; ADRB3 rs4994; MCR-4 rs2229616; MCR-4 rs12970134; MCR-4 rs477181; MCR-4 rs502933; MCR-4 4450508; PPARG rs1801282 and FABP2 rs1799883, wherein the subject's genotype with respect to the loci provides information about the subject's likely response to low calorie diet or liquid diet, and allows the selection of a therapeutic/dietary regimen or lifestyle recommendation that is suitable to the subject's susceptibility to adverse weight management issues.

According to some embodiments of the invention, the low calorie diet comprises a diet of 1200-1500 kcal for women, and 1500-1800 kcal for men, and a liquid diet comprises of 1000 kcal for women and 1200 kcal for men.

According to some embodiments, methods are provided for selecting an appropriate therapeutic/dietary regimen or lifestyle recommendation for a subject by determining a subject's genotype with respect to any one, any two, any three, or any four, or all of the polymorphic loci selected from: IL1RN rs315952; IL1RN rs380092; IL1RN rs4251961; IL-1RN rs419598; IL-1RN 9005; IL1B rs1143633 (+3877); IL1B +6054; IL1B rs4848306 (−3737); IL1B rs1143623 (−1468); IL-1B rs1143634 (+3954); IL1B 16944 (−511); IL1A rs17561; ADRB2 rs1042713; ADRB2 rs1042714; ADRB3 rs4994; MCR-4 rs2229616; MCR-4 rs12970134; MCR-4 rs477181; MCR-4 rs502933; MCR-4 4450508; PPARG rs1801282 and FABP2 rs1799883, wherein the subject's genotype with respect to the loci provides information about the subject's likely response to low calorie diet or liquid diet, and allows the selection of a therapeutic/dietary regimen or lifestyle recommendation that is suitable to the subject's susceptibility to adverse weight management issues.

According to some embodiments, methods are provided for selecting therapeutic/surgical/dietary or lifestyle in overweight or obese subjects based on the identification in the subject's DNA one or more of the following alleles: ADRB2 (rs1042713; G), ADRB3 (rs4994; T), IL1A (rs17561; +4845; T), rs4848306 (−3737; C), rs1143623 (−1468; C) and rs16944 (−511; T) on IL1B gene and IL1RN (rs315952; C) loci, wherein the presence of any one, any two or all three genotypes will indicate that subject is predisposed to resistance to weight loss when prescribed a calorie restricted diet or low calorie liquid diet.

According to some embodiments, methods are provided for selecting therapeutic/surgical/dietary or lifestyle in overweight or obese subjects based on the identification in the subject's DNA one or more of the following alleles: ADRB2 (rs1042713; G), ADRB3 (rs4994; T), IL1A (rs17561; +4845; T), rs4848306 (−3737; C), rs1143623 (−1468; C) and rs16944 (−511; T) on IL1B gene and IL1RN (rs315952; C) loci, wherein the presence of any one, any two or all three genotypes will indicate that the subject is predisposed to resistance to weight loss when prescribed a calorie restricted diet or low calorie liquid diet.

According to some embodiments, methods are provided for selecting an appropriate therapeutic/dietary regimen or lifestyle recommendation for a subject by determining a subject's genotype with respect to any one, any two, any three, or any four, or all of the polymorphic loci from the following: IL1RN rs315952; IL1RN rs380092; IL1RN rs4251961; IL-1RN rs419598; IL-1RN 9005; IL1B rs1143633 (+3877); IL1B +6054; IL1B rs4848306 (−3737); IL1B rs1143623 (−1468); IL-1B rs1143634 (+3954); IL1B 16944 (−511); IL1A rs17561; ADRB2 rs1042713; ADRB2 rs1042714; ADRB3 rs4994; MCR-4 rs2229616; MCR-4 rs12970134; MCR-4 rs477181; MCR-4 rs502933; MCR-4 4450508; PPARG rs1801282 and FABP2 rs1799883; and classifying the subject's genotype into a nutrition responsiveness category and/or an exercise responsiveness category.

According to some embodiments, methods are provided for predicting a subject's response to a calorie restricted diet or low calorie liquid diet, wherein the subject is a carrier of the haplotype comprising a combination of the following markers: IL-1A +4845 (T), IL-1B +6054 (G), IL-1B +3877 (G), IL-1B +3954 (T), IL-1B −511 (C), IL-1B −3737 (C), IL-1B −511 (T), IL-1B −1468 (C), IL-1B +3954 (C), IL-1B −1468 (C), IL-1RN +2018 (T), IL-1RN 9005 (G), IL-1RN 315952 (C).

According to some embodiments, methods are provided for selecting an appropriate therapeutic/dietary regimen or lifestyle recommendation for a subject based on the identification in the subject's DNA one or more of the following allelic patterns, comprising any combination of the following IL-1 gene cluster haplotype markers: IL-1A +4845 (T), IL-1B +6054 (G), IL-1B +3877 (G), IL-1B +3954 (T), IL-1B −511 (C), IL-1B −3737 (C), IL-1B −511 (T), IL-1B −1468 (C), IL-1B +3954 (C), IL-1B −1468 (C), IL-1RN +2018 (T), IL-1RN 9005 (G), IL-1RN 315952 (C); wherein the presence of allelic pattern is predictive of the subject's response to diet and/or exercise and by selecting a therapeutic/dietary regimen or lifestyle recommendation that is suitable for the subject's predicted response to diet and/or exercise.

According to some embodiments, methods are provided for identifying a subject's metabolic genotype based on the identification in the subject's DNA one or more of the following allelic patterns, comprising any combination of the following IL-1 gene cluster haplotype markers: IL-1A +4845 (T), IL-1B +6054 (G), IL-1B +3877 (G), IL-1B +3954 (T), IL-1B −511 (C), IL-1B −3737 (C), IL-1B −511 (T), IL-1B −1468 (C), IL-1B +3954 (C), IL-1B −1468 (C), IL-1RN +2018 (T), IL-1RN 9005 (G), IL-1RN 315952 (C).

According to some embodiments, methods are provided for selecting an appropriate therapeutic/dietary regimen or lifestyle recommendation for a subject by identifying in a subject's DNA one or more of the following allelic patterns, comprising any combination of the following IL-1 gene cluster haplotype markers: IL-1A +4845 (T), IL-1B +6054 (G), IL-1B +3877 (G), IL-1B +3954 (T), IL-1B −511 (C), IL-1B −3737 (C), IL-1B −511 (T), IL-1B −1468 (C), IL-1B +3954 (C), IL-1B −1468 (C), IL-1RN +2018 (T), IL-1RN 9005 (G), IL-1RN 315952 (C), wherein the subject's genotype with respect to the loci provides information about the subject's likely response to calorie restricted diets, wherein the low calorie diet or liquid diet, and allows the selection of a therapeutic/dietary regimen or lifestyle recommendation that is suitable to the subject's susceptibility to adverse weight management issues.

According to some embodiments, methods are provided for selecting therapeutic/surgical/dietary or lifestyle in overweight or obese subjects carrying haplotype patterns CCT (3 SNPs) at rs4848306 (−3737)/rs1143623 (−1468)/rs16944 (−511)) or CCTC (4SNPs) at (rs4848306 (−3737)/rs1143623 (−1468)/rs16944 (−511)/rs1143634 (+3954)) loci, respectively, on IL1B gene and CG (2 SNPs) or TCG (3 SNPs) at rs315952/rs9005 or rs419598 (+2018)/rs315952/rs9005 loci, respectively, on IL1RN gene, wherein the presence of any one, any two or all four haplotypes will indicate that the subject is predisposed to resistance to weight loss when prescribed a calorie restricted diet or low calorie liquid diet.

According to some embodiments, methods are provided for selecting an appropriate therapeutic/dietary regimen or lifestyle recommendation for a subject by determining a subject's genotype with respect to any one, any two, any three, or any four, or all of the polymorphic loci selected from the following: IL1RN rs315952; IL1RN rs380092; IL1RN rs4251961; IL-1RN rs419598; IL-1RN 9005; IL1B +3877; IL1B +6054; IL1B rs4848306 (−3737); IL1B rs1143623 (−1468); IL-1B rs1143634 (+3954); IL1B 16944 (−511); IL1A rs17561; ADRB2 rs1042713; ADRB2 rs1042714; ADRB3 rs4994; MCR-4 rs2229616; MCR-4 rs12970134; MCR-4 rs477181; MCR-4 rs502933; MCR-4 4450508; PPARG rs1801282 and FABP2 rs1799883; wherein the presence of one or more alleles indicates that the subject has abnormal body fat content, wherein the subject has lower level of HDL, about 40 mg/dL or lower for men, and 50 mg/dL or lower for women, or higher level of LDL, about 100 mg/dL or above, or higher level of triglycerides, about 150 mg/dL or above, or any combination thereof.

According to some embodiments, lower level of HDL is 20-60 mg/dL or 50-59 mg/dL or 40-49 mg/dL or 30-39 mg/dL or <30 mg/dL; higher level of LDL is 100->190 mg/dL or 100-129 mg/dL or 130-159 mg/dL or 160-190 mg/dL or >190 mg/dL; and higher level of triglyceride is 150->500 mg/dL or 150-199 mg/dL or 200-500 mg/dL or >500 mg/dL.

According to some embodiments, methods are provided for selecting or screening subjects predisposed to abnormal blood lipid profiles based on the identification of alleles in the subject's DNA on one or more of the following genes: ADRB2 (rs1042713), IL1B (rs4848306 (−3737); rs1143623 (−1468); rs1143634 (+3954); and rs16944 (−511)), and MCR4 (rs12970134, rs2229616, rs477181 and rs502933) loci on the IL1A, IL1B, IL1RN, ADRB2 and ADRB3 genes, respectively. IL-1 pathway, ADRB2 and MCR4 genes affects fat metabolism and subjects with these alleles are resistant to weight loss will have higher body fat content.

According to some embodiments, methods are provided for selecting or screening subjects predisposed to lower levels of HDL, based on the identification in the subject's DNA one or more of the following alleles: ADRB2 (rs1042713; A/*), IL1B (rs1143623; −1468; G/G) and (rs16944; −511; C), and MCR4 (rs12970134; G) or (rs2229616; A) or (rs477181; G/*) or (rs502933; C/*) loci on the ADRB2, IL1B and MCR4 genes, respectively.

According to some embodiments, methods are provided for selecting or screening subjects predisposed to higher levels of triglycerides, based on the identification in the subject's DNA one or more of the following alleles: IL1B (rs1143623; −1468;C/C) or (rs1143634; +3954; C), and MCR4 (rs12970134; G/G) or (rs2229616; G/*) and IL1RN (rs9005; A) or (rs419598; +2018; C/C) in the IL1B, MCR4 and IL1RN genes respectively.

According to some embodiments, methods are provided for selecting or screening subjects predisposed to higher levels of LDL based on identification in the subject's DNA one or more of the following alleles: ADRB2 (rs1042713; A/A) and PPARG (rs1801282; G/*) in the ADRB2 and PPARG genes respectively.

According to some embodiments, methods are provided for selecting or screening subjects predisposed to loosing comparatively more body fat based on identification of the subject's haplotype patterns at MCR4 gene consisting of (rs12970134/rs477181/rs502933; GGC) and rs12970134/rs477181/rs502933/rs2229616 SNPs; GTAG) and ADRB2 gene (rs1042713/rs1042714; AC). IL-1 pathway, ADRB2 and MCR4 genes affects fat metabolism, and subjects predisposed to resistance to weight loss have higher body fat content.

According to some embodiments, methods are provided for selecting or screening subjects predisposed to lower levels of HDL based on identification of the subject's haplotype patterns at (rs12970134/rs477181/rs502933; GGC) and ADRB2 gene (rs1042713/rs1042714; AC).

According to some embodiments, methods are provided for selecting or screening subjects predisposed to higher levels of triglycerides based on identification of the subject's haplotype patterns at rs12970134/rs477181/rs502933/rs2229616 SNPs; GTAG) on the MCR4 gene.

According to some embodiments, kits are provided which include a means for determining a subject's genotype with respect to the IL1RN rs315952; IL1RN rs380092; IL1RN rs4251961; IL-1RN rs419598; IL-1RN 9005; IL1B rs1143633 (+3877); IL1B +6054; IL1B rs4848306 (−3737); IL1B rs1143623 (−1468); IL-1B rs1143634 (+3954); IL1B 16944 (−511); IL1A rs17561; ADRB2 rs1042713; ADRB2 rs1042714; ADRB3 rs4994; MCR-4 rs2229616; MCR-4 rs12970134; MCR-4 rs477181; MCR-4 rs502933; MCR-4 4450508; PPARG rs1801282 and FABP2 rs1799883. The kit may also contain a sample collection means. The kit may also contain a control sample either positive or negative or a standard and/or an algorithmic device for assessing the results and additional reagents and components.

According to some embodiments, kits are provided which include a means for detecting an allelic pattern according to the IL-1 gene cluster haplotype comprising any combination of the following markers: IL-1A +4845 (T), IL-1B +6054 (G), IL-1B +3877 (G), IL-1B +3954 (T), IL-1B −511 (C), IL-1B −3737 (C), IL-1B −511 (T), IL-1B −1468 (C), IL-1B +3954 (C), IL-1B −1468 (C), IL-1RN +2018 (T), IL-1RN 9005 (G), IL-1RN 315952 (C). The kit may also contain a sample collection means. The kit may also contain a control sample either positive or negative or a standard and/or an algorithmic device for assessing the results and additional reagents and components.

According to some embodiments, kits of the present invention may be in the form of a DNA test that will be used to provide diet and exercise recommendation based on a subject's genotype with respect to the IL1RN rs315952; IL1RN rs380092; IL1RN rs4251961; IL-1RN rs419598; IL-1RN 9005; IL1B rs1143633 (+3877); IL1B +6054; IL1B rs4848306 (−3737); IL1B rs1143623 (−1468); IL-1B rs1143634 (+3954); IL1B 16944 (−511); IL1A rs17561; ADRB2 rs1042713; ADRB2 rs1042714; ADRB3 rs4994; MCR-4 rs2229616; MCR-4 rs12970134; MCR-4 rs477181; MCR-4 rs502933; MCR-4 4450508; PPARG rs1801282 and FABP2 rs1799883. Information provided by a subject's genotype can help health professionals to develop personalized dietary and exercise interventions that will improve the prevention and treatment of obesity.

According to some embodiments, kits of the present invention may be in the form of a DNA test that will be used to provide diet and exercise recommendation based on a subject's genotype with respect to the IL-1 gene cluster haplotype comprising any combination of the following markers: IL-1A +4845 (T), IL-1B +6054 (G), IL-1B +3877 (G), IL-1B +3954 (T), IL-1B −511 (C), IL-1B −3737 (C), IL-1B −511 (T), IL-1B −1468 (C), IL-1B +3954 (C), IL-1B −1468 (C), IL-1RN +2018 (T), IL-1RN 9005 (G), IL-1RN 315952 (C). Information provided by a subject's genotype can help health professionals to develop personalized dietary and exercise interventions that will improve the prevention and treatment of obesity.

According to some embodiments, kits of the present invention may be in the form of a DNA test that will be used to provide information regarding a subject's HDL, LDL and triglyceride (TG) levels, with respect to the genotype in risk alleles from the group consisting of: IL1RN rs315952; IL1RN rs380092; IL1RN rs4251961; IL-1RN rs419598; IL-1RN 9005; IL1B rs1143633 (+3877); IL1B +6054; IL1B rs4848306 (−3737); IL1B rs1143623 (−1468); IL-1B rs1143634 (+3954); IL1B 16944 (−511); IL1A rs17561; ADRB2 rs1042713; ADRB2 rs1042714; ADRB3 rs4994; MCR-4 rs2229616; MCR-4 rs12970134; MCR-4 rs477181; MCR-4 rs502933; MCR-4 4450508; PPARG rs1801282 and FABP2 rs1799883.

According to some embodiments, a method of selecting patients for clinical trials for weight management therapies based on identification of a subject's genotype at IL1A (rs17561; +4845; T), IL1B SNPs, rs4848306 (−3737; C), rs1143623 (−1468; C), rs1143634 (+3954; C); and rs16944 (−511; T), IL1RN (rs315952; C), ADRB2 (rs1042713; G/*) and ADRB3 (rs4994; T) loci on the IL1A, IL1B, IL1RN, ADRB2 and ADRB3 genes, respectively, wherein the alleles predicts a subject's resistance to weight loss in response to low calorie diet or liquid diet.

According to some embodiments, a method of selecting patients for clinical trials for weight management therapies based on identification of a subject's haplotype patterns at (rs4848306 (−3737)/rs1143623 (−1468)/rs16944 (−511)/rs1143634 (+3954); CCTC) loci on IL1B gene and (rs315952/rs9005; CG) or (rs419598 (+2018)/rs315952/rs9005; TCG) loci on IL1RN gene, wherein the haplotype predicts a subject's resistance to weight loss in response to low calorie diet or liquid diet.

According to some embodiments, a method for selecting the responders of bariatric surgery based on identification of a subject's genotype at IL1A (rs17561; +4845; T), IL1B SNPs, rs4848306 (−3737; C), rs1143623 (−1468; C), rs1143633 (+3877; G); and rs16944 (−511; T), IL1RN (rs315952; C), ADRB2 (rs1042713; G/*) and ADRB3 (rs4994; T) loci, wherein carriers of one or more of the alleles predisposes the subject to resistance to weight loss in response to low calorie or liquid diet. Bariatric surgery, also known as weight loss surgery, refers to the various surgical procedures performed to treat obesity by modification of the gastrointestinal tract to reduce nutrient intake and/or absorption. The term does not include procedures for surgical removal of body fat such as liposuction or abdominoplasty. Subjects predicted to lose weight on the calorie restricted diets prior to bariatric surgery are more likely to maintain weight loss after the surgery (Still et. al, Arch Surg. 2007; 142(10):994-998).

According to some embodiments, a method for selecting the responders of bariatric surgery based on identification of a subject's haplotype patterns at (rs4848306 (−3737)/rs1143623 (−1468)/rs16944 (−511)/rs1143634 (+3954); CCTC) loci on IL1B gene and (rs315952/rs9005; CG) or (rs419598 (+2018)/rs315952/rs9005; TCG) loci on IL1RN gene. Subjects with these alleles are predicted to lose weight on the calorie restricted diets prior to bariatric surgery are more likely to maintain weight loss after the surgery (Still et. al, Arch Surg. 2007; 142(10):994-998).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other embodiments and advantages of the invention are set forth in the following detailed description and claims.

### BRIEF DESCRIPTION OF DRAWINGS

FIG. 1A shows the association of IL-1 cluster SNPs on percent change in body weight at three months in response to glycemic load.

FIG. 1B shows the association of IL-1 cluster SNPs on percent change in body weight at three months in response to glycemic load.

FIG. 2A shows the association of IL-1 cluster SNPs on change in body fat mass at three months in response to glycemic load.

FIG. 2B shows the association of IL-1 cluster SNPs on change in body fat mass at six months in response to glycemic load.

FIG. 3 shows the study design of the Geisinger Study.

FIG. 4 shows ADRB2 gene subject SNP rs1042713 and rs1042714 positions and the corresponding LD analysis.

FIG. 5 shows ADRB3 gene subject SNP rs4994 position.

FIG. 6 shows PPARG gene subject SNP rs180128 position.

FIG. 7 shows IL1A gene subject SNP rs17561 position.

FIG. 8 shows IL1B gene subject SNPs rs4848306, rs114362, and rs1143634 positions.

FIG. 9 shows IL1RN gene subject SNPs rs419598, rs31595, and rs9005 positions.

FIG. 10 shows the LD of SNPs in the IL-1 pathway. IL-1B SNPs (−3737, −1468 and −511) and IL1RN SNPs (rs315952 and rs9005) showed strong LD.

FIG. 11 shows MCR4 gene and 3′ flanking region. Also shown the position of subject SNPs, rs2229616, rs12970134, rs477181, and rs502933.

FIG. 12 shows MCR4 SNPs LD map. MCR4 rs12970134, rs477181 and rs502933 showed strong LD.

### DETAILED DESCRIPTION OF THE PREFERRED EMBODIMENTS

The invention bases upon the discovery of genotypes associated with resistance to weight-loss. Accordingly, the invention provides a genetic predisposition test that identifies a subject with elevated risk for lack of response to dietary regimen directed to weight-loss. The invention is also based upon the discovery of a genotype associated with increased risk for dyslipidemia. Dyslipidemia, as in this invention, is defined as the elevation of plasma cholesterol, triglycerides (TGs), or both, or a low high density lipoprotein (HDL) level that contributes to the development of atherosclerosis. Accordingly, the invention provides a genetic diagnostic test for identifying a subject who is predisposed to higher levels of LDL and triglycerides, or lower level of HDL. A subject with dyslipidemia has lower level of HDL, about 40 mg/dL or lower for men, and 50 mg/dL or lower for women, or higher level of LDL, about 100 mg/dL or above, or higher level of triglycerides, about 150 mg/dL or above, or all.

According to some embodiments, lower level of HDL is 20-60 mg/dL or 50-59 mg/dL or 40-49 mg/dL or 30-39 mg/dL or <30 mg/dL; higher level of LDL is 100->190 mg/dL or 100-129 mg/dL or 130-159 mg/dL or 160-190 mg/dL or >190 mg/dL; and higher level of triglyceride is 150->500 mg/dL or 150-199 mg/dL or 200-500 mg/dL or >500 mg/dL.

According to some embodiment of the invention, the presence, absence or predisposition to resistance to weight loss in a subject is determined by detecting in the subject a resistance to weight-loss-associated genotype. The presence of the genotype indicates that the subject has or is predisposed to resistance to weight loss.

According to some embodiments of the invention, a subject who lost about 3% or >3% of weight (total body weight measure) after about 4 months being enrolled in a low calorie diet (for example, about 1200-1500 kcal for women and 1500-1800 for men) were considered to have lost weight in response to low calorie diet in stage 1 and were classified as Group A (FIG. 3). In stage 2 (after the first 4 months, another about 4 months) all subjects who lost <3% weight in stage 1 were recommended a liquid diet of 1000 kcal (women) or 1200 kcal (men). Once on liquid diet, subjects who lost 5% or >5% of total body weight in an early stage were classified as Group B (early responders), and those who lost the same amount of weight, but at a later stage were categorized in Group C (late responders). Subjects, who did not respond to either stages (I or II), were classified as Group D (non-responders). (FIG. 3.)

According to some embodiments, the Group B early responders responded to liquid diet between 20-30 days, or 31-40 days, or 41-50 days, or 51-60 days, or 61-70 days, or 71-80 days, or 81-90 days, or 91-100 days, or 101-110 days, or 111-120 days. In some preferred embodiments, the Group B early responders responded between 20-120 days, or 20-60 days, or 30-60 days, or 30-120 days, or 60-120 days.

According to some embodiments, the Group C late responders responded to liquid diet between 120-130 days, or 131-140 days, or 141-150 days, or 151-160 days, or 161-170 days, or 171-180 days, or 181-190 days, or 191-200 days, or 201-210 days, or 211-220 days, or 221-230 days, or 231-240 days, or 241-250 days, or 251-260 days, or 261-270 days, or 271-280 days, or 281-290 days, or 291-300 days, or 301-310 days, or 311-320 days, or 321-330 days, or 331-340 days, or 341-350 days, or 351-360 days, or 361-370 days. In some preferred embodiments, the Group C late responders respond between 121-190 days, or 121-360 days, or 121-370 days, or 121-180 days, or 121-220 days, or 121-160 days, or 160-200 days, or 160-180 days, or 160-220 days, or 180-220 days, or 180-370 days.

According to some embodiments, methods are provided for screening subjects of the general population, such as teenagers or normal weight adults, who may be overly conscious of their weight, even if it falls into the so called “normal” range, which is BMI 18.5-24.9. According to this invention, an underweight subject has a BMI<18.5; an overweight subject in the range 25-29.9, an obese subject has a BMI of 30-39.9, and BMI of >40.0 is considered extremely obese. Identification of metabolic genotype in these subjects could provide health professionals with tools to discuss about the difficulties of a subject with a BMI of 25 to reach BMI of 22 with a lower-calorie diet alone.

According to some embodiments, methods and kits are provided for screening subjects for clinical trials for weight management, wherein an underweight subject has a BMI<18.5; an overweight subject in the range 25-29.9, an obese subject has a BMI of 30-39.9, and BMI of >40.0 is considered extremely obese. Identification of metabolic genotype in these subjects could provide health professionals with tools to discuss about the difficulties of a subject with a BMI of 25 to reach BMI of 22 with a lower-calorie diet alone.

According to some embodiment, absence of the genotype in tested loci indicates that the subject does not have or is not predisposed to resistance to weight loss. A symptom of resistance to weight loss is alleviated by detecting the presence of a resistance to weight loss associated genotype and guiding medical management of obese patients with recommendations for lifestyle changes which would include diet, exercise, therapeutics, or other medical interventions that are currently used to treat the major complications of obesity, particularly metabolic syndrome and fatty liver/non-alcoholic steatohepatitis (NASH).

The kits and methods of the present invention rely at least in part upon the finding that there is an association between the patterns of alleles of certain metabolic genes and the responsiveness of a subject to particular diet and exercise regime. That is, there is an association between the patterns of alleles of metabolic genes and weight management-related clinical outcomes and phenotypes. Certain genes impact various pathways that influence body weight, and have been associated with elevated risk for obesity and for their ability to differentiate subject's response to weight management interventions by genotype. For the purposes of this invention, such genes will be referred to as “metabolic genes” or “weight management genes”. These genes include, but are not limited to, the IL-1RN, IL-1A, IL-1B, ADRB2, ADRB3, PPARG and MCR4.

The present invention provides for Weight Management Tests to determine a subject's “metabolic genotype”, which involves determining a subject's genotype for one or more (e.g., 2, 3, 4, etc) metabolic genes. The results of such metabolic genotyping may be used to predict a subject's responsiveness to relative amounts of macronutrients and calorie restriction in the diet, with or without exercise or medical or surgical interventions, for weight loss. Identifying a subject's genotype may be used to pairing the subject with a therapeutic or nutrition or lifestyle alternation, or a combination thereof, thus devising a strategy to achieve and/or sustain weight loss. Thus, according to some embodiments, polymorphism genotyping results (for single polymorphisms or combinations) may be used to determine 1) genetic influence on weight management intervention/outcomes and 2) responsiveness to macronutrients and energy restriction in the diet, with or without exercise, for weight loss.

Collectively, determination of a subject's genotype for one or more metabolic genes allows interpretations that provide actionable information for selecting an appropriate therapeutic/dietary regimen or lifestyle recommendation for a subject. A subject's metabolic genotype is determined from a Weight Management Test designed to detect a subject's genetic polymorphism pattern with respect to one or more metabolic gene. By identifying relevant gene polymorphisms and genotype pattern results, the test can assess risk for likely weight management outcomes and provide the subject with guidance on the choice of nutrition and lifestyle interventions that match their personal genetic makeup.

A subject's single polymorphism metabolic genotype and/or composite metabolic genotype results may be classified according to their relationships to weight management risk, including what constitutes a “less responsive” or “more responsive” result from diet and/or exercise interventions, 2) their associated clinical or health-related biomarker outcomes, 3) their relationships to intervention choices for weight management, and 4) prevalence of each genotype.

Combinations of these gene variations affect 1) how subjects respond to specific macronutrients in their diet and 2) their different tendencies in energy metabolism that ultimately influence their ability to maintain or lose weight through exercise. A metabolic genotype determination will help healthy subjects identify a genetic risk for adverse weight management issues that have not yet manifested. Knowing gene-related risks early can assist in making personalized health decisions (nutrition, lifestyle) to preserve future health, as well as provide direction on how best to prioritize a subject's focus on nutrition and lifestyle choices to manage optimal body weight and body composition.

Information learned from a subject's metabolic genotype may be used to predict a subject's genetic risk for adverse weight management issues and response to particular diets (i.e., diets controlling for macronutrient proportions and calorie restrictions). The subject's genotype may be used to assess risk and allow for the selection of an appropriate therapeutic/dietary regimen or lifestyle recommendation. Generally, a subject's allelic pattern of one or more metabolic genes may be used to classify the subject's predicted responsiveness to macronutrients and energy restriction in the diet, with or without exercise, in a weight loss management program. Accordingly, a personalized weight management program may be selected for the subject based on subject's predicted response. For example, a weight management program may classify a subject's metabolic genotype into one of a series of nutrition categories and one of a series of exercise categories based upon that subject's predisposition for responsiveness to certain macronutrients and degree of exercise. The nutrition category, exercise category, or combination thereof may be selected for a subject based on subject's genetic patterns.

The method of the present invention can also be used in screening subjects of the general population, such as teenagers, who may be overly conscious of their weight, even if it falls into the so called “normal” range, which is BMI 18.5-24.9. According to this invention, an underweight subject has a BMI<18.5; an overweight subject in the range 25-29.9 and an obese subject has a BMI of 30 or greater. Identification of metabolic genotype in these subjects could provide health professionals with tools to discuss about the difficulties of a subject with a BMI of 25 to reach BMI of 22 with a lower-calorie diet alone.

According to some embodiments, methods are provided for predicting a subject's response to low calorie diet or liquid diet by determining a subject's genotype with respect to any one, any two, any three, or any four, or all of the polymorphic loci selected from the following: IL1RN, rs315952 locus; IL1RN, rs380092 locus; IL1RN, rs4251961 locus; IL1B rs1143633 (+3877) locus; IL1B +6054 locus; IL1B rs4848306 (−3737) locus; IL1B, rs1143623 (−1468) locus; IL1B, 16944 (−511) locus; IL1A, rs17561 locus; ADRB2, rs1042713 locus; ADRB3 rs4994 locus; MCR4 rs12970134 locus; MCR4 rs477181 locus; and MCR4 rs502933 locus, wherein the subject's genotype with respect to said loci provides information about the subject's likely response to low calorie diet or liquid diet, and allows the selection of a therapeutic/dietary regimen or lifestyle recommendation that is suitable to the subject's susceptibility to adverse weight management issues.

According to some embodiments, methods are provided for identifying a subject's genotype for pairing the subject with either therapeutic, or nutrition, or lifestyle alternation, or a combination thereof, thus devising a strategy to achieve and/or sustain weight loss.

Thus, according to some embodiments, methods are provided for identifying a subject's metabolic genotype by identifying the subject's genotype with respect to one or more (i.e., 2, 3, 4, or more) of IL1RN, rs315952 locus; IL1RN, rs380092 locus; IL1RN, rs4251961 locus; IL1B rs1143633 (+3877) locus; IL1B +6054 locus; IL1B rs4848306 (−3737) locus; IL1B, rs1143623 (−1468) locus; IL1B, 16944 (−511) locus; IL1A, rs17561 locus; ADRB2, rs1042713 locus; ADRB3 rs4994 locus; MCR4 rs12970134 locus; MCR4 rs477181 locus; and MCR4 rs502933 locus.

According to some embodiments, methods are provided for identifying a subject's metabolic genotype by identifying the subject's genotype with respect to the IL-1 gene cluster haplotype comprising the following markers: IL-1A +4845 (T), IL-1B +6054 (G), IL-1B +3877 (G), IL-1B +3954 (T), IL-1B −511 (C), IL-1B −3737 (C), IL-1B −511 (T), IL-1B −1468 (C), IL-1B +3954 (C), IL-1B −1468 (C), IL-1RN +2018 (T), IL-1RN 9005 (G), IL-1RN 315952 (C), wherein the haplotype predicts a subject's response to weight loss in response to low calorie diet or liquid diet.

According to some embodiments, methods are provided for selecting an appropriate therapeutic/dietary regimen or lifestyle recommendation for a subject by determining the subject's genotype with respect to any one, any two, any three, or any four, or all of the polymorphic loci or risk alleles selected from: IL-1RN rs315952; IL-1RN rs380092; IL-1RN rs4251961; IL-1B rs1143633 (+3877); IL-1B +6054; IL-1B rs4848306 (−3737); IL1B rs1143623 (−1468); IL-1B rs1143634 (+3954); IL1B rs16944 (−511); IL1A rs17561, wherein the subject's genotype with respect to said loci provides information about the subject's likely response to low calorie or liquid diet, and allows the selection of a therapeutic/dietary regimen or lifestyle recommendation that is suitable to the subject's susceptibility to adverse weight management issues, wherein the low calorie diet comprises a diet of 1200-1500 kcal for women, and 1500-1800 kcal for men, and liquid diet comprises a diet of 1000 kcal for women and 1200 kcal for men.

According to some embodiments of the invention carriers of the ADRB2 rs1042713 locus, IL-1A rs17581 locus, ADRB3 rs9449 locus, IL-1B rs4848306 (−3737) locus, IL-1B rs1143623 (−1468) locus, and IL-1B rs16944 (−511) and IL-1RN rs315952 locus genotypes were associated with resistance to weight loss under calorie restriction (FIG. 3). C allele of IL-1B rs4848306 (−3737) locus, T allele of IL-1B rs16944 (−511) and C allele at rs1143623 (−1468) loci were found associated with resistance to weight loss, when subjects restricted to moderate calorie restriction (Group A) were compared to subjects on liquid diet (1,000 to 1,200 kcal) (Group BC) in the Geisinger study (Table 7). C allele at IL-1RN SNP rs315952, G/* allele of ADRB2 SNP rs1042713, and T allele of IL-1A SNP rs17561 (+4845), were associated with resistance to weight loss in comparison groups calorie restriction (Group ABC) versus weight loss resistant group (Group D). T allele at ADRB3 (rs4994), G allele at IL-1B SNP rs1143623 and C allele at IL-1RN SNP rs315952 showed resistance to weight loss under calorie restriction (BC and D group comparison, FIG. 3). Subjects with allele C at ADRB3 SNP rs4994, allele C at IL-1B SNP rs1143623 and allele Tat IL-1RN SNP rs315952 showed response to weight loss under calorie restriction. In the low calorie diet responders versus resistant group comparison (A versus D group comparison in the Geisinger study of the present invention), ADRB2 SNP rs1042713 (G/*) and IL1A SNP rs17561 (+4845; T) alleles showed resistance to weight loss under calorie restriction (p=0.04). ADRB2 SNP rs1042713 (A/A) (p=0.048) and IL-1A SNP rs17561 (+4845; G) (p=0.039) alleles showed response to weight loss under calorie restriction. Calorie restriction refers to the categories shown in FIG. 3.

According to some embodiments, methods are provided for selecting an appropriate therapeutic/dietary regimen or lifestyle recommendation for a subject by identifying in a subject's DNA the IL-1 gene cluster haplotype comprising the following markers: IL-1A +4845 (T), IL-1B +6054 (G), IL-1B +3877 (G), IL-1B +3954 (T), IL-1B −511 (C), IL-1B −3737 (C), IL-1B −511 (T), IL-1B −1468 (C), IL-1B +3954 (C), IL-1B −1468 (C), IL-1RN +2018 (T), IL-1RN 9005 (G), IL-1RN 315952 (C), wherein the presence of the IL-1 gene cluster haplotype provides information about the subject's likely response to low calorie or liquid diet, and allows the selection of a therapeutic/dietary regimen or lifestyle recommendation that is suitable to the subject's susceptibility to adverse weight management issues.

According to some embodiments, the subject with the IL1RN, rs315952 (T) SNP predisposes a subject to respond to a low carbohydrate, calorie-restricted diet; regular exercise; or both. According to some embodiments, the subject with the IL1RN, rs315952 (T/T) genotype is predisposed to respond to a low carbohydrate, calorie-restricted diet; regular exercise; or both. According to some embodiments, the subject with the IL1RN, rs315952 (T*) genotype is predisposed to respond to a low carbohydrate, calorie-restricted diet; regular exercise; or both.

According to some embodiments, the subject with the IL1RN, rs380092 (A) SNP is predisposed to respond to a low carbohydrate, calorie-restricted diet; regular exercise; or both. According to some embodiments, the subject with the IL1RN, rs380092 (A/A) genotype is predisposed to respond to a low carbohydrate, calorie-restricted diet; regular exercise; or both. According to some embodiments, the subject with the IL1RN, rs380092 (A*) genotype is predisposed to respond to a low carbohydrate, calorie-restricted diet; regular exercise; or both.

According to some embodiments, the subject with the IL1RN, rs4251961 (C) SNP is predisposed to respond to a low carbohydrate, calorie-restricted diet; regular exercise; or both. According to some embodiments, the subject with the IL1RN, rs4251961 (C/C) genotype is predisposed to respond to a low carbohydrate, calorie-restricted diet; regular exercise; or both. According to some embodiments, the subject with the IL1RN, rs4251961 (C*) genotype is predisposed to respond to a low carbohydrate, calorie-restricted diet; regular exercise; or both.

According to some embodiments, the subject with the IL1B +3877 rs1143633 (G) SNP is predisposed to respond to a low carbohydrate, calorie-restricted diet; regular exercise; or both. According to some embodiments, the subject with the IL1B +3877 (G/G) genotype is predisposed to respond to a low carbohydrate, calorie-restricted diet; regular exercise; or both. According to some embodiments, the subject with the IL1B +3877 (G*) genotype is predisposed to respond to a low carbohydrate, calorie-restricted diet; regular exercise; or both.

According to some embodiments, the subject with the IL1B +6054 (G) SNP is predisposed to respond to a low carbohydrate, calorie-restricted diet; regular exercise; or both. According to some embodiments, the subject with the IL1B +6054 (G/G) genotype is predisposed to respond to a low carbohydrate, calorie-restricted diet; regular exercise; or both. According to some embodiments, the subject with the IL1B +6054 (G*) genotype is predisposed to respond to a low carbohydrate, calorie-restricted diet; regular exercise; or both.

According to some embodiments, the subject with the IL-1 gene cluster haplotype comprising any combination of the following alleles: IL-1A +4845 (T); IL-1A +4845 (G); IL-1B +6054 (G); IL-1B +3877 (G); IL-1B +3954 (T); IL-1B −511 (C); IL-1B −3737 (C); IL-1B −1468 (G); IL-1B −1468 (C), is predisposed to respond to a low carbohydrate, calorie-restricted diet; regular exercise; or both.

According to some embodiments, methods are provided selecting an appropriate therapeutic/dietary regimen or lifestyle recommendation for a subject comprises determining whether the subject has a genotype comprising any one or more alleles: (i) rs4848306 (−3737; C) of IL-1B; (ii) rs1143623 (−1468; C) of IL-1B; (iii) rs16944 (−511; T) of IL-1B; (iv) rs1042713 (G) of ADRB2; (v) rs17561 (+4845; T) of IL-1A; (vi) rs315952 (C) of IL-1RN; and (vii) rs4994 (T) of ADRB3; wherein the presence of the risk allele indicates that the subject is resistant to weight loss in response to a low calorie diet.

According to some embodiments, methods are provided selecting an appropriate therapeutic/dietary regimen or lifestyle recommendation for a subject comprises determining whether the subject has a genotype comprising any one or more risk alleles: (i) rs4848306 (−3737; T) of IL-1B; (ii) rs1143623 (−1468; G) of IL-1B; (iii) rs16944 (−511; C) of IL-1B; (iv) IL-1B +6054 (G); (v) IL-1B +3877 (G); (vi) rs1042713 (A/A) of ADRB2; (vii) rs17561 (+4845; G) of IL-1A; (viii) rs315952 (T) of IL-1RN; (ix) rs380092 of IL-1RN (A); (x) rs4251961 of IL-1RN (C); and (xi) rs4994 (C) of ADRB3; wherein the presence of the risk allele indicates that the subject is responsive to a low calorie diet to achieve weight loss.

According to some embodiments, methods are provided selecting an appropriate therapeutic/dietary regimen or lifestyle recommendation for a subject by detecting in the subject's DNA, any one or more of the following haplotype patterns: (i) rs315952 (C)/ rs9005 (G) of IL-1RN; (ii) rs419598(T)/rs315952 (C)/ rs9005 (G) of IL-1RN; (iii) rs16944 (T)/ rs1143623 (C)/ rs4848306 (C) of IL-1B; and (iv) rs1143634(C)/ rs16944 (T)/rs1143623(C)/rs4848306 (C) of IL-1B, wherein the presence of any one, any two, any three or all four haplotype patterns indicates that the subject is resistant to weight loss in response to a low calorie diet.

According to some embodiments, the therapeutic/dietary regimen comprises of administering a nutraceutical.

According to some embodiments, the methods above further comprise classifying the subject with respect to likely benefit from a therapeutic/dietary regimen or lifestyle change.

According to some embodiments, the low carbohydrate diet of the methods described above provide less than about 50 percent of total calories from carbohydrates.

According to some embodiments, the calorie-restricted diet of the methods described above restrict total calories to less than 95% of the subject's weight management level.

According to some embodiments, methods are provided for predicting a subject's response to calorie restricted diet or low calorie liquid diet, by identifying in the subject's DNA any one or more of the IL-1 gene cluster haplotype: IL-RN haplotype rs315952/rs9005 (315952, C)/(9005, G); IL-1RN haplotype rs419598/rs315952/rs9005 (+2018, T)/(315952, C)/(9005,G); IL-1B haplotype rs16944/rs1143623/rs4848306 (−511, T)/(−1468, C)/(−3737, C) or IL-1B haplotype rs1143634/rs16944/rs1143623/rs4848306 (+3954, C)/(−511, T)/(−1468, C)/(−3737, C); wherein the presence of any one, any two any three or all four haplotypes classifies the subject's genotype as predisposing the subject to resistance to weight loss when prescribed a low calorie diet or low calorie liquid diet.

According to some embodiments, methods are provided for selecting an appropriate therapeutic/dietary regimen or lifestyle recommendation for a subject by identifying in the subject's DNA any one or more of the IL-1 gene cluster haplotype: IL-RN haplotype rs315952/rs9005 (315952, C)/(9005, G); IL-1RN haplotype rs419598/rs315952/rs9005 (+2018, T)/(315952, C)/(9005,G); IL-1B haplotype rs16944/rs1143623/rs4848306 (−511, T)/(−1468, C)/(−3737, C) or IL-1B haplotype rs1143634/rs16944/rs1143623/rs4848306 (+3954, C)/(−511, T)/(−1468, C)/(−3737, C); wherein the presence of any one, any two any three or all four haplotypes classifies the subject's genotype as predisposing the subject to resistance to weight loss when prescribed a low calorie diet or low calorie liquid diet.

According to some embodiments, methods are provided for selecting an appropriate therapeutic/dietary regimen or lifestyle recommendation for a subject by detecting an allelic pattern according to the IL-1 gene cluster haplotype comprising the following markers: IL-RN haplotype rs315952/rs9005 (315952, C)/(9005, G); IL-1RN haplotype rs419598/rs315952/rs9005 (+2018, T)/(315952, C)/(9005,G); IL-1B haplotype rs16944/rs1143623/rs4848306 (−511, T)/(−1468, C)/(−3737, C) or IL-1B haplotype rs1143634/rs16944/rs1143623/rs4848306 (+3954, C)/(−511, T)/(−1468, C)/(−3737, C); wherein the presence of allelic pattern is predictive of the subject's response to diet and/or exercise; and by selecting a therapeutic/dietary regimen or lifestyle recommendation that is suitable for the subject's predicted response to a low calorie diet or low calorie liquid diet, regular exercise or all.

According to some embodiments, methods are provided identifying a subject's metabolic genotype comprising: detecting an allelic pattern according to the IL-1 gene cluster haplotype comprising the following markers: IL-RN haplotype rs315952/rs9005 (315952, C)/(9005, G); IL-1RN haplotype rs419598/rs315952/rs9005 (+2018, T)/(315952, C)/(9005,G); IL-1B haplotype rs16944/rs1143623/rs4848306 (−511, T)/(−1468, C)/(−3737, C) or IL-1B haplotype rs1143634/rs16944/rs1143623/rs4848306 (+3954, C)/(−511, T)/(−1468, C)/(−3737, C).

According to some embodiments, methods are provided for determining polymorphisms in genes in a subject for their association with lower level of HDL, by determining the subject's genotype with respect to any one, any two, any three, or any four, or all of the polymorphic loci or risk alleles selected from the group consisting of: ADRB2 (rs1042713; A/*); IL-1B (rs1143623; −1468; G/G); IL-1B (rs16944; −511; C); MCR4 (rs12970134; G); MCR4 (rs2229616; A); MCR4 (rs477181; G/*) and MCR4 (rs502933; C/*).

According to some embodiments, methods are provided for selecting an appropriate therapeutic/dietary regimen or lifestyle recommendation for a subject by determining the subject's genotype with respect to any one, any two, any three, or any four, or all of the polymorphic loci or risk alleles selected from the group consisting of: ADRB2 (rs1042713; A/*); IL-1B (rs1143623; −1468; G/G); IL-1B (rs16944; −511; C); MCR4 (rs12970134; G); MCR4 (rs2229616; A); MCR4 (rs477181; G/*) and MCR4 (rs502933; C/*); wherein presence of any one, any two, any three or any four loci indicates that said subject is predisposed to lower levels of HDL, wherein the subject has lower level of HDL is about <60 mg/dL (for e.g. 20-60 mg/dL or 50-59 mg/dL or 40-49 mg/dL or 30-39 mg/dL or <30 mg/dL).

According to some embodiments, methods are provided for determining polymorphisms in genes in a subject for their association with higher levels of triglycerides, by determining the subject's genotype with respect to any one, any two, any three, or any four, or all of the polymorphic loci or risk alleles selected from the group consisting of: IL-1B (rs1143623; −1468; C/C); IL-1B (rs1143634; +3954; C); MCR4 (rs12970134; G/G); MCR4 (rs2229616; G/*); IL-1RN (rs9005; A); IL-1RN (rs419598; +2018; C/C).

According to some embodiments, methods are provided for selecting an appropriate therapeutic/dietary regimen or lifestyle recommendation for a subject by determining the subject's genotype with respect to any one, any two, any three, or any four, or all of the polymorphic loci or risk alleles selected from: IL-1B (rs1143623; −1468; C/C); IL-1B (rs1143634; +3954; C); MCR4 (rs12970134; G/G); MCR4 (rs2229616; G/*); IL-1RN (rs9005; A); IL-1RN (rs419598; +2018; C/C); wherein presence of any one, any two, any three or any four loci indicates that the subject is predisposed to higher levels of triglycerides, wherein the subject has about >150 mg/dL triglyceride (for e.g. about 150->500 mg/dL or 150-199 mg/dL or 200-500 mg/dL or >500 mg/dL).

According to some embodiments, methods are provided for determining polymorphisms in genes in a subject for their association with higher levels of LDL, by determining the subject's genotype with respect to any one, or any two of the polymorphic loci selected from the ADRB2 (rs1042713: A/A) locus and PPARG (rs1801282; G/*) locus, wherein presence one or two alleles indicates that the subject is predisposed to higher levels of LDL, wherein the subject has LDL about >100 mg/dL (for e.g. about 100->190 mg/dL or 100-129 mg/dL or 130-159 mg/dL or 160-190 mg/dL or >190 mg/dL).

According to some embodiments, methods are provided for selecting an appropriate therapeutic/dietary regimen or lifestyle recommendation for a subject by determining the subject's genotype with respect to any one or any two of the polymorphic loci selected from the ADRB2 (rs1042713: A/A) locus and PPARG (rs1801282; G/*) locus, wherein presence one or two alleles indicates that the subject is predisposed to higher levels of LDL.

According to some embodiments, methods are provided for selecting an appropriate therapeutic/dietary regimen or lifestyle recommendation for a subject by determining the subject's genotype with respect to a haplotype pattern from: MCR4 gene haplotype, comprising rs12970134 (G)/rs477181(G)/rs502933(C) (GGC) and rs12970134 (G)/rs477181 (T)/rs502933 (A)/rs2229616 (G); and haplotype pattern at ADRB2 gene, rs1042713 (A)/rs1042714 (C), wherein the presence of one or more haplotypes indicates that the subject has abnormal body fat content, wherein the subject is predisposed to lower level of HDL or higher level of triglycerides.

According to some embodiments, methods are provided for selecting an appropriate therapeutic/dietary regimen or lifestyle recommendation for a subject by determining the subject's genotype with respect to a haplotype pattern at MCR4 gene, rs12970134 (G)/rs477181(G)/rs502933(C) (GGC) or haplotype pattern at ADRB2 gene, rs1042713 (A)/rs1042714 (C), wherein the presence of one or more haplotypes indicates that the subject is predisposed to abnormal body fat content, wherein the subject is predisposed to lower level of HDL.

According to some embodiments, methods are provided for selecting an appropriate therapeutic/dietary regimen or lifestyle recommendation for a subject by determining the subject's genotype with respect to a haplotype pattern at MCR4 gene, rs12970134 (G)/rs477181 (T)/rs502933 (A)/rs2229616 (G); wherein the presence of the haplotype indicates that the subject is predisposed to abnormal body fat content, wherein the subject is predisposed to higher level of triglycerides.

According to some embodiments, methods are provided for selecting an appropriate therapeutic/dietary regimen or lifestyle recommendation for a subject by determining the subject's genotype with respect to a haplotype pattern at ADRB2 gene, rs1042713 (A)/rs1042714 (C); wherein the presence of the haplotype indicates that the subject is predisposed to abnormal body fat content, wherein the subject is predisposed to higher level of triglycerides.

According to some embodiments, a kit is provided for determining if a subject is resistant to achieve weight loss, by detecting in said subject's DNA, the one or more alleles, selected from: (i) rs315952 of IL1RN; (ii) rs380092 of IL1RN; (iii) rs4251961 of IL1RN; (iv) rs16944 of IL1B (−511); (v) rs4848306 of IL1B (−3737); (vi) rs1143623 of IL1B (−1468); (vii) rs1143634 of IL-1B (+3954); (viii) rs17561 of IL-1A (+4845); (ix) rs1042713 of ADRB2; (x) rs4994 of ADRB3; (xi) rs12970134 of MCR4; (xii) rs477181; (xiii) rs502933 of MCR4F; and (xiv) rs1801282 of PPARG, wherein the presence of one or more allele indicates the subject's response to a low calorie diet. The kit may also contain a control sample either positive or negative or a standard and/or an algorithmic device for assessing the results and additional reagents and components. Information provided by said subject's genotype can help health professionals to develop personalized dietary and exercise interventions that will improve the prevention and treatment of obesity.

According to some embodiments, a kit is provided for determining if a subject is responsive to low calorie diet by determining whether the subject has a genotype comprising the alleles selected from: (i) rs4848306 (−3737; C) of IL-1B; (ii) rs1143623 (−1468; C) of IL-1B; (iii) rs16944 (−511; T) of IL-1B; (iv) rs1042713 (G) of ADRB2; (v) rs17561 (+4845; T) of IL-1A; (vi) rs315952 (C) of IL-1RN; and (vii) rs4994 (T) of ADRB3; wherein the presence of any one or more of the alleles indicate that the subject is resistant to weight loss in response to a low calorie diet, or liquid diet, or both.

According to some embodiments, a kit is provided for determining if a subject is responsive to low calorie diet by determining whether the subject has a genotype comprising said risk alleles selected from: (i) rs4848306 (−3737; T) of IL-1B; (ii) rs1143623 (−1468; G) of IL-1B; (iii) rs16944 (−511; C) of IL-1B; (iv) rs1042713 (A/A) of ADRB2; (v) rs17561 (+4845; G) of IL-1A; (vi) rs315952 (T) of IL-1RN; and (vii) rs4994 (C) of ADRB3; wherein the presence of any one or more of the alleles indicate that the subject is responsive to a low calorie diet, or liquid diet, or both, to achieve weight loss.

According to some embodiments, a kit is provided for determining if a subject is responsive to low calorie diet by detecting in the subject's DNA, the following haplotype patterns, selected from: (i) rs315952 (C)/rs9005 (G) of IL-1RN; (ii) rs419598(T)/rs315952(C)/rs9005(G) of IL-1RN; (iii) rs16944 (T)/rs1143623 (C)/rs4848306 (C) of IL-1B; and (iv) rs1143634 (C)/rs16944 (T)/rs1143623(C)/rs4848306 (C) of IL-1B, wherein the presence of any one, any two, any three or all four haplotype patterns indicate that the subject is resistant to weight loss in response to a low calorie diet, or liquid diet, or both.

According to some embodiments, a kit is provided for determining if a subject is resistant to achieve weight loss by determining the subject's genotype with respect to any one, any two, any three, or any four, or all of the alleles selected from: IL1RN, rs315952; IL1RN, rs380092; IL1RN, rs4251961; IL1B rs1143633 (+3877); IL1B +6054; IL1B rs4848306 (−3737); IL1B, rs1143623 (−1468); IL-1B rs1143634 (+3954; C); IL1B, 16944 (−511); IL1A, rs17561; ADRB2, rs1042713; ADRB3 rs4994; MCR4 rs12970134; MCR4 rs477181; and MCR4 rs502933; wherein the presence of one or more alleles indicates that the subject is predisposed to lower level of HDL, or higher level of triglycerides, or both.

According to some embodiments, a kit is provided for determining a subject's genotype with respect to any one, any two, any three, or any four or all of the alleles selected from: ADRB2 (rs1042713; A/*); IL-1B (rs1143623; −1468; G/G); IL-1B (rs16944; −511; C); MCR4 (rs12970134; G); MCR4 (rs2229616; A); MCR4 (rs477181; G/*) and MCR4 (rs502933; C/*), wherein the presence of one or more alleles indicates that the subject is predisposed to lower levels of HDL.

According to some embodiments, a kit is provided for determining a subject's genotype with respect to any one, any two, any three, or any four or all of the alleles selected from: IL-1B (rs1143623; −1468 C/C); IL-1B (rs1143634; +3954; C); MCR4 (rs1290134; G/G); MCR4 (rs2229616; G/*); IL-1RN (rs9005; A); IL-1RN (rs419598; +2018; C/C), wherein the presence of one or more alleles indicates that the subject is predisposed to higher levels of triglycerides.

According to some embodiments, a kit is provided for determining if a subject is responsive low calorie diet by determining the subject's genotype with respect to any one, or any two of the risk alleles selected from: ADRB2 (rs1042713; A/A) locus and PPARG (rs1801282; G/*) locus, wherein presence of one or two alleles indicate that the subject is predisposed to higher levels of LDL.

According to some embodiments, a kit is provided for determining if a subject is responsive low calorie diet by determining the subject's genotype with respect to a haplotype pattern: MCR4 gene GGC haplotype, rs12970134(G)/rs477181(G)/rs502933 (C), and ADRB2 gene haplotype AC, rs1042713(G)/rs1042714(T), wherein the presence of one or more haplotypes indicates that the subject is predisposed to lower level of HDL.

According to some embodiments, a kit is provided for determining if a subject is responsive low calorie diet by determining the subject's genotype with respect to a haplotype pattern from: at MCR4 gene, rs12970134 (G)/rs477181 (T)/rs502933 (A)/rs2229616 (G) and at ADRB2 gene, rs1042713 (A)/rs1042714 (C); wherein the presence of any one or both haplotype patterns indicate that the subject is predisposed to higher level of triglycerides.

According to some embodiments, methods are provided for selecting an appropriate therapeutic/dietary regimen or lifestyle recommendation for a subject comprising genotyping the subject at one or more loci: IL-1B, IL-1A, IL-1RN, ADRB2, ADRB3, and MCR4, wherein the presence of one or more risk allele within the locus is predictive of the subject's predisposition to weight loss in response to low calorie diet, or liquid diet, or both.

According to some embodiments, methods are provided for selecting an appropriate therapeutic/dietary regimen or lifestyle recommendation for a subject comprises genotyping the subject at the SNP rs4848306 of IL-1B marker −3737, wherein the presence of allele C indicates that the subject is resistant, and presence of allele T indicates that the subject is responsive to weight loss in response to a low calorie diet, or liquid diet, or both.

According to some embodiments, methods are provided for selecting an appropriate therapeutic/dietary regimen or lifestyle recommendation for a subject comprises genotyping the subject at the SNP rs1143623 of marker −1468 of IL-1B, wherein the presence of allele C indicates that the subject is resistant, and presence of allele G indicates that the subject is responsive to weight loss in response to a low calorie diet, or liquid diet, or both. In further embodiment, methods are provided wherein presence of homozygous G/G allele is predictive that the subject is predisposed to lower level of HDL in response to low calorie diet, or liquid diet, or both. In further embodiments, methods are provided wherein presence of homozygous C/C allele is predictive that the subject is predisposed to higher level of triglyceride in response to low calorie diet, or liquid diet, or both.

According to some embodiments, methods are provided for selecting an appropriate therapeutic/dietary regimen or lifestyle recommendation for a subject comprises genotyping said subject at the SNP rs16944 of IL-1B marker −511, wherein the presence of allele T indicates that the subject is resistant, and presence of allele C indicates that the subject is responsive to weight loss in response to a low calorie diet, or liquid diet, or both. In further embodiments, methods are provided wherein presence of heterozygous allele C is predictive that the subject is predisposed to lower level of HDL in response to low calorie diet, or liquid diet, or both.

According to some embodiments, methods are provided for selecting an appropriate therapeutic/dietary regimen or lifestyle recommendation for a subject comprises genotyping the subject at the SNP rs1042713 of ADRB2, wherein the presence of heterozygous allele G indicates that said subject is resistant, and presence of homozygous allele A indicates that the subject is responsive to weight loss in response to a low calorie diet, or liquid diet, or both. In further embodiment, presence of heterozygous allele G, is predictive that the subject is predisposed to lower level of HDL in response to low calorie diet, or liquid diet, or both. In further embodiment, presence of homozygous allele A is predictive that the subject is predisposed to higher level of LDL in response to low calorie diet, or liquid diet, or both.

According to some embodiments, methods are provided for selecting an appropriate therapeutic/dietary regimen or lifestyle recommendation for a subject comprises genotyping the subject at the SNP rs17561 of marker +4845 of IL-1A, wherein the presence of allele T indicates that the subject is resistant, and presence of allele G indicates that the subject is responsive to weight loss in response to a low calorie diet, or liquid diet, or both.

According to some embodiments, methods are provided for selecting an appropriate therapeutic/dietary regimen or lifestyle recommendation for a subject comprises genotyping the subject at the SNP rs315952 of IL-1RN, wherein the presence of allele C indicates that the subject is resistant, and presence of allele T indicates that the subject is responsive to weight loss in response to a low calorie diet, or liquid diet, or both.

According to some embodiments, methods are provided for selecting an appropriate therapeutic/dietary regimen or lifestyle recommendation for a subject comprises genotyping the subject at the SNP rs4994 of ADRB3, wherein the presence of allele T indicates that the subject is resistant, and presence of allele C indicates that the subject is responsive to weight loss in response to a low calorie diet, or liquid diet, or both.

According to some embodiments, methods are provided for selecting an appropriate therapeutic/dietary regimen or lifestyle recommendation for a subject comprises detecting in the subject allele G at +6054 marker of IL-1B, wherein the presence of the allele indicates that the subject is responsive to weight loss in response to a low calorie diet, wherein low calorie diet is a low glycemic diet under calorie restriction.

According to some embodiments, methods are provided for selecting an appropriate therapeutic/dietary regimen or lifestyle recommendation for a subject comprises detecting in the subject allele G at +3877 marker of IL-1B, wherein the presence of the allele indicates that the subject is responsive to weight loss in response to a low calorie diet, wherein low calorie diet is a low glycemic diet under calorie restriction.

According to some embodiments, methods are provided for selecting an appropriate therapeutic/dietary regimen or lifestyle recommendation for a subject comprises detecting in the subject allele A at SNP rs380092 of IL-1RN, wherein the presence of the allele indicates that the subject is responsive to weight loss in response to a low calorie diet, wherein low calorie diet is a low glycemic diet under calorie restriction.

According to some embodiments, methods are provided for selecting an appropriate therapeutic/dietary regimen or lifestyle recommendation for a subject comprises detecting in the subject allele C at SNP rs4251961 of IL-1RN, wherein the presence of the allele indicates that the subject is responsive to weight loss in response to a low calorie diet, wherein low calorie diet is a low glycemic diet under calorie restriction.

According to some embodiments, methods are provided for selecting an appropriate therapeutic/dietary regimen or lifestyle recommendations for a subjects comprising genotyping the subject for composite genotype at one or more loci selected from: IL-1B, IL-1A, IL-1RN, ADRB2, ADRB3, and MCR4, wherein the presence of one or more the composite genotypes within any one of the loci is predictive of the subject's predisposition to weight loss in response to low calorie diet, or liquid diet, or both.

According to some embodiments, methods are provided for selecting an appropriate therapeutic/dietary regimen or lifestyle recommendation for a subject comprises the steps of: a) genotyping the subject at: (i) SNP rs315952 of IL-1RN; and (ii) SNP rs9005 of IL-1RN; b) determining whether the subject has a composite genotype comprising the allelic pattern or haplotype of: heterozygous allele C at SNP rs315952 of IL-1RN and heterozygous allele G at rs9005 of IL-1RN; wherein the presence of the haplotype indicates that the subject is resistant to weight loss in response to a low calorie or liquid diet.

According to some embodiments, methods are provided for selecting an appropriate therapeutic/dietary regimen or lifestyle recommendation for a subject comprises the steps of: a) genotyping the subject at: (i) SNP rs419598 of IL-1RN; (ii) SNP rs315952 of IL-1RN; and (iii) SNP rs9005 of IL-1RN; b) determining whether the subject has a composite genotype comprising the allelic pattern or haplotype of: heterozygous allele T at SNP rs419598 of IL-1RN, heterozygous allele C at SNP rs315952 of IL-1RN, and heterozygous allele G at rs9005 of IL-1RN; wherein the presence of the haplotype indicates that the subject is resistant to weight loss in response to a low calorie or liquid diet.

According to some embodiments, methods are provided for selecting an appropriate therapeutic/dietary regimen or lifestyle recommendation for a subject comprises the steps of: a) genotyping the subject at: (i) SNP rs16944 of IL-1B; (ii) SNP rs1143623 of IL-1B; and (iii) SNP rs4848306 of IL-1B; b) determining whether the subject has a composite genotype comprising the allelic pattern or haplotype of: heterozygous allele T at SNP rs16944 of IL-1B, heterozygous allele C at SNP rs1143623 of IL-1B, and heterozygous allele C at SNP rs4848306 of IL-1B; wherein the presence of the haplotype indicates that the subject is resistant to weight loss in response to a low calorie or liquid diet.

According to some embodiments, methods are provided for selecting an appropriate therapeutic/dietary regimen or lifestyle recommendation for a subject comprises the steps of: a) genotyping the subject at: (i) SNP rs1143634 of IL-1B; (ii) SNP rs16944 of IL-1B; (iii) SNP rs1143623 of IL-1B; and (iv) SNP rs4848306 of IL-1B; b) determining whether the subject has a composite genotype comprising the allelic pattern or haplotype of: heterozygous allele C at SNP rs1143634 of IL-1B, heterozygous allele T at SNP rs16944 of IL-1B, heterozygous allele C at SNP rs1143623 of IL-1B, and heterozygous allele C at SNP rs4848306 of IL-1B; wherein the presence of the haplotype indicates that the subject is resistant to weight loss in response to a low calorie or liquid diet.

According to some embodiments, methods are provided for selecting an appropriate therapeutic/dietary regimen or lifestyle recommendation for a subject comprises the steps of: a) genotyping the subject at: (i) SNP rs1042713 of ADRB2; and (ii) SNP rs1042714 of ADRB2; b) determining whether the subject has a composite genotype comprising the allelic pattern or haplotype of: heterozygous allele A at SNP rs1042713 of ADRB2, and heterozygous allele C at SNP rs1042714 of ADRB2; wherein the presence of the haplotype is predictive that the subject is predisposed to lower level of HDL and higher level of triglyceride in response to a low calorie or liquid diet.

According to some embodiments, methods are provided for selecting an appropriate therapeutic/dietary regimen or lifestyle recommendation for a subject comprises the steps of: a) genotyping the subject at: (i) SNP rs12970134 of MCR4; (ii) SNP rs477181 of MCR4; and (iii) SNP rs502933 of MCR4; b) determining whether the subject has a composite genotype comprising the allelic pattern or haplotype of: heterozygous allele G at SNP rs12970134 of MCR4, heterozygous allele G at SNP rs477181 of MCR4, and heterozygous allele C at SNP rs502933 of MCR4; wherein the presence of the haplotype is predictive that the subject is predisposed to lower level of HDL in response to a low calorie or liquid diet.

According to some embodiments, methods are provided for selecting an appropriate therapeutic/dietary regimen or lifestyle recommendation for a subject comprises the steps of: a) genotyping the subject at: (i) SNP rs12970134 of MCR4; (ii) SNP rs477181 of MCR4; (iii) SNP rs502933 of MCR4; and (iv) SNP rs2229616 of MCR4; b) determining whether the subject has a composite genotype comprising the allelic pattern or haplotype of: heterozygous allele G at SNP rs12970134 of MCR4, heterozygous allele T at SNP rs477181 of MCR4, heterozygous allele A at SNP rs502933 of MCR4, and heterozygous allele G at rs2229616 of MCR4; wherein the presence of the haplotype is predictive that the subject is predisposed to higher level of triglyceride in response to a low calorie or liquid diet.

According to some embodiments, a kit is provided for determining a subject's response to low calorie or liquid diet toward achieving weight loss comprising reagents and instructions for genotyping the subject at one or more loci selected from: IL-1B, IL-1A, IL-1RN, ADRB2, ADRB3, and MCR4, wherein the presence of one or more risk allele within the locus is predictive of the subject's predisposition to weight loss in response to low calorie diet, or liquid diet, or both.

According to some embodiments, a kit is provided for determining a subject's response to low calorie or liquid diet toward achieving weight loss comprises reagents and instructions for detecting in the subject an allele at SNP rs4848306 of IL-1B marker −3737, wherein the reagents comprises primers, buffers, salts for detecting said allele.

According to some embodiments, a kit is provided for determining a subject's response to low calorie or liquid diet toward achieving weight loss comprises reagents and instructions for detecting in the subject an allele at SNP rs1143623 of IL-1B marker −1468, wherein the reagents comprises primers, buffers, salts for detecting said allele.

According to some embodiments, a kit is provided for determining a subject's response to low calorie or liquid diet toward achieving weight loss comprises reagents and instructions for detecting in the subject an allele at SNP rs16944 of IL-1B marker −511, wherein the reagents comprises primers, buffers, salts for detecting the allele.

According to some embodiments, a kit is provided for determining a subject's response to low calorie or liquid diet toward achieving weight loss comprises reagents and instructions for detecting in the subject an allele at SNP rs1042713 of ADRB2, wherein the reagents comprises primers, buffers, salts for detecting the allele.

According to some embodiments, a kit is provided for determining a subject's response to low calorie or liquid diet toward achieving weight loss comprises reagents and instructions for detecting in the subject an allele at SNP rs17561 of IL-1A marker +4845, wherein the reagents comprises primers, buffers, salts for detecting the allele.

According to some embodiments, a kit is provided for determining a subject's response to low calorie or liquid diet toward achieving weight loss comprises reagents and instructions for detecting in the subject an allele at SNP rs315952 of IL-1RN, wherein the reagents comprises primers, buffers, salts for detecting the allele.

According to some embodiments, a kit is provided for determining a subject's response to low calorie or liquid diet toward achieving weight loss comprises reagents and instructions for detecting in the subject an allele at SNP rs4994 of ADRB3, wherein the reagents comprises primers, buffers, salts for detecting the allele.

According to some embodiments, a kit is provided for determining said subject's response to low calorie or liquid diet toward achieving weight loss comprises reagents and instructions for detecting in the subject an allele G at +6054 marker of IL-1B, wherein the reagents comprises primers, buffers, salts for detecting the allele.

According to some embodiments, a kit is provided for determining a subject's response to low calorie or liquid diet toward achieving weight loss comprises reagents and instructions for detecting in the subject an allele G at +3877 marker of IL-1B, wherein the reagents comprises primers, buffers, salts for detecting the allele.

According to some embodiments, a kit is provided for determining a subject's response to low calorie or liquid diet toward achieving weight loss comprises reagents and instructions for detecting in the subject an allele A at SNP rs380092 of IL-1RN, wherein the reagents comprises primers, buffers, salts for detecting the allele.

According to some embodiments, a kit is provided for determining a subject's response to low calorie or liquid diet toward achieving weight loss comprises reagents and instructions for detecting in the subject an allele at C at SNP rs4251961 of IL-1RN, wherein the reagents comprises primers, buffers, salts for detecting the allele.

According to some embodiments, a kit is provided for determining a subject's response to low calorie or liquid diet toward achieving weight loss comprising genotyping the subject for composite genotype at one or more loci selected from: IL-1B, IL-1A, IL-1RN, ADRB2, ADRB3, and MCR4, wherein the presence of one or more risk allele within the locus is predictive of the subject's predisposition to weight loss in response to low calorie diet, or liquid diet, or both.

According to some embodiments, a kit is provided for determining a subject's composite genotype, comprises reagents and instructions for genotyping said subject at: (i) SNP rs315952 of IL-1RN; and (ii) SNP rs9005 of IL-1RN; wherein the reagents comprises primers, buffers, salts for detecting the allele.

According to some embodiments, a kit is provided for determining a subject's composite genotype, comprising reagents and instructions for genotyping the subject at: (i) SNP rs419598 of IL-1RN; (ii) SNP rs315952 of IL-1RN; and (iii) SNP rs9005 of IL-1RN; wherein the reagents comprises primers, buffers, salts for detecting the allele.

According to some embodiments, a kit is provided for determining a subject's composite genotype, comprising reagents and instructions for genotyping the subject at: (i) SNP rs16944 of IL-1B; (ii) SNP rs1143623 of IL-1B; and (iii) SNP rs4848306 of IL-1B; wherein the reagents comprises primers, buffers, salts for detecting the allele.

According to some embodiments, a kit is provided for determining a subject's composite genotype, comprising reagents and instructions for genotyping the subject at: (i) SNP rs1143634 of IL-1B; (ii) SNP rs16944 of IL-1B; (iii) SNP rs1143623 of IL-1B; and (iv) SNP rs4848306 of IL-1B; wherein the reagents comprises primers, buffers, salts for detecting the allele.

According to some embodiments, a kit is provided for determining a subject's composite genotype, comprising reagents and instructions for genotyping the subject at: (i) SNP rs1042713 of ADRB2; and (ii) SNP rs1042714 of ADRB2; b) determining whether the subject has a composite genotype comprising the allelic pattern or haplotype of: heterozygous allele A at SNP rs1042713 of ADRB2, and heterozygous allele C at SNP rs1042714 of ADRB2; wherein the presence of said haplotype is predictive that the subject is predisposed to lower level of HDL and higher level of triglyceride.

According to some embodiments, a kit is provided for determining a subject's composite genotype, comprising reagents and instructions for: a) genotyping the subject's DNA for one or more of the following alleles: (i) SNP rs12970134 of MCR4; (ii) SNP rs477181 of MCR4; and (iii) SNP rs502933 of MCR4; b) determining whether the subject has a composite genotype comprising the allelic pattern or haplotype of: heterozygous allele G at SNP rs12970134 of MCR4, heterozygous allele G at SNP rs477181 of MCR4, and heterozygous allele C at SNP rs502933 of MCR4; wherein the presence of the haplotype is predictive that the subject is predisposed to lower level of HDL.

According to some embodiments, a kit is provided for determining a subject's composite genotype, comprising reagents and instructions for: a) genotyping the subject's DNA for one or more of the following alleles: (i) SNP rs12970134 of MCR4; (ii) SNP rs477181 of MCR4; (iii) SNP rs502933 of MCR4; and (iv) SNP rs2229616 of MCR4; wherein the reagents comprises primers, buffers, salts for detecting the allele.

Nutrition Categories

The Geisinger Study was performed in two main stages. The stages were identified based on the numbers of calorie consumed. In stage 1 (subjects were subjected to this “low calorie diet” for about 4 months), enrolled women were recommended a diet of 1100-1800 kcal. In some embodiments, women were provided a diet of 1700-1800 kcal, or 1600-1700 kcal, or 1500-1600 kcal or 1400-1500 kcal or 1300-1400 kcal or 1200-1300 kcal or 1100-1200 kcal. In some embodiments, women were provided a diet of 1200-1500 kcal, or 1100-1500 kcal, or 1500-1800 kcal. In a preferred embodiment, women were provided a diet of 1200 kcal.

In stage 1, men were provided with a low calorie diet in the range of 1400-2200 kcal. In some embodiments, men were provided a diet of 2100-2200 kcal, or 2000-2100 kcal, or 1900-2000 kcal, or 1800-1900 kcal, or 1700-1800 kcal, or 1600-1700 kcal, or 1500-1600 kcal, or 1400-1500 kcal. In some embodiments, men were provided a diet of 1500-1800 kcal, or 1400-1800 kcal, or 1800-2200 kcal, or 1600-2000 kcal. In a preferred embodiment, men were provided a diet of 1800 kcal.

In stage 2 (subjects were subjected to low calorie “liquid diet” for 120 days), enrolled women were recommended a diet of 800-1200 kcal. In some embodiments, women were provided a diet of 1100-1200 kcal, or 1000-1100 kcal, or 900-1000 kcal, or 800-900 kcal, or 900-1100 kcal. In a preferred embodiment, women were provided a diet of 1000 kcal per day.

In stage 2 (subjects were subjected to low calorie “liquid diet” for 120 days), enrolled men were recommended a diet of 1000-1500 kcal. In some embodiments, men were provided a diet of 1400-1500 kcal, or 1300-1400 kcal, or 1200-1300 kcal, or 1100-1200 kcal, or 1000-1100, or 1100-1300 kcal. In a preferred embodiment, men were provided a diet of 1200 kcal per day.

According to some embodiments, a low calorie diet and a low calorie liquid diet refers to a low fat or low carbohydrate diet or both.

Subjects, who lost >3% weight after being on a recommended diet in stage 1, were classified as Group A. In stage 2 (after the first 4 months, another about 4 months) all subjects who lost <3% weight in stage 1 were recommended a liquid diet of 1000 kcal (women) or 1200 kcal (men). Once on liquid diet, subjects who lost >5% of total body weight in an early stage were classified as Group B (early responders), and those who lost the same amount of weight, but at a later stage were categorized in Group C (late responders). Subjects, who did not respond to either stages (I or II), were classified as Group D (non-responders).

According to some embodiments, the Group B early responders responded to liquid diet between 20-30 days, or 31-40 days, or 41-50 days, or 51-60 days, or 61-70 days, or 71-80 days, or 81-90 days, or 91-100 days, or 101-110 days, or 111-120 days. In some preferred embodiments, the Group B early responders responded between 20-120 days, or 20-60 days, or 30-60 days, or 30-120 days, or 60-120 days.

According to some embodiments, the Group C late responders responded to liquid diet between 120-130 days, or 131-140 days, or 141-150 days, or 151-160 days, or 161-170 days, or 171-180 days, or 181-190 days, or 191-200 days, or 201-210 days, or 211-220 days, or 221-230 days, or 231-240 days, or 241-250 days, or 251-260 days, or 261-270 days, or 271-280 days, or 281-290 days, or 291-300 days, or 301-310 days, or 311-320 days, or 321-330 days, or 331-340 days, or 341-350 days, or 351-360 days, or 361-370 days. In some preferred embodiments, the Group C late responders respond between 121-190 days, or 121-360 days, or 121-370 days, or 121-180 days, or 121-220 days, or 121-160 days, or 160-200 days, or 160-180 days, or 160-220 days, or 180-220 days, or 180-370 days.

Nutrition categories are generally classified on the basis of the amount of macronutrients (i.e., fat, carbohydrates, protein) recommended for a subject based on that subject's metabolic genotype. The primary goal of selecting an appropriate therapeutic/dietary regimen or lifestyle recommendation for a subject is to pair a subject's metabolic genotype with the nutrition category to which that subject is most likely to be responsive. A nutrition category is generally expressed in terms of the relative amounts of macronutrients suggested for a subject's diet or in terms of calories restrictions (e.g., restricting the total number of calories a subject receives and/or restricting the number of calories a subject receives from a particular macronutrient). For example, nutrition categories may include, but are not limited to, 1) low fat, low carbohydrate diets; 2) low fat diets, or 3) low carbohydrate diets. Alternatively, nutrition categories may be classified on the basis of the restrictiveness of certain macronutrients recommended for a subject based on that subject's metabolic genotype. For example, nutrition categories may be expressed as 1) balanced or calorie restricted diets; 2) fat restrictive diets, or 3) carbohydrate restrictive diets.

Subjects with a metabolic genotype that is responsive to fat restriction or low fat diet tend to absorb more dietary fat into the body and have a slower metabolism. They have a greater tendency for weight gain. Clinical studies have shown these subjects have an easier time reaching a healthy body weight by decreasing total dietary fat. They may have greater success losing weight by following a reduced fat and/or reduced calorie diet. In addition, they benefit from replacing saturated fats with monounsaturated fats within a reduced calorie diet. Clinical studies have also shown these same dietary modifications improve the body's ability to metabolize sugars and fats.

Subjects with a metabolic genotype that is responsive to carbohydrate restriction or low carbohydrate diet tend to be more sensitive to weight gain from excessive carbohydrate intake. They may have greater success losing weight by reducing carbohydrates within a reduced calorie diet. Subjects with this genetic pattern are prone to obesity and have difficulty with blood sugar regulation if their daily carbohydrate intake is high, such as where the daily carbohydrate intake exceeds, for example, about 49% of total calories. Carbohydrate reduction has been shown to optimize blood sugar regulation and reduce risk of further weight gain. If they have high saturated and low monounsaturated fats in their diet, risk for weight gain and elevated blood sugar increases. While limiting total calories, these subjects may benefit from restricting total carbohydrate intake and shifting the fat composition of their diet to monounsaturated fats (e.g., a diet low in saturated fat and low in carbohydrate).

Subjects with a metabolic genotype that is responsive to a balance of fat and carbohydrate show no consistent need for a low fat or low carbohydrate diet. In these subjects key biomarkers, such as body weight, body fat, and plasma lipid profile, respond well to a diet balanced in fat and carbohydrate. For subjects with this genetic pattern who are interested in losing weight, a balanced diet restricted in calories has been found to promote weight loss and a decrease in body fat, wherein the fat content of a subject is reduced irrespective of the body weight (lean body mass). Body fat may be measured by methods well known in the art. A preferred method is DEXA (Dual Energy X-ray Absorptiometry)—a technology that is very accurate and precise. DEXA is based on a three-compartment model that divides the body into total body mineral, fat-free soft (lean) mass, and fat tissue mass. This technique is based on the assumption that bone mineral content is directly proportional to the amount of photon energy absorbed by the bone being studied. Other methods for measurement of body fat includes, but not limited to: NIR (Near Infrared Interactance); MRI (Magnetic Resonance Imaging); TOBEC (Total Body Electrical Conductivity); CT (Compound Tomography); BOD POD (Air Displacement); BIA (Bioelectrical Impedance).

A low fat diet refers to a diet that provides between about 10% to less than about 40% of total calories from fat. According to some embodiments, a low fat diet refers to a diet that provides no more than about 35 percent (e.g., no more than about 19%, 21%, 23%, 22%, 24%, 26%, 28%, 33%, etc) of total calories from fat. According to some embodiments, a low fat diet refers to a diet that provides no more than about 30 percent of total calories from fat. According to some embodiments, a low fat diet refers to a diet that provides no more than about 25 percent of total calories from fat. According to some embodiments, a low fat diet refers to a diet that provides no more than about 20 percent of total calories from fat. According to some embodiments, a low fat diet refers to a diet that provides no more than about 15 percent of total calories from fat. According to some embodiments, a low fat diet refers to a diet that provides no more than about 10 percent of total calories from fat.

According to some embodiments, a low fat diet refers to a diet that is between about 10 grams and about 60 grams of fat per day. According to some embodiments, a low fat diet refers to a diet that is less than about 50 grams (e.g., less than about 10, 25, 35, 45, etc) grams of fat per day. According to some embodiments, a low fat diet refers to a diet that is less than about 40 grams of fat per day. According to some embodiments, a low fat diet refers to a diet that is less than about 30 grams of fat per day. According to some embodiments, a low fat diet refers to a diet that is less than about 20 grams of fat per day.

Fats contain both saturated and unsaturated (monounsaturated and polyunsaturated) fatty acids. According to some embodiments, reducing saturated fat to less than 10 percent of calories is a diet low in saturated fat. According to some embodiments, reducing saturated fat to less than 15 percent of calories is a diet low in saturated fat. According to some embodiments, reducing saturated fat to less than 20 percent of calories is a diet low in saturated fat.

A low carbohydrate (CHO) diet refers to a diet that provides between about 20% to less than about 50% of total calories from carbohydrates. According to some embodiments, a low carbohydrate (CHO) diet refers to a diet that provides no more than about 50 percent (e.g., no more than about 20%, 25%, 30%, 35%, 40%, 45%, etc) of total calories from carbohydrates. According to some embodiments, a low carbohydrate diet refers to a diet that provides no more than about 45 percent of total calories from carbohydrates. According to some embodiments, a low carbohydrate diet refers to a diet that provides no more than about 40 percent of total calories from carbohydrates. According to some embodiments, a low carbohydrate diet refers to a diet that provides no more than about 35 percent of total calories from carbohydrates. According to some embodiments, a low carbohydrate diet refers to a diet that provides no more than about 30 percent of total calories from carbohydrates. According to some embodiments, a low carbohydrate diet refers to a diet that provides no more than about 25 percent of total calories from carbohydrates. According to some embodiments, a low carbohydrate diet refers to a diet that provides no more than about 20 percent of total calories from carbohydrates.

A low carbohydrate (CHO) diet may refer to a diet that restricts the amount of grams of carbohydrate in a diet such as a diet of from about 20 to about 250 grams of carbohydrates per day. According to some embodiments, a low carbohydrate diet comprises no more than about 220 (e.g., no more than about 40, 70, 90, 110, 130, 180, 210, etc) grams of carbohydrates per day. According to some embodiments, a low carbohydrate diet comprises no more than about 200 grams of carbohydrates per day. According to some embodiments, a low carbohydrate diet comprises no more than about 180 grams of carbohydrates per day. According to some embodiments, a low carbohydrate diet comprises no more than about 150 grams of carbohydrates per day. According to some embodiments, a low carbohydrate diet comprises no more than about 130 grams of carbohydrates per day. According to some embodiments, a low carbohydrate diet comprises no more than about 100 grams of carbohydrates per day. According to some embodiments, a low carbohydrate diet comprises no more than about 75 grams of carbohydrates per day.

A low carbohydrate diet may also be referred to as a low-glycemic-load diet, and a high carbohydrate diet may also be referred to as high-glycemic-load diet. Accoriding to some embodiments, a high-glycemic (HG or high CHO) diet and a low-glycemic (LG or low CHO) diet may both be designed to promote calorie restriction (CR), while differing in the ratio of macronutrients. That is, the diets may differ in the ratio of macronutrients (for example, HG: 60% carbohydrate, 20% fat, and 20% protein; and LG: 40% carbohydrate, 30% fat, and 30% protein). The carbohydrate sources in the LG diet preferably have a lower glycemic index (GI) per published GIs of different carbohydrate sources (see e.g., International table of glycemic index and glycemic load values: 2002. Am J Clin Nutr 2002; 76:5-56, incorporated herein by reference in its entirety).

Examples of food used for a HG diet include, but are not limited to, the following: candied sweet potatoes; carrots; chicken and pea casserole; chef salad; chicken and rice; couscous; english muffins and bagels; jelly; jasmine rice; Lactose-free skim milk (Lactaid; McNeil Nutritionals, LLC, Fort Washington, Pa.); oatmeal; pizza; sugar cookies and graham crackers; shepherd's pie with mashed potatoes; sweet and sour chicken; turkey with cranberry sauce; tuna sandwich; waffles; and yogurt with added fruit—canned pears, peaches, figs, pineapple, oranges, and bananas. Examples of food used for a LG diet include, but are not limited to, the following: baked chicken; bean and barley stew; bulgur and beans; broccoli and beans; cottage cheese, low-fat; curried lentils; fish; fruit: oranges, grapefruit, plums, pears, apples, and berries; flaxseed cookies; green salad; Kashi (Kashi, La Jolla, Calif.) and Muesli cereal (Kellogg's Co, Battle Creek, Mich.); lentils with tomato sauce; nuts; pumpernickel bread; salisbury steak; skim milk; tomato cucumber bean salad; wheat berry salad; and yogurt.

According to some embodiments, both the HG and LG diets may be designed with features to promote calorie restriction, including, but not limited to, the following: meeting Dietary Reference Intakes (DRIs) for dietary fiber (Institute of Medicine. Dietary reference intakes: energy, carbohydrate, fiber, fat, fatty acids, cholesterol, protein, and amino acids. Vol 5. Washington, D.C.: The National Academy Press, 2002:1-114, incorporated herein by reference in its entirety); limited inclusion of high-energy-density foods (as defined in Rolls et al., J Am Diet Assoc 2005; 105(suppl):S98-103, incorporated herein by reference in its entirety); limited liquid calories (as defined in Mattes, Physiol Behav 1996; 59: 179-87, incorporated herein by reference in its entirety); and a relatively high variety of low-energy-density foods (e.g., fruit and vegetables), and a relatively low variety of high-energy-dense foods (as defined in McCrory et al., Am J Clin Nutr 1999; 69:440-7, incorporated herein by reference in its entirety).

A calorie restricted (CR) diet or balanced diet refers to a diet that is restricts total calories consumed to below a subject's weight maintenance level (WML), regardless of any preference for a macronutrient. A balanced diet or calorie restricted diet seeks to reduce the overall caloric intake of a subject by, for example, reducing the total caloric intake of a subject to below that subject's WML without a particular focus on restricting the calories consumed from any particular macronutrient. For example, calorie restricted diet may contain the range of current dietary recommendations for healthful macronutrient ranges and containing the Dietary Reference Intakes (DRIs) of micronutrients and essential fatty acids at 10-50% (e.g., 10% 15%, 20%, 25%, 30%, 35%, 40%, 45%, or 50%) calorie restriction (CR) relative to baseline energy requirements. Thus, according to some embodiments, a balanced diet may be expressed as a percentage of a subject's WML. For example, a balanced diet is a diet that comprises a total caloric intake of between about 50% to about 100% WML. According to some embodiments, a balanced diet is a diet that comprises a total caloric intake of less than 100% (e.g., less than about 99%, 97%, 95%, 90%, 85%, 80%, 75%, 70%, 65%, 60%, 55%) of WML. Within this framework, a balanced diet achieves a healthy or desired balance of macronutrients in the diet and may be: low fat; low saturated fat; low carbohydrate; low fat and low carbohydrate; or low saturated fat and low carbohydrate. For example, a diet may be a low fat, calorie restricted diet (where low fat has the meaning as provided hereinabove). A diet may be a low carbohydrate, calorie restricted diet (where low carbohydrate has the meaning as provided hereinabove). A diet may be a balanced, calorie restricted diet (e.g., relative portions of macronutrients may vary where the total calories consumed is below the WML).

According to some embodiments, a low-carb diet (Carb: 45%, Protein: 20%, and Fat: 35%) comprises any of: Atkins diet, Glycemic Impact Diet, South Beach Diet, Sugar Busters Diet, and/or Zone diet.

According to some embodiments, a low-fat diet (Carb: 65%, Protein: 15%, Fat:20%) comprises any of: Life Choice Diet (Ornish Diet), Pritikin Diet, and/or other heart healthy diets available in the market.

According to some embodiments, a balanced diet (Carb: 55%, Protein: 20%, Fat: 25%) comprises any of: Best Life Diet, Mediterranean Diet, Sonoma Diet, Volumetrics Eating Diet, Weight Watchers Diet.

Other low carbohydrate, low fat, balanced diet or calorie restricted diets are well known in the art, thus can be recommended to a subject depending on the subject's metabolic genotype and predicted response to calorie restricted or other diet types.

Gain or loss of weight depends on a balance between calories consumed and calories expended. When the amount of calories consumed is greater than the number of calories expended, weight gain may occur. In contrast, if calories consumed are less than the number of calories expended, weight loss may occur. A subject's WML refers to the total caloric intake a subject needs to consume in order to maintain current body weight. A subject's WML may be determined or calculated using any method known in the art. WML is often expressed as total daily energy expenditure (TDEE), total energy expenditure (TEE; as defined by Das et al. Am J Clin Nutr. 2007 April; 85(4):1023-30, incorporated by reference herein in its entirety), or estimated energy requirements (EER). While the meaning of TDEE, TEE, and EER as used in the art may have technical distinctions reflecting the manner in which a subject's weight maintenance level is calculated, these terms may be used interchangeably in their general sense while maintaining their technical distinctions. WML may be calculated using any method used in the art (e.g., TDEE, TEE or EER) to determine a subject's WML.

On average, for females in the U.S. the WML is between 2000-2100 calories per day. Males average a higher WML at 2700-2900 calories per day. A preferred method for calculating TDEE is by using the Harris-Benedict calculation or Katch-McArdle formula, which are well known to those of ordinary skill in the art. Briefly, the Harris-Benedict formula first determines and subject's basal metabolic rate (BMR), which is then adjusted base for activity level to give a subject's TDEE. For example, BMR for females may be calculated according to the following formula: BMRf=65.51 +(9.563×kg)+(1.850×cm)−(4.676×age). BMR for males may be calculated according to the following formula: BMRₘ=66.5 +(13.75×kg)+(5.003×cm)-(6.775×age). The BMR is then adjusted by multiplying BMR by a multiplier assigned to a particular activity level. The table below provides examples of such multipliers. The result is a subject's TDEE.

The Katch & McArdle formula is based on a subject's lean body mass (LBM). For example, BMR is calculated according to the following formula: BMR (men and women)=370 +(21.6×lean mass in kg). Since the Katch-McArdle formula accounts for LBM, this single formula applies equally to both men and women. TDEE is then determined using the activity multipliers as used in the Harris-Benedict calculation (in the table above).

TABLE 1
TDEE

| | Females | Males |
|---|---|---|
| Little or no exercise | BMR_{f} × 1.2 | BMRₘ × 1.2 |
| Light exercise | BMR_{f} × 1.375 | BMRₘ × 1.375 |
| Moderate exercise | BMR_{f} × 1.55 | BMRₘ × 1.55 |
| Heavy exercise | BMR_{f} × 1.725 | BMRₘ × 1.725 |
| Very heavy exercise | BMR_{f} × 1.9 | BMRₘ × 1.9 |

Exercise Categories

Exercise categories are generally classified on the basis of how responsive a subject is to exercise given their metabolic genotype. For example, a subject may be responsive to light exercise, moderate exercise, heavy exercise, or very heavy exercise.

Subjects with a metabolic genotype that is responsive to exercise are able to effectively break down body fat in response to physical activity. They tend to respond to exercise with significant weight loss and are more likely to maintain that weight loss. Subjects fall into this category if they are responsive to light or moderate exercise.

Subjects with a metabolic genotype that is less responsive to exercise are less able to break down body fat for energy in response to exercise than those with the alternative genetic pattern. They tend to lose less weight and body fat than expected with moderate exercise. These subjects require more exercise to activate the breakdown of body fat for energy and weight loss. They must also maintain a consistent exercise program to keep the weight off.

Light activity generally refers to a subject that exercises (engages in an active workout or sports) 1-3 days per week. Moderate activity generally refers to a subject that exercises (engages in an active workout or sports) 3-5 days per week. High activity generally refers to a subject that exercises (engages in an active workout or sports) 6-7 days per week. Very high or extreme activity generally refers to a subject that exercises (engages in an active workout or sports) on average of more than once a day (e.g., two times per day). Regular exercise refers to activity that is at least light exercise or at least moderate exercise.

More accurately, activity level may be expressed in terms of a percentage over BMR. For example, the multipliers of the Harris-Benedict or Katch-McArdle formulas may be used as a basis to define an activity level. Accordingly, light exercise refers to a recommended activity level designed to increase a subject's TDEE to about 125% of BMR (i.e., about a 25% increase) to less than about 140% (e.g., about 128%, 130%, 133%, 135%, 137.5%, etc) of BMR. Moderate exercise refers to a recommended activity level designed to increase a subject's TDEE to about 140% of BMR to less than about 160% (e.g., about 142%, 145%, 150%, 155%, 158%, etc) of BMR. Heavy exercise refers to a recommended activity level designed to increase a subject's TDEE to about 160% of BMR to less than about 180% (e.g., about 162%, 165%, 170%, 172.5%, 175%, 178%, etc) of BMR. Very heavy or extreme exercise refers to a recommended activity level designed to increase a subject's TDEE to about 180% of BMR to more than about 210% (e.g., about 182%, 185%, 190%, 195%, 200%, etc) of BMR.

A subject's metabolic genotype may fall into a single nutrition category and a single exercise category. Thus, according to some embodiments, a subject will be classified into a nutrition category and exercise category based on their metabolic genotype. For example, a subject may be classified into one of the following six categories: 1) Responsive to Fat Restriction and Responsive to Exercise; 2) Responsive to Fat Restriction and Less Responsive to Exercise; 3) Responsive to Carbohydrate Restriction and Responsive to Exercise; 4) Responsive to Carbohydrate Restriction and Less Responsive to Exercise; 5) Balance of Fat and Carbohydrate and Responsive to Exercise; and 6) Balance of Fat and Carbohydrate and Less Responsive to Exercise.

1) Responsive to Fat Restriction and Responsive to Exercise: Subjects with this genetic pattern absorb more dietary fat into the body and have a slower metabolism. They have a greater tendency for weight gain. Clinical studies have shown these subjects have an easier time reaching a healthy body weight by decreasing total dietary fat. They may have greater success losing weight by following a reduced fat, reduced calorie diet. In addition, they benefit from replacing saturated fats with monounsaturated fats within a reduced calorie diet. Clinical studies have also shown these same dietary modifications improve the body's ability to metabolize sugars and fats.

Subjects with this genetic pattern are able to effectively breakdown body fat in response to physical activity. They tend to respond to exercise with significant weight loss and are more likely to maintain that weight loss. Such subjects may benefit from any level of increased activity such as at least light exercise or at least moderate exercise.

2) Responsive to Fat Restriction and Less Responsive to Exercise Subjects with this genetic pattern absorb more dietary fat into the body and have a slower metabolism. They have a greater tendency for weight gain. Clinical studies have shown these subjects have an easier time reaching a healthy body weight by decreasing total dietary fat. They may have greater success losing weight by following a reduced fat, reduced calorie diet. In addition, they benefit from replacing saturated fats with monounsaturated fats within a reduced calorie diet. Clinical studies have also shown these same dietary modifications improve the body's ability to metabolize sugars and fats.

Subjects with this genetic pattern are less able to breakdown body fat for energy in response to exercise than those with the alternative genetic pattern. They tend to lose less weight and body fat than expected with moderate exercise. These subjects require more exercise to activate the breakdown of body fat for energy and weight loss. They must also maintain a consistent exercise program to keep the weight off.

3) Responsive to Carbohydrate Restriction and Responsive to Exercise Subjects with this genetic pattern are more sensitive to weight gain from excessive carbohydrate intake. They may have greater success losing weight by reducing carbohydrates within a reduced calorie diet. Subjects with this genetic pattern are prone to obesity and have difficulty with blood sugar regulation if their daily carbohydrate intake exceeds 49% of total calories. Carbohydrate reduction has been shown to optimize blood sugar regulation and reduce risk of further weight gain. If they have high saturated and low monounsaturated fats in their diet, risk for weight gain and elevated blood sugar increases. While limiting total calories, these subjects may benefit from restricting total carbohydrate intake and shifting the fat composition of their diet to monounsaturated fats.

Subjects with this genetic pattern are able to effectively breakdown body fat in response to physical activity. They tend to respond to exercise with significant weight loss and are more likely to maintain that weight loss.

4) Responsive to Carbohydrate Restriction and Less Responsive to Exercise Subjects with this genetic pattern are more sensitive to weight gain from excessive carbohydrate intake. They may have greater success losing weight by reducing carbohydrates within a reduced calorie diet. Subjects with this genetic pattern are prone to obesity and have difficulty with blood sugar regulation if their daily carbohydrate intake exceeds 49% of total calories. Carbohydrate reduction has been shown to optimize blood sugar regulation and reduce risk of further weight gain. If they have high saturated and low monounsaturated fats in their diet, risk for weight gain and elevated blood sugar increases. While limiting total calories, these subjects may benefit from restricting total carbohydrate intake and shifting the fat composition of their diet to monounsaturated fats.

Subjects with this genetic pattern are less able to breakdown body fat for energy in response to exercise than those with the alternative genetic pattern. They tend to lose less weight and body fat than expected with moderate exercise. These subjects require more exercise to activate the breakdown of body fat for energy and weight loss. They must also maintain a consistent exercise program to keep the weight off.

5) Balance of Fat and Carbohydrate and Responsive to Exercise Subjects with this genetic pattern show no consistent need for a low fat or low carbohydrate diet. In these subjects key biomarkers, such as body weight, body fat, and plasma lipid profile, respond well to a diet balanced in fat and carbohydrate. For subjects with this genetic pattern who are interested in losing weight, a balanced diet restricted in calories has been found to promote weight loss and a decrease in body fat.

Subjects with this genetic pattern are able to effectively breakdown body fat in response to physical activity. They tend to respond to exercise with significant weight loss and are more likely to maintain that weight loss.

6) Balance of Fat and Carbohydrate and Less Responsive to Exercise Subjects with this genetic pattern show no consistent need for a low fat or low carbohydrate diet. In these subjects key biomarkers, such as body weight, body fat, and plasma lipid profile, respond well to a diet balanced in fat and carbohydrate. For subjects with this genetic pattern who are interested in losing weight, a balanced diet restricted in calories has been found to promote weight loss and a decrease in body fat.

Subjects with this genetic pattern are less able to breakdown body fat for energy in response to exercise than those with the alternative genetic pattern. They tend to lose less weight and body fat than expected with moderate exercise. These subjects require more exercise to activate the breakdown of body fat for energy and weight loss. They must also maintain a consistent exercise program to keep the weight off.

According to some embodiments, a normal exercise routing comprises: 2.5 hours (150 minutes) of moderate-intensity activity per week (Moderate-intensity activities are defined as 3.0 to 5.9 METs).

According to some embodiments, a vigorous exercise routine comprises: greater than 13 METs per week of vigorous intensity activities (Vigorous intensity activities are defined as 6 METs or greater). 1 MET is equal to 1 calorie/kg body mass/hour. The total kcal expended by a subject=MET value of activity×body weight in kg×time in hours.

In addition to the nutritional and exercise recommendations, the personalized therapeutic/dietary regimen may also include recommendation for dietary supplements, food supplements, or nutraceuticals. A “nutraceutical” is any functional food that provides an additional benefit other than its nutritional benefit. This category may include nutritional drinks, diet drinks (e.g., Slimfast™ and the like) as well as sports herbal and other fortified beverages.

Detection of Alleles

Allelic patterns, polymorphism patterns, or haplotype patterns can be identified by detecting any of the component alleles using any of a variety of available techniques, including: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments generated by endonuclease digestion). The allele can optionally be subjected to an amplification step prior to performance of the detection step. Preferred amplification methods are selected from the group consisting of: the polymerase chain reaction (PCR), the ligase chain reaction (LCR), strand displacement amplification (SDA), cloning, and variations of the above (e.g. RT-PCR and allele specific amplification). Oligonucleotides necessary for amplification may be selected, for example, from within the metabolic gene loci, either flanking the marker of interest (as required for PCR amplification) or directly overlapping the marker (as in allele specific oligonucleotide (ASO) hybridization). In a particularly preferred embodiment, the sample is hybridized with a set of primers, which hybridize 5′ and 3′ in a sense or antisense sequence to the vascular disease associated allele, and is subjected to a PCR amplification.

An allele may also be detected indirectly, e.g. by analyzing the protein product encoded by the DNA. For example, where the marker in question results in the translation of a mutant protein, the protein can be detected by any of a variety of protein detection methods. Such methods include immunodetection and biochemical tests, such as size fractionation, where the protein has a change in apparent molecular weight either through truncation, elongation, altered folding or altered post-translational modifications.

A general guideline for designing primers for amplification of unique human chromosomal genomic sequences is that they possess a melting temperature of at least about 50° C., wherein an approximate melting temperature can be estimated using the formula Tₘₑₗₜ=[2×(# of A or T)+4×(# of G or C)].

Many methods are available for detecting specific alleles at human polymorphic loci. The preferred method for detecting a specific polymorphic allele will depend, in part, upon the molecular nature of the polymorphism. For example, the various allelic forms of the polymorphic locus may differ by a single base-pair of the DNA. Such single nucleotide polymorphisms (or SNPs) are major contributors to genetic variation, comprising some 80% of all known polymorphisms, and their density in the human genome is estimated to be on average 1 per 1,000 base pairs. SNPs are most frequently biallelic-occurring in only two different forms (although up to four different forms of an SNP, corresponding to the four different nucleotide bases occurring in DNA, are theoretically possible). Nevertheless, SNPs are mutationally more stable than other polymorphisms, making them suitable for association studies in which linkage disequilibrium between markers and an unknown variant is used to map disease-causing mutations. In addition, because SNPs typically have only two alleles, they can be genotyped by a simple plus/minus assay rather than a length measurement, making them more amenable to automation.

A variety of methods are available for detecting the presence of a particular single nucleotide polymorphic allele in a subject. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Publication No. WO92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Publication No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

For mutations that produce premature termination of protein translation, the protein truncation test (PTT) offers an efficient diagnostic approach (Roest, et. al., (1993) Hum. Mol. Genet. 2:1719-2 1; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

Any cell type or tissue may be utilized to obtain nucleic acid samples for use in the diagnostics described herein. In a preferred embodiment, the DNA sample is obtained from a bodily fluid, e.g, blood, obtained by known techniques (e.g. venipuncture) or saliva. Alternatively, nucleic acid tests can be performed on dry samples (e.g. hair or skin). When using RNA or protein, the cells or tissues that may be utilized must express a metabolic gene of interest.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

A preferred detection method is allele specific hybridization using probes overlapping a region of at least one allele of a metabolic gene or haplotype and having about 5, 10, 20, 25, or 30 nucleotides around the mutation or polymorphic region. In a preferred embodiment of the invention, several probes capable of hybridizing specifically to other allelic variants of key metabolic genes are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P. M. et al., 1988, Bio/Technology 6:1197).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one allele of a metabolic gene or haplotype under conditions such that hybridization and amplification of the allele occurs, and (iv) detecting the amplification product. These detection schemes are especially useful for the detection of nucleic acid molecules if such molecules are present in very low numbers.

In a preferred embodiment of the subject assay, the allele of a metabolic gene or haplotype is identified by alterations in restriction enzyme cleavage patterns. For example, sample and control DNA is isolated, amplified (optionally), digested with one or more restriction endonucleases, and fragment length sizes are determined by gel electrophoresis.

In yet another embodiment, any of a variety-of sequencing reactions known in the art can be used to directly sequence the allele. Exemplary sequencing reactions include those based on techniques developed by Maxim and Gilbert ((1977) Proc. Natl. Acad Sci USA 74:560) or Sanger (Sanger et al (1977) Proc. Nat. Acad. Sci. USA 74:5463). It is also contemplated that any of a variety of automated sequencing procedures may be utilized when performing the subject assays (see, for example Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl. Acad Sci USA 85:4397; and Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an allele of a metabolic gene locus haplotype is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify a metabolic gene locus allele. For example, single strand conformation polymorphism (SSCP) may be used to detect differences in electrophoretic mobility between mutant and wild type nucleic acids (Orita et al. (1989) Proc Natl. Acad. Sci. USA 86:2766, see also Cotton (1993) Mutat Res 285:125-144; and Hayashi (1992) Genet Anal Tech Appl 9:73-79). Single-stranded DNA fragments of sample and control metabolif locus alleles are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of alleles in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl. Acad. Sci. USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 1 1:238). In addition it may be desirable to introduce a novel restriction site in the region of the mutation to create cleavage-based detection (Gasparini et al (1992) Mol. Cell. Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to detect alleles of a metabolic gene locus haplotype. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Another embodiment of the invention is directed to kits for detecting a predisposition for responsiveness to certain diets and/or activity levels. This kit may contain one or more oligonucleotides, including 5′ and 3′ oligonucleotides that hybridize 5′ and 3′ to at least one allele of a metabolic gene locus or haplotype. PCR amplification oligonucleotides should hybridize between 25 and 2500 base pairs apart, preferably between about 100 and about 500 bases apart, in order to produce a PCR product of convenient size for subsequent analysis.

Particularly preferred primers for use in the diagnostic method of the invention include SEQ ID NO: 1-25.

TABLE 2

| | | | |
|---|---|---|---|
| SNP | SEQ ID NO: | Purpose | Sequence |

| | | | |
|---|---|---|---|
| IL1RN rs315952 | SEQ ID NO: 1 | PCR | GCCTCAGCTCTCACCTGCCCATCTT |
| T > C | | | TTG |
| | SEQ ID NO: 2 | PCR | AGGCAGCATGGAGGCTGGTCAGTT |
| | | | GAA |
| | SEQ ID NO: 3 | SBE | GACAAGCGCTTCGCCTTCATCCGCT |
| | | | CAGACAG |
| IL1A (+4845) | SEQ ID NO: 4 | PCR | AGAAAACCAGTTCTGCTGACTGGG |
| rs17561 G > T | | | TGA |
| | SEQ ID NO: 5 | PCR | TGGCTTGGGATTTTTATGGGGGTGC |
| | | | TG |
| | SEQ ID NO: 6 | SBE | CATGGTTTTAGAAATCATCAAGCCT |
| | | | AGGTCA |
| IL1B (+3954) | SEQ ID NO: 7 | PCR | TATGCTCAGGTGTCCTCCAAGAAAT |
| rs1143634 | | | CA |
| C > T | SEQ ID NO: 8 | PCR | TTGTTGCTCCATATCCTGTCCCTGG |
| | | | AG |
| | SEQ ID NO: 9 | SBE | ACATGTGCTCCACATTTCAGAACCT |
| | | | ATCTTCTT |
| IL1B (−511) | SEQ ID NO: 10 | PCR | CCTGACAATCGTTGTGCAGTTGATG |
| rs16944 | | | TCCA |
| C > T | SEQ ID NO: 11 | PCR | CAGCACCTGGTCTTGCAGGGTTGTG |
| | | | TG |
| | SEQ ID NO: 12 | SBE | GTGTGGGTCTCTACCTTGGGTGCTG |
| | | | TTCTCTGCCTC |
| IL1B (−3737) | SEQ ID NO: 13 | PCR | ACATCAGGGAAAAGCCATTG |
| rs4848306 | SEQ ID NO: 14 | PCR | TGGGAATGGGCACTATGATT |
| C > T | SEQ ID NO: 15 | SBE | GATTGGGGACATGCAGAGTCCAAG |
| | | | G |
| ADRB2 (Q27E) | SEQ ID NO: 16 | PCR | GCCCCTAGCACCCGACAAGCTGAG |
| rs1042714 | | | TGT |
| | SEQ ID NO: 17 | PCR | CCAGGCCCATGACCAGATCAGCAC |
| | | | AG |
| | SEQ ID NO: 18 | SBE | AGCCATGCGCCGGACCACGACGTC |
| | | | ACGCAG |
| ADRB2 (R16G) | SEQ ID NO: 19 | PCR | GCCCCTAGCACCCGACAAGCTGAG |
| rs1042713 | | | TGT |
| | SEQ ID NO: 20 | PCR | CCAGGCCCATGACCAGATCAGCAC |
| | | | AG |
| | SEQ ID NO: 21 | SBE | AACGGCAGCGCCTTCTTGCTGGCAC |
| | | | CCAAT |
| ADRB3 | SEQ ID NO: 22 | PCR | AAGCGTCGCTACTCCTCCCCCAAGA |
| rs4994 | | | GC |
| | SEQ ID NO: 23 | PCR | GTCACACACAGCACGTCCACCGAG |
| | | | GTC |
| | SEQ ID NO: 25 | SBE | GGGAGGCAACCTGCTGGTCATCGT |
| | | | GGCCATCGCC |
| FABP2 | SEQ ID NO: 26 | PCR | TGTTCTTGTGCAAAGGCAATGCTAC |
| rs1799883 | | | CG |
| | SEQ ID NO: 27 | PCR | TCTTACCCTGAGTTCAGTTCCGTCT |
| | | | GC |
| | SEQ ID NO: 28 | SBE | GAAGGAAATAAATTCACAGTCAAA |
| | | | GAATCAAGC |
| MCR4 | SEQ ID NO: 29 | PCR | GGAGACTGGCAAAGCAGAGTTTTT |
| rs12970134 | | | GCGAGA |
| | SEQ ID NO: 30 | PCR | GGAGACATGCTTGCCCTGCTAGGTT |
| | | | GGTC |
| | SEQ ID NO: 31 | SBE | CTGATACTGACTCTTACCAAACAAA |
| | | | GCATGA |
| MCR4 | SEQ ID NO: 32 | PCR | TGTGGGTACTGGACACAGACAGGT |
| rs477181 | | | GTTCC |
| | SEQ ID NO: 33 | PCR | TCATTAATTGTTTGGCTCAATGGGT |
| | | | CATC |
| | SEQ ID NO: 34 | SBE | GGCAGAGATAATAGAAGGAATCAT |
| | | | AGTGTCATC |
| MCR4 | SEQ ID NO: 35 | PCR | TGTGGCAATAGCCAAGAACAAGAA |
| rs2229616 | | | TCTGC |
| | SEQ ID NO: 36 | PCR | GTCCACTGCAATTGAAAGCAGGCT |
| | | | GCAAA |
| | SEQ ID NO: 37 | SBE | CCGTATCTGTACTGTTTAATAGGGT |
| | | | GATGA |
| MCR4 | SEQ ID NO: 38 | PCR | CCCATGGGAGATCAATCTTTTCTTC |
| rs502933 | | | AGAT |
| | SEQ ID NO: 39 | PCR | GCTCATCATCAATATCAGAGCCAG |
| | | | AGTGTG |
| | SEQ ID NO: 40 | SBE | GGTTACTTAGTTACGAAGCCAATAC |
| | | | CAACCTAT |
| PPARG | SEQ ID NO: 41 | PCR | TGCCAGCCAATTCAAGCCCAGTCCT |
| rs1801282 | | | TT |
| | SEQ ID NO: 42 | PCR | ACACAACCTGGAAGACAAACTACA |
| | | | AGAGCAA |
| | SEQ ID NO: 43 | SBE | GACAGTGTATCAGTGAAGGAATCG |
| | | | CTTTCTG |
| IL1B (+3877) | SEQ ID NO: 44 | PCR | TTAGCCACCCCACTCCCAGCTTCAT |
| rs1143633 | | | CC |
| | SEQ ID NO: 45 | PCR | CAGGTGCATCGTGCACATAAGCCT |
| | | | CGT |
| | SEQ ID NO: 46 | SBE | GCTCAGGTGTCCTCCAAGAAATCA |
| | | | AATTTTGCC |
| IL1A | SEQ ID NO: 47 | PCR | TCCCAGTTTTGCAGATGAGGCAATG |
| rs10496444 | | | GA |
| | SEQ ID NO: 48 | PCR | AAGCCCTGGGGAATGAGGTGGCAA |
| | | | AGA |
| | SEQ ID NO: 49 | SBE | GCAAATCTAACTCTTCAAGCTAACA |
| | | | CATAGCAA |
| IL1B | SEQ ID NO: 50 | PCR | AAATCAGAAGGCTGCTTGGA |
| rs1143623 | SEQ ID NO: 51 | PCR | ATGGGTGAATGGGAATTTGA |
| | SEQ ID NO: 52 | SBE | CTCAAATACTTGCACAGAGGCTCA |
| | | | CTCCCTTG |
| IL1B | SEQ ID NO: 53 | PCR | AACTGCGTGCAACCTTCAATCCTGC |
| rs1143643 | | | TG |
| | SEQ ID NO: 54 | PCR | TGTGGGGCAAGGGACAAAGATGCT |
| | | | ATGG |
| | SEQ ID NO: 55 | SBE | GAAGAAGGGCTCTTTTAATAATCA |
| | | | CAC |
| IL1RN | SEQ ID NO: 56 | PCR | ACAAGTTCTGGGGGACACAG |
| rs419598 | SEQ ID NO: 57 | PCR | AGGCCATGCTGCTGCAGACA |
| | SEQ ID NO: 58 | SBE | GACCTTCTATCTGAGGAACAACCA |
| | | | ACTAGTTGC |
| IL1RN | SEQ ID NO: 59 | PCR | TGAGCAAATGTGGCTCCTGGGGGT |
| rs9005 | | | TCT |
| | SEQ ID NO: 60 | PCR | CCCAAAGCCTGTCAAGGCCAAGGA |
| | | | CAT |
| | SEQ ID NO: 61 | SBE | GATGGCTGTGCCTCTGCCTGTCTCC |
| | | | CCCACC |
| IL1RN | SEQ ID NO: 62 | PCR | CCTGGAGGCCCCAGCAGGTGATGT |
| rs1794066 | | | TTA |
| | SEQ ID NO: 63 | PCR | CCTGCAGGAGCAGCTACCCTTGGG |
| | | | AAC |
| | SEQ ID NO: 64 | SBE | GGCCAGCCTGACCTGGGACCTGTG |
| | | | CCTCACCTC |
| IL1RN | SEQ ID NO: 65 | PCR | CACCCCAAAACCCAGTGGCTTGAA |
| 380092 | | | ACA |
| | SEQ ID NO: 66 | PCR | TGCAGTCTCCTCTCACAGGGGGCTA |
| | | | GACT |
| | SEQ ID NO: 67 | SBE | GGGGTCACTTTGGAAGCTGCATTCA |
| | | | GCAGAGTGCC |
| IL1RN | SEQ ID NO: 68 | PCR | GGCATTACCTGCAGCAAGGGCCTG |
| rs4251961 | | | TGT |
| | SEQ ID NO: 69 | PCR | GCCGTGACATTGTCCACAAGGCCA |
| | | | GAT |
| | SEQ ID NO: 70 | SBE | GAGCCCTAAGTCTAAGATAGGGCA |
| | | | GATAGCA |

PCR = Polymerase Chain Reaction
SBE = Single Base Extension genotyping

The design of additional oligonucleotides for use in the amplification and detection of metabolic gene polymorphic alleles by the method of the invention is facilitated by the availability of both updated sequence information from human chromosome 4q28-q31—which contains the human FABP2 locus, and updated human polymorphism information available for this locus. Suitable primers for the detection of a human polymorphism in metabolic genes can be readily designed using this sequence information and standard techniques known in the art for the design and optimization of primers sequences. Optimal design of such primer sequences can be achieved, for example, by the use of commercially available primer selection programs such as Primer 2.1, Primer 3 or GeneFisher (See also, Nicklin M. H. J., Weith A. Duff G. W., “A Physical Map of the Region Encompassing the Human Interleukin-1α, interleukin-1β, and Interleukin-1 Receptor Antagonist Genes” Genomics 19: 382 (1995); Nothwang H. G., et al. “Molecular Cloning of the Interleukin-1 gene Cluster: Construction of an Integrated YAC/PAC Contig and a partial transcriptional Map in the Region of Chromosome 2q13” Genomics 41: 370 (1997); Clark, et al. (1986) Nucl. Acids. Res., 14:7897-7914 [published erratum appears in Nucleic Acids Res., 15:868 (1987) and the Genome Database (GDB) project).

In another aspect, the invention features kits for performing the above-described assays. According to some embodiments, the kits of the present invention may include a means for determining a subject's genotype with respect to one or more metabolic gene. The kit may also contain a nucleic acid sample collection means. The kit may also contain a control sample either positive or negative or a standard and/or an algorithmic device for assessing the results and additional reagents and components including: DNA amplification reagents, DNA polymerase, nucleic acid amplification reagents, restrictive enzymes, buffers, a nucleic acid sampling device, DNA purification device, deoxynucleotides, oligonucleotides (e.g. probes and primers) etc.

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radio-labels, enzymes, fluorescent compounds, streptavidin, avidin, biotin, magnetic moieties, metal binding moieties, antigen or antibody moieties, and the like.

As described above, the control may be a positive or negative control. Further, the control sample may contain the positive (or negative) products of the allele detection technique employed. For example, where the allele detection technique is PCR amplification, followed by size fractionation, the control sample may comprise DNA fragments of the appropriate size. Likewise, where the allele detection technique involves detection of a mutated protein, the control sample may comprise a sample of mutated protein. However, it is preferred that the control sample comprises the material to be tested. For example, the controls may be a sample of genomic DNA or a cloned portion of a metabolic gene. Preferably, however, the control sample is a highly purified sample of genomic DNA where the sample to be tested is genomic DNA.

The oligonucleotides present in said kit may be used for amplification of the region of interest or for direct allele specific oligonucleotide (ASO) hybridization to the markers in question. Thus, the oligonucleotides may either flank the marker of interest (as required for PCR amplification) or directly overlap the marker (as in ASO hybridization).

Information obtained using the assays and kits described herein (alone or in conjunction with information on another genetic defect or environmental factor, which contributes to osteoarthritis) is useful for determining whether a non-symptomatic subject has or is likely to develop the particular disease or condition. In addition, the information can allow a more customized approach to preventing the onset or progression of the disease or condition. For example, this information can enable a clinician to more effectively prescribe a therapy that will address the molecular basis of the disease or condition.

The kit may, optionally, also include DNA sampling means. DNA sampling means are well known to one of skill in the art and can include, but not be limited to substrates, such as filter papers, the AmpliCard™ (University of Sheffield, Sheffield, England S10 2JF; Tarlow, J W, et al., J. of Invest. Dermatol. 103:387-389 (1994)) and the like; DNA purification reagents such as Nucleon™ kits, lysis buffers, proteinase solutions and the like; PCR reagents, such as 10× reaction buffers, thernostable polymerase, dNTPs, and the like; and allele detection means such as the HinfI restriction enzyme, allele specific oligonucleotides, degenerate oligonucleotide primers for nested PCR from dried blood.

### DEFINITIONS

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting. Other features and advantages of the invention will be apparent from the following detailed description and claims.

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth.

The term “allele” refers to the different sequence variants found at different polymorphic regions. The sequence variants may be single or multiple base changes, including without limitation insertions, deletions, or substitutions, or may be a variable number of sequence repeats.

The term “allelic pattern” refers to the identity of an allele or alleles at one or more polymorphic regions. For example, an allelic pattern may consist of a single allele at a polymorphic site, as for PPARG (+12) allele 1. Alternatively, an allelic pattern may consist of either a homozygous or heterozygous state at a single polymorphic site. For example, PPARG (+12) allele 2.2 is an allelic pattern in which there are two copies of the second allele and corresponds to the homozygous PPARG (+12) allele 2 state. Alternatively, an allelic pattern may consist of the identity of alleles at more than one polymorphic site.

The terms “control” or “control sample” refer to any sample appropriate to the detection technique employed. The control sample may contain the products of the allele detection technique employed or the material to be tested. Further, the controls may be positive or negative controls. By way of example, where the allele detection technique is PCR amplification, followed by size fractionation, the control sample may comprise DNA fragments of an appropriate size. Likewise, where the allele detection technique involves detection of a mutated protein, the control sample may comprise a sample of a mutant protein. However, it is preferred that the control sample comprises the material to be tested. For example, the controls may be a sample of genomic DNA or a cloned portion containing one or more metabolic genes. However, where the sample to be tested is genomic DNA, the control sample is preferably a highly purified sample of genomic DNA.

Body mass index (BMI) is a measure of body fat based on height and weight that applies to both men and women. BMI is considered in to fall into the so called “normal” range when BMI is between 18.5-24.9. According to this invention, an underweight subject has a BMI<18.5; an overweight subject in the range 25-29.9 and an obese subject has a BMI of 30-39.9, and BMI of 40 or greater is considered extremely obese.

The phrases “disruption of the gene” and “targeted disruption” or any similar phrase refers to the site specific interruption of a native DNA sequence so as to prevent expression of that gene in the cell as compared to the wild-type copy of the gene. The interruption may be caused by deletions, insertions or modifications to the gene, or any combination thereof.

The term “haplotype” as used herein is intended to refer to a set of alleles that are inherited together as a group (are in linkage disequilibrium) at statistically significant levels (P_{corr}<0.05). As used herein, the phrase “metabolic haplotype” refers to a haplotype of metabolic gene loci.

“Increased risk” refers to a statistically higher frequency of occurrence of the disease or condition in a subject carrying a particular polymorphic allele in comparison to the frequency of occurrence of the disease or condition in a member of a population that does not carry the particular polymorphic allele.

The term “isolated” as used herein with respect to nucleic acids, such as DNA or RNA, refers to molecules separated from other DNAs, or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

“Linkage disequilibrium” refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at expected frequencies are said to be in “linkage disequilibrium”. The cause of linkage disequilibrium is often unclear. It can be due to selection for certain allele combinations or to recent admixture of genetically heterogeneous populations. In addition, in the case of markers that are very tightly linked to a disease gene, an association of an allele (or group of linked alleles) with the disease gene is expected if the disease mutation occurred in the recent past, so that sufficient time has not elapsed for equilibrium to be achieved through recombination events in the specific chromosomal region. When referring to allelic patterns that are comprised of more than one allele, a first allelic pattern is in linkage disequilibrium with a second allelic pattern if all the alleles that comprise the first allelic pattern are in linkage disequilibrium with at least one of the alleles of the second allelic pattern.

The term “marker” refers to a sequence in the genome that is known to vary among subjects.

A “mutated gene” or “mutation” or “functional mutation” refers to an allelic form of a gene, which is capable of altering the phenotype of a subject having the mutated gene relative to a subject which does not have the mutated gene. The altered phenotype caused by a mutation can be corrected or compensated for by certain agents. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs (e.g. peptide nucleic acids) and as applicable to the embodiment being described, single (sense or antisense) and double-stranded polynucleotides.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A specific genetic sequence at a polymorphic region of a gene is an allele. A polymorphic region can be a single nucleotide, the identity of which differs in different alleles. A polymorphic region can also be several nucleotides long.

The term “propensity to disease,” also “predisposition” or “susceptibility” to disease or any similar phrase, means that certain alleles are hereby discovered to be associated with or predictive of a subject's incidence of developing a particular disease (e.g. a vascular disease). The alleles are thus over-represented in frequency in subjects with disease as compared to healthy subjects. Thus, these alleles can be used to predict disease even in pre-symptomatic or pre-diseased subjects.

As used herein, the term “specifically hybridizes” or “specifically detects” refers to the ability of a nucleic acid molecule to hybridize to at least approximately 6 consecutive nucleotides of a sample nucleic acid.

“Transcriptional regulatory sequence” is a generic term used throughout the specification to refer to DNA sequences, such as initiation signals, enhancers, and promoters, which induce or control transcription of protein coding sequences with which they are operably linked.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The term “risk-allele” refers to an allele of a gene which, when present in one or two copies in a subject results in increased propensity to a disorder, or phenotype under investigation. There can be several different risk-alleles, since several different nucleotide changes in a gene may affect the phenotype under study, with a variation in intensity. The term “risk-allele,” thus refers to an SNP or allele that is associated with high relative risk for a disorder or phenotype under investigation.

Dyslipidemia, as in this invention, is defined as the elevation of plasma cholesterol, triglycerides (TGs), or both, or a low high density lipoprotein (HDL) level that contributes to the development of atherosclerosis. A subject with dyslipidemia has lower level of HDL, about 40 mg/dL or lower for men, and 50 mg/dL or lower for women, or higher level of LDL, about 100 mg/dL or above, or higher level of triglycerides, about 150 mg/dL or above, or all.

According to some embodiments, lower level of HDL is 20-60 mg/dL or 50-59 mg/dL or 40-49 mg/dL or 30-39 mg/dL or <30 mg/dL; higher level of LDL is 100->190 mg/dL or 100-129 mg/dL or 130-159 mg/dL or 160-190 mg/dL or >190 mg/dL; and higher level of triglyceride is 150->500 mg/dL or 150-199 mg/dL or 200-500 mg/dL or >500 mg/dL.

The following examples are illustrative, but not limiting, of the methods and compositions of the present invention. Other suitable modifications and adaptations of the variety of conditions and parameters normally encountered in therapy and that are obvious to those skilled in the art are within the spirit and scope of the embodiments.

### EXAMPLES

### Example 1

### CALERIE (Comprehensive Assessment of the Long-Term Effects of Restricting Intake of Energy) Pilot Study

Chronic inflammation has been associated with metabolic syndrome and central obesity. The aim of this study was to investigate whether inflammatory genes such as the interleukin-1 (IL-1) cluster gene polymorphisms (SNPs) are associated with total body weight reduction, fat loss, and resting metabolic rate in response to two diets differing in carbohydrate content under caloric restriction. The genetic analysis of this study was performed retrospectively in the CALERIE (Comprehensive Assessment of the Long-Term Effects of Restricting Intake of Energy) pilot study population using samples from 29 healthy overweight (BMI; 27.8±1.6 kg/m2) adults who consented for genetic analysis. The CALERIE pilot study was a one-year randomized well-controlled trial in which high or low glycemic loads under calorie restricted diets were provided for the first six months of the trial, followed by dietary recommendation to adhere to a low calorie diet for the remaining 6 months of the study. Body weight, body fat mass, and resting metabolic rate were measured at baseline, 3, 6, 9 and 12 months.

Genotyping was performed for total of 14 SNPs in three inflammatory genes and these were analyzed in a linear regression model adjusting for age, gender and treatment group and using an additive genetic model. IL-1 receptor antagonist (IL1RN) gene SNPs, rs315952 (T*; responsive allele homozygote or carrier of the allele(*)), rs380092 (A*), rs4251961 (C*), and IL-1B gene SNPs IL-1B +3877 rs1143633 (G*) and IL-1B +6054(G*) showed statistical association (p 0.01-0.05) with percent change in body weight, at 3 months and 6 months from baseline. We also observed a strong association (p<0.05) between change in body fat mass and IL-1A +4845 (T*) and IL-1B +6054 (G*) at 3 months and 6 months from baseline. These results suggest that chronic inflammation may play an important role in maintaining an optimal body weight.

Detailed analysis was performed to identify the role of each allele on the measured outcomes. IL-1 cluster gene SNPs, IL1RN, rs315952, rs380092, rs4251961 and IL-1B +3877 rs1143633 (G*) and IL-1B +6054(G*) showed statistical association (p 0.01-0.05) with percent change in body weight, at 3 months and 6 months from baseline. A strong association (p<0.05) was also observed between change in body fat and IL-1A +4845 (T*) and IL-1B +6054 (G*), at 3 months and 6 months from baseline.

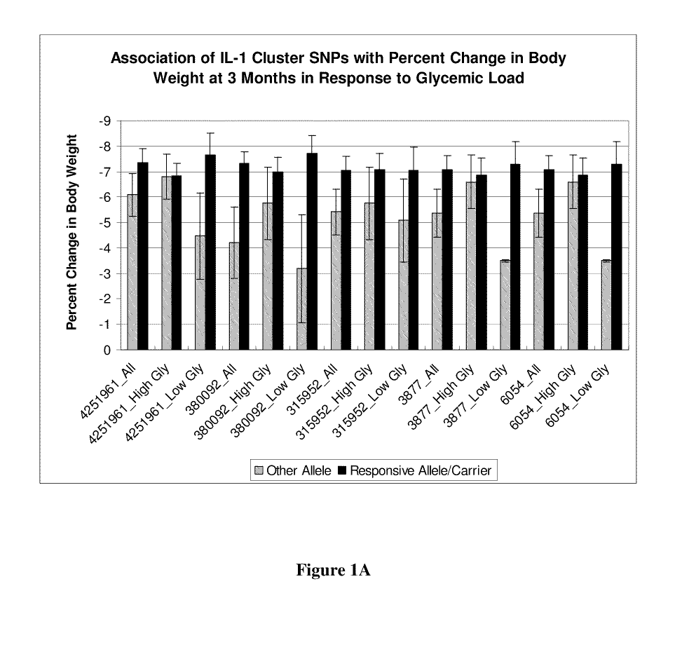

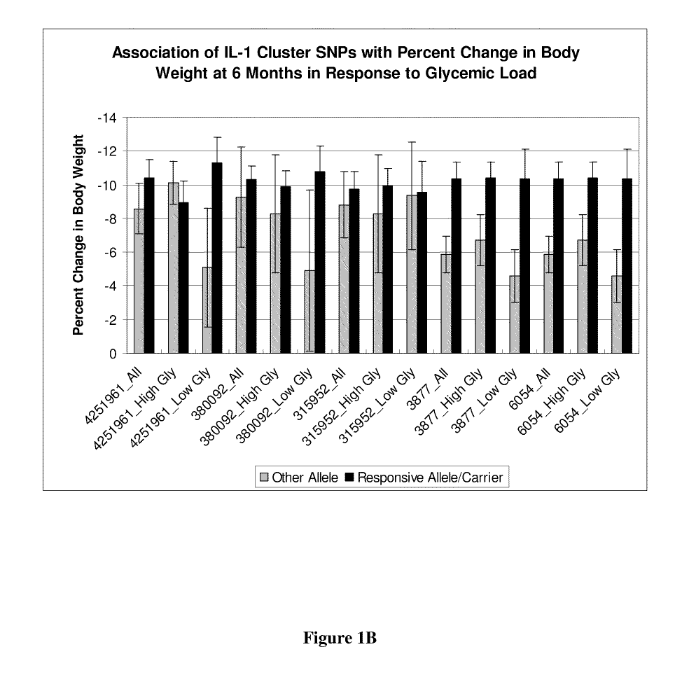

The table below provides data showing the effect on Total Body Weight (3 Months and 6 Months from Baseline) in response to different glycemic loads under caloric restriction. See also FIGS. 1A & B.

IL1RN, rs315952 (C/T) SNP: T allele was identified as responsive allele. T/T homozygotes or T carriers (T*) lose overall more percent body weight under calorie restriction and when prescribed low glycemic diet under calorie restriction.

IL1RN, rs380092 (A/T) SNP: A allele was identified as responsive allele. A/A homozygotes or A carriers (A*) lose overall more percent body weight under calorie restriction and when prescribed low glycemic diet under calorie restriction.

IL1RN, rs4251961 (C/T) SNP: C allele was identified as responsive allele. C/C homozygotes or C carriers (C*) lose overall more percent body weight under calorie restriction and when prescribed low glycemic diet under calorie restriction.

IL1B (+3877) rs1143633 (A/G) SNP: G allele was identified as responsive allele. G/G homozygotes or G carriers (G*) lose overall more percent body weight under calorie restriction and when prescribed low glycemic diet under calorie restriction.

IL1B +6054 (A/G) SNP: G allele was identified as responsive allele. G/G homozygotes or G carriers (G*) lose overall more percent body weight under calorie restriction and when prescribed low glycemic diet under calorie restriction.

TABLE 3

| | | Responsive | | | | | Responsive | | |
|---|---|---|---|---|---|---|---|---|---|
| SNP | Other Allele | Allele/Carrier | SNP/Diet Group | Other Allele | SD | SE | Allele/Carrier | SD | SE |
| | | | | 3 Months | | | | | |
| IL1RN, | T_T | C* | 4251961_All | −6.09 | 2.98 | 0.83 | −7.35 | 2.21 | 0.55 |
| rs4251961 | | | 4251961_High Gly | −6.81 | 2.66 | 0.89 | −6.82 | 1.21 | 0.49 |
| | | | 4251961_Low Gly | −4.47 | 3.41 | 1.70 | −7.67 | 2.65 | 0.84 |
| IL1RN, | T_T | A* | 380092_All | −4.21 | 3.12 | 1.39 | −7.32 | 2.21 | 0.45 |
| rs380092 | | | 380092_High Gly | −5.75 | 2.01 | 1.42 | −6.98 | 2.18 | 0.60 |
| | | | 380092_Low Gly | −3.19 | 3.67 | 2.12 | −7.73 | 2.27 | 0.69 |
| IL1RN, | C_C | T* | 315952_All | −5.42 | 1.81 | 0.91 | −7.05 | 2.73 | 0.56 |
| rs315952 | | | 315952_High Gly | −5.75 | 2.01 | 1.42 | −7.07 | 2.25 | 0.65 |
| | | | 315952_Low Gly | −5.08 | 2.32 | 1.64 | −7.04 | 3.24 | 0.93 |
| IL1B + 3877 | A_A | G* | 3877_All | −5.36 | 2.13 | 0.95 | −7.08 | 2.64 | 0.54 |
| | | | 3877_High Gly | −6.60 | 1.81 | 1.04 | −6.87 | 2.28 | 0.66 |
| | | | 3877_Low Gly | −3.50 | 0.08 | 0.05 | −7.30 | 3.05 | 0.88 |
| IL1B + 6054 | A_A | G* | 6054_All | −5.36 | 2.13 | 0.95 | −7.08 | 2.64 | 0.54 |
| | | | 6054_High Gly | −6.60 | 1.81 | 1.04 | −6.87 | 2.28 | 0.66 |
| | | | 6054_Low Gly | −3.50 | 0.08 | 0.05 | −7.30 | 3.05 | 0.88 |
| | | | | 6 Months | | | | | |
| IL1RN, | T_T | C* | 4251961_All | −8.58 | 5.30 | 1.47 | −10.42 | 4.28 | 1.07 |
| rs4251961 | | | 4251961_High Gly | −10.13 | 3.84 | 1.28 | −8.95 | 3.08 | 1.26 |
| | | | 4251961_Low Gly | −5.10 | 7.06 | 3.53 | −11.30 | 4.79 | 1.51 |
| IL1RN, | T_T | A* | 380092_All | −9.26 | 6.6 | 3.0 | −10.29 | 4.14 | 0.84 |
| rs380092 | | | 380092_High Gly | −8.28 | 4.92 | 3.48 | −9.87 | 3.44 | 0.95 |
| | | | 380092_Low Gly | −4.92 | 8.31 | 4.80 | −10.78 | 4.97 | 1.50 |
| IL1RN, | C_C | T* | 315952_All | −8.81 | 3.90 | 1.95 | −9.74 | 5.05 | 1.03 |
| rs315952 | | | 315952_High Gly | −8.28 | 4.92 | 3.48 | −9.93 | 3.58 | 1.03 |
| | | | 315952_Low Gly | −9.35 | 4.51 | 3.19 | −9.55 | 6.36 | 1.84 |
| IL1B + 3877 | A_A | G* | 3877_All | −5.87 | 2.46 | 1.10 | −10.37 | 4.79 | 0.98 |
| | | | 3877_High Gly | −6.71 | 2.65 | 1.53 | −10.39 | 3.35 | 0.97 |
| | | | 3877_Low Gly | −4.61 | 2.20 | 1.56 | −10.34 | 6.06 | 1.75 |
| IL1B + 6054 | A_A | G* | 6054_All | −5.87 | 2.46 | 1.10 | −10.37 | 4.79 | 0.98 |
| | | | 6054_High Gly | −6.71 | 2.65 | 1.53 | −10.39 | 3.35 | 0.97 |
| | | | 6054_Low Gly | −4.61 | 2.20 | 1.56 | −10.34 | 6.06 | 1.75 |

IL-1 gene cluster haplotype +4845 (T), +6054 (G), +3877 (G), +3954 (T), −511 (C), −3737 (C) shows strong association with weight loss in response to low glycemic diet under calorie restriction. SNPs that are in strong linkage disequilibrium (LD) in this region show strong association with the with weight loss in response to low glycemic diet under calorie restriction.

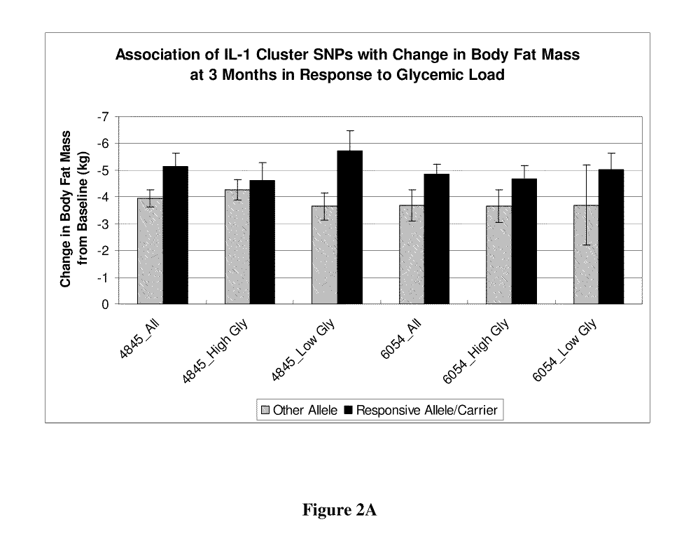

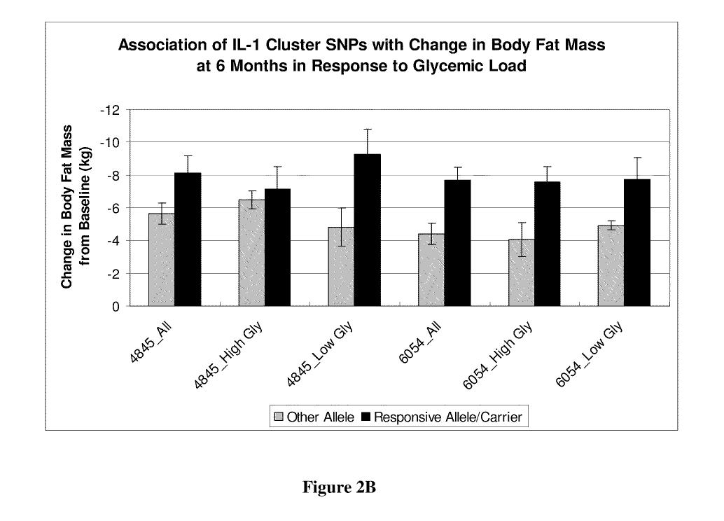

The table below provides data showing the effect on Change in body fat (3 & 6 Months from Baseline) in response to different glycemic loads under caloric restriction. See also FIGS. 2 A & B.

IL1A+4845 (G/T) SNP: T allele was identified as responsive allele. T/T homozygotes or T carriers (T*) lose overall more body fat under calorie restriction and when prescribed low glycemic diet under calorie restriction.

TABLE 4

| | | Responsive | | | | | Responsive | | |
|---|---|---|---|---|---|---|---|---|---|
| SNP | Other Allele | Allele/Carrier | SNP/Diet Group | Other Allele | SD | SE | Allele/Carrier | SD | SE |
| | | | | 3 Months | | | | | |
| IL1A + 4845 | G/G | T* | 4845_All | −3.96 | 1.09 | 0.31 | −5.14 | 2.10 | 0.51 |
| | | | 4845_High Gly | −4.27 | 0.93 | 0.38 | −4.60 | 2.05 | 0.68 |
| | | | 4845_Low Gly | −3.65 | 1.23 | 0.50 | −5.73 | 2.13 | 0.75 |
| IL1B + 6054 | A_A | G* | 6054_All | −3.68 | 1.30 | 0.58 | −4.85 | 1.88 | 0.38 |
| | | | 6054_High Gly | −3.66 | 1.06 | 0.61 | −4.67 | 1.75 | 0.50 |
| | | | 6054_Low Gly | −3.70 | 2.12 | 1.50 | −5.03 | 2.06 | 0.59 |
| | | | | 6 Months | | | | | |
| IL1A + 4845 | G/G | T* | 4845_All | −5.66 | 2.29 | 0.66 | −8.12 | 4.36 | 1.06 |
| | | | 4845_High Gly | −6.49 | 1.31 | 0.53 | −7.12 | 4.27 | 1.42 |
| | | | 4845_Low Gly | −4.82 | 2.85 | 1.16 | −9.25 | 4.46 | 1.58 |
| IL1B + 6054 | A_A | G* | 6054_All | −4.41 | 1.40 | 0.62 | −7.66 | 3.92 | 0.80 |
| | | | 6054_High Gly | −4.07 | 1.84 | 1.06 | −7.57 | 3.30 | 0.95 |
| | | | 6054_Low Gly | −4.93 | 0.39 | 0.28 | −7.76 | 4.61 | 1.33 |

IL1B + 6054 (G/A) SNP: G allele was identified as responsive allele Responsive allele G/G homozygotes or G carriers (G*) lose overall more body fat under calorie restriction and when prescribed low glycemic diet under calorie restriction.
IL-1 gene cluster haplotype +4845 (T), +6054 (G), +3877 (G), +3954 (T), −511 (C), −3737 (C) shows strong association with total body fat loss in response to low glycemic diet under calorie restriction. SNPs that are in strong linkage disequilibrium (LD) in this region show strong association with the with total body fat loss in response to low glycemic diet under calorie restriction.

While the invention has been described with reference to particularly preferred embodiments and examples, those skilled in the art recognize that various modifications may be made to the invention without departing from the spirit and scope thereof.

All of the above U.S. patents, U.S. patent application publications, U.S. patent applications, foreign patents, foreign patent applications and non-patent publications referred to in this specification and/or listed in the Application Data Sheet are incorporated herein by reference, in their entirety.

### Example 2

### Geisinger Study

In support of the CALORIE study, a second larger study was designed to investigate the association of the SNPs listed in Table 5 with resistance to weight loss in subjects on an energy restricted diet, either subjectly or in combination. Also, to investigate if any metabolic syndrome parameters, such as dyslipidemia or abnormal fasting glucose, are associated with these variations.

TABLE 5

| SNP | Base Change | rs number |
|---|---|---|
| FABP2(A54T) | (G/A) | 1799883 |
| PPARG(P12A) | (C/G) | 1801282 |
| ADRB2(Q27E) | (C/G) | 1042714 |
| ADRB2(R16G) | (A/G) | 1042713 |
| ADRB3(R64W) | (C/T) | rs4994 |
| MCR-4 | G/A | 2229616 |
| | A/G | 12970134 |
| MCR-4 | G/T | 477181 |
| | A/C | 502933 |
| | A/G | 4450508 |

IL-1 SNPs

| | | |
|---|---|---|
| IL1A(+4845) | (G/T) | 17561 |
| IL1B(−511) | (C/T) | 16944 |
| IL1B(−3737) | (T/C) | 4848306 |
| ILRN(+2018) | (T/C) | 419598 |
| ILRN(315952) | (C/T) | 315952 |
| ILRN(9005) | (A/G) | 9005 |
| IL1B(−1468) | (G/C) | 1143623 |
| IL1B(+3954) | (C/T) | 1143634 |

Study Design

The Geisinger Study was performed into two main stages. In Stage 1 (˜4 months), all the enrolled subjects were recommended a diet consisting of 1200-1500 kcal and 1500-1800 kcal, for Women and Men, respectively. Subject who lost >3% weight were classified as group A. In Stage 2 (˜4 months) all the subjects that lost <3% weight in Stage 1, were recommended a liquid diet of 1000 kcal and 1200 kcal, for Women and Men, respectively. Once on liquid diet, subjects who lost >5% of total body weight early on were classified as Group B (Early responders) and people who lost the same weight but at a later stage were put in Group C (Late responders). Subjects, who did not respond to either of the diets, were classified as Group D (Non-responders).

Body weight, lipid profile and other metabolic parameters were measured for all the enrolled subjects during their site visits.

Cases and Controls

824 subjects were evaluated at baseline. 372 subjects responded to low calorie diet with 4 weeks, and were classified in Group A, whereas 93 were classified in Group B (early responders of liquid diet for 120 days), and additional 92 were in Group C (late responders of liquid diet for 120 days). 267 subjects did not respond, i.e., lost less than 5% of body weight after being on liquid calorie diet for 120 days, and were classified in D category (controls).

In this study, a subject was classified as weight loss “resistant” based on a failure to lose 3% of their baseline bodyweight on dietary modification counseling designed to reduce caloric intake by 500 kcal, and if unsuccessful with diet modification, failure to lose the 5% on a prescription 1000 kcal liquid diet.

The weight resistant group will be considered as the Cases and the groups that lost weight will be considered as the Controls. Controls will be divided into two groups: (a) Control group-1 (Group A): those who lost weight on the recommended diet of 500 kcal deficit from estimated calories consumed daily and, (b) Control group-2 (Group BC): those who were initially resistant to weight loss with the above dietary plan for the first 4 months but eventually achieved weight loss when put on the 1000-1200 kcal liquid diet.

Sample Collection and Statistical Parameters

DNA samples obtained from the subjects were genotyped for all the SNPs listed in Table 5. Genetic association of these SNPs with body weight loss in low calorie diet responders versus non-responders was analyzed by logistical regression analysis in group-wise comparisons, adjusting the data for age, gender, antidepressant and diabetic medications, statins and diuretics. Genetic association was also analyzed for the lipid profile and metabolic parameters (Quantitative Traits) in a linear regression analysis using additive, dominant and recessive genetic models and adjusting for age, gender, metabolic score (co-morbidities), metformin, statins, anti-depression and diabetic medications. Data analysis was performed for three categories, Full data and two age stratified groups, Young Age (<47.5 year old) and Old Age (>47.5 year old).

Hardy Weinberg Equilibrium (HWE), linkage Disequilibrium (LD), and haplotype frequencies were determined by Haploview version 3.32, and subject-specific haplotypes were estimated using the HapAnalyzer program based on the EM algorithm. The χ² test was used to determine whether subject variants were in HWE at each locus.

Statistical analyses were performed with SPSS version 12.0 for Windows (Statistical Package for the Social Science, SPSS Ins., Chicago, Ill., USA). Differences in general characteristics between controls and cases were tested by independent t-test (continuous variables) or χ² test (categorical variables). Genotype distributions and allele frequencies were compared between controls and cases by χ² test or Fisher's exact test. The association between vertebral fracture and genotype or haplotype was calculated using the odds ratio (OR) [95% confidence intervals (CIs)] of a χ² test and a logistic regression analysis with adjustment for age, age at menopause, BMI, alcohol consumption, and log-BMD. To compare the differences in biomarkers according to genotype or haplotype either in control subjects or cases, we performed an independent t-test, Mann-Whitney non-parameteric test, one-way ANOVA or general linear model test followed by Bonferroni method with adjustment of covariates. We initially determined whether each variable presented a normal distribution before statistical testing, and then performed logarithmic transformation on the skewed variables (triglycerides, BMD, PICP, CTx, Lipoprotein(a)). For descriptive purposes, mean values are presented using untransformed values. Results are expressed as mean±S.E. A two-tailed value of P<0.05 was considered statistically significant.

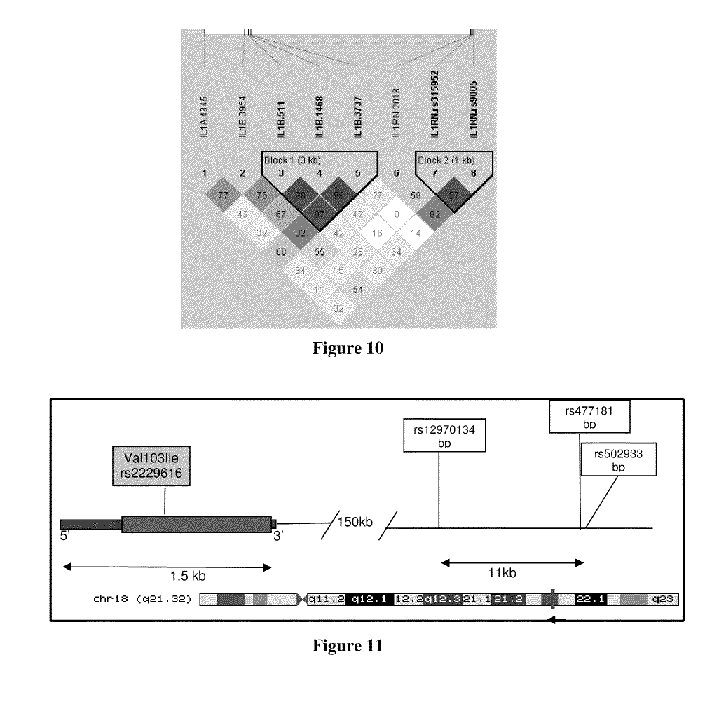

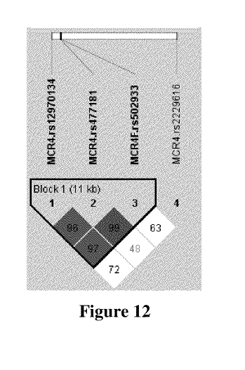

Linkage disequilibrium (LD) plot. Linkage disequilibrium (LD) plot were generated in Haploview software (r² shown) for all SNPs. See FIGS. 4, 10, and 12.

Demographics information about study subjects is shown in the table below, Table 6.

TABLE 6

| Characterisristic | Group A | | Group B | | Group C | | Group D | | p-value |
|---|---|---|---|---|---|---|---|---|---|
| Number of Patients | 372 | | 93 | | 92 | | 267 | | |
| Initial mean BMI (SD) | 51.0 | (8.3) | 49.6 | (7.8) | 49.9 | (8.5) | 48.4 | (7.2) | 0.000930* |
| Mean Age, yrs | 47.5 | (11.2) | 45.6 | (9.8) | 47.5 | (6.7) | 43.8 | (10.6) | 0.000235* |
| Males (#) | 68 | (18.3%) | 25 | (27.9%) | 23 | (25%) | 54 | (20.2%) | 0.205734 |
| Females (#) | 304 | (81.7%) | 68 | (73.3%) | 69 | (75%) | 213 | (79.8%) | |
| Age > 47.5 (#) | 200 | (53.8%) | 38 | (40.9%) | 49 | (53.3%) | 94 | (35.2%) | 0.00002 |
| Age < 47.5 (#) | 172 | (46.2%) | 55 | (59.1%) | 43 | (46.7%) | 173 | (64.8%) | |

| | | | | | |
|---|---|---|---|---|---|
| Hypertension | 174 | 43 | 40 | 117 | 0.869549 |
| Diabetes | 132 | 35 | 47 | 83 | 0.007278 |
| Hypercholesterolemia | 137 | 38 | 37 | 99 | 0.847789 |
| General symptoms | 124 | 29 | 20 | 91 | 0.150327 |
| Depressive disorder | 83 | 25 | 25 | 77 | 0.285318 |
| Oesophageal disease | 95 | 21 | 22 | 73 | |
| Osteoarthritis | 95 | 19 | 18 | 50 | 0.185914 |
| Asthma | 45 | 12 | 11 | 33 | 0.996596 |
| Family history of diabetes | 17 | 8 | 1 | 28 | 0.002569 |
| Affective psychoses | 14 | 4 | 0 | 10 | 0.293739 |

The table (Table 7) below provides data showing association of SNPs in ADRB2, ADRB3, IL1A, IL1B and IL1RN genes with weight loss for the responders versus non-responders under low calorie diet.

TABLE 7

| | | | | | | | | | | | |
|---|---|---|---|---|---|---|---|---|---|---|---|
| | | | | | | | | | | Geno. | |
| | Comparison | Assoc | Freq. | Freq. | OR | L95 | U95 | p-Value | p-Value | Counts | Geno. Counts |
| SNP | Group | Allele | Affected | Unaffected | (Adj) | (Adj) | (Adj) | (Adj) | (Perm) | (Affected) | (Unaffected) |

A_BC (low calorie diet responders vs liquid diet responders)

| | | | | | | | | | | | |
|---|---|---|---|---|---|---|---|---|---|---|---|
| IL1B.1468 | Full Data | C | 0.3243 | 0.2406 | 1.572 | 1.168 | 2.115 | 0.00286 | 0.0018 | 120\250 | 179\565 |
| IL1B.3737 | Full Data | T | 0.4027 | 0.4772 | 0.707 | 0.5385 | 0.9284 | 0.0126 | 0.013 | 149\221 | 355\389 |
| IL1B.511 | Full Data | T | 0.3757 | 0.3078 | 1.433 | 1.076 | 1.908 | 0.01371 | 0.0132 | 139\231 | 229\515 |
| IL1B.1468 | Old Age | C | 0.3506 | 0.2575 | 1.67 | 1.087 | 2.564 | 0.01915 | 0.0187 | 61\113 | 103\297 |
| IL1B.3737 | Young Age | T | 0.4133 | 0.5058 | 0.6691 | 0.4547 | 0.9846 | 0.04146 | 0.0442 | 81\115 | 174\170 |
| IL1B.511 | Young Age | T/T | 0.11224 | 0.03488372 | 3.79 | 1.324 | 10.85 | 0.01306 | 0.0104 | 11\87 | 6\166 |

ABC_D (responders vs resistant)

| | | | | | | | | | | | |
|---|---|---|---|---|---|---|---|---|---|---|---|
| IL1RN.rs315952 | Full data | C | 0.324 | 0.2774 | 1.263 | 1.006 | 1.586 | 0.04422 | 0.0477 | 173\361 | 309\805 |
| ADRB2.rs1042713 | Old Age | A/A | 0.07447 | 0.16376307 | 0.4244 | 0.1835 | 0.9818 | 0.04519 | 0.0341 | 7\87 | 47\240 |
| IL1A.4845 | Old Age | T | 0.3245 | 0.2526 | 1.501 | 1.017 | 2.214 | 0.04083 | 0.042 | 61\127 | 145\429 |
| IL1RN.rs315952 | Young Age | C | 0.3468 | 0.2722 | 1.406 | 1.047 | 1.887 | 0.02358 | 0.0241 | 120\226 | 147\393 |

BC_D (liquid diet responders vs resistant)

| | | | | | | | | | | | |
|---|---|---|---|---|---|---|---|---|---|---|---|
| ADRB3.rs4994 | Full data | C | 0.06367 | 0.0973 | 0.5896 | 0.3562 | 0.9759 | 0.03988 | 0.0419 | 34\500 | 36\334 |
| IL1B.1468 | Full data | C | 0.2659 | 0.3243 | 0.7314 | 0.5396 | 0.9913 | 0.0438 | 0.0451 | 142\392 | 120\250 |
| IL1RN.rs315952 | Full data | C/* | 0.53933 | 0.44864865 | 1.524 | 1.035 | 2.243 | 0.03288 | 0.0364 | 144\123 | 83\102 |
| IL1RN.rs315952 | Young Age | C/* | 0.56069 | 0.40816327 | 2.036 | 1.201 | 3.451 | 0.00831 | 0.0079 | 97\76 | 40\58 |

A_D (low calorie diet responders vs resistant)

| | | | | | | | | | | | |
|---|---|---|---|---|---|---|---|---|---|---|---|
| ADRB2.rs1042713 | Old Age | A/A | 0.07447 | 0.16 | 0.4151 | 0.1733 | 0.9942 | 0.04849 | 0.0332 | 7\87 | 32\168 |
| IL1A.4845 | Old Age | T | 0.3245 | 0.25 | 1.553 | 1.02 | 2.365 | 0.03996 | 0.0392 | 61\127 | 100\300 |

Logistical regression analysis of the group-wise comparison of responders versus non-responders to weight loss in response to low calorie diet showed strong association with SNPs in ADRB2 (R16G; rs1042713), ADRB3(rs4994), IL1A(rs17561), IL1B(rs16944) and IL1RN (rs315952) genes.

In the low calorie diet responders versus low calorie liquid diet responders comparison (A vs BC): IL1B gene SNPs, rs4848306 (−3737; C), rs1143623 (−1468; C) and rs16944 (−511; T) (p=0.002-0.05) were identified as resistant alleles. Subject with these genotypes showed resistance to weight loss in all three data sets (Full data, old age and young age) in response to calorie restriction. Subjects with IL1B gene SNPs, rs4848306 (−3737; T), rs1143623 (−1468; G) and rs16944 (−511; C) were identified as responsive alleles.

In the responders versus non-responders comparison (ABC vs D): subjects with ADRB2 SNP rs1042713 (G/*); IL1A SNP, rs17561 (+4845; T)) (p=0.04) and IL1RN SNP, rs315952 (C) (p=0.02-0.04) alleles, showed resistance to weight loss. Subjects with ADRB2 SNP rs1042713 (A/A) p=0.04); IL1A SNP, rs17561 (+4845; G) and IL1RN SNP, rs315952 (T) alleles showed response to weight loss.

In the low calorie liquid diet responders versus resistant group comparison (BC vs D): subjects with ADRB3 SNP rs4994 (T), IL1B SNP, rs1143623 (−1468; G) and IL1RN SNP, rs315952 (C/*) alleles, showed resistance to weight loss under calorie restriction. Subjects with ADRB3 SNP rs4994 (C) p=0.04), IL1B SNP, rs1143623 (−1468; C); p=0.043) and IL1RN SNP, rs315952 (T) alleles, showed response to weight loss under calorie restriction.

In the low calorie diet responders versus resistant group comparison (A vs D): ADRB2 SNP, rs1042713 (G/*) and IL1A SNP, rs17561 (+4845; T) (p=0.04) alleles showed resistance to weight loss under calorie restriction. ADRB2 SNP, rs1042713 (A/A); p=0.048) and IL1A SNP, rs17561 (+4845; G) alleles showed response to weight loss under calorie restriction.

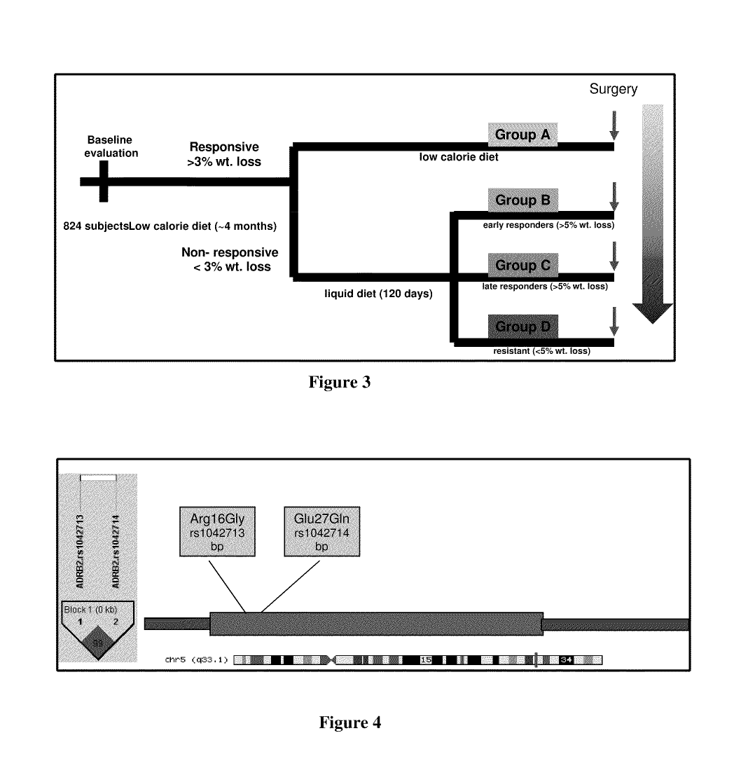

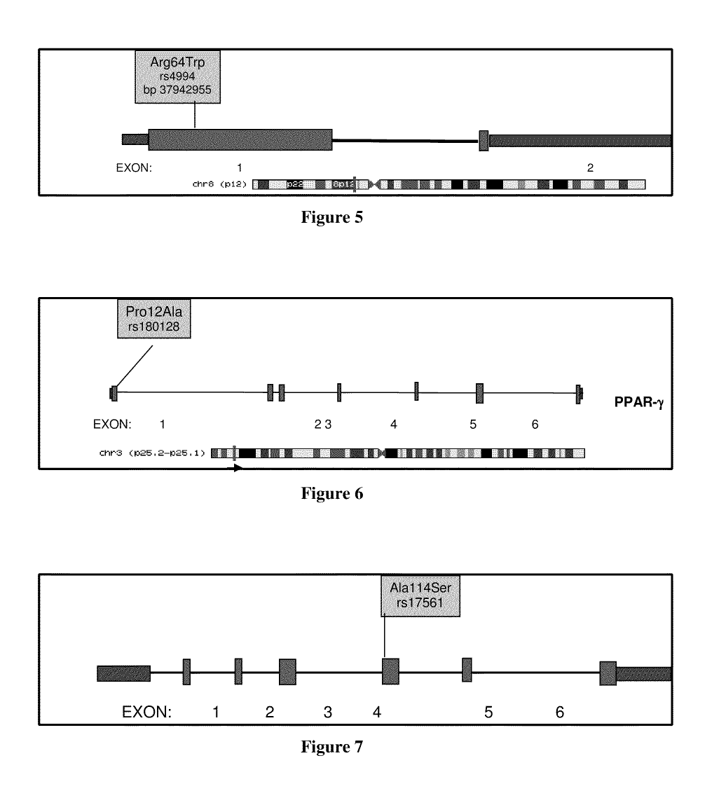

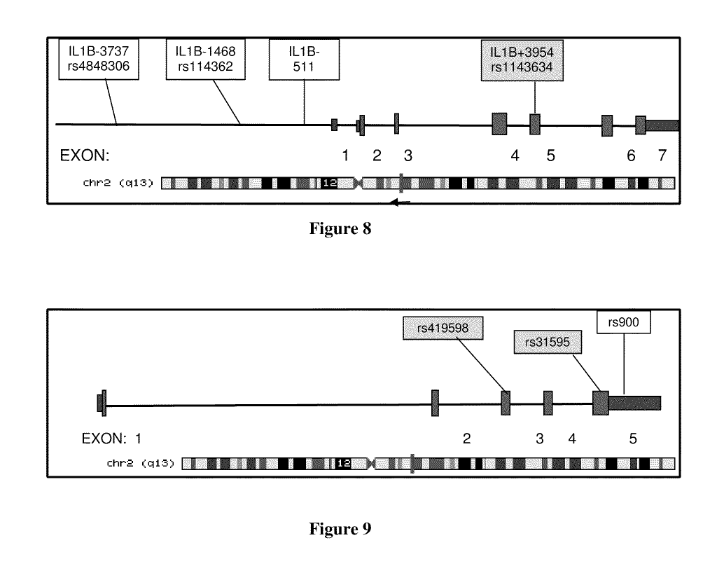

Subject SNPs position on the corresponding genes and their LD analysis is shown in the figures below (FIGS. 4-12).

Logistical regression analysis of the haplotypes in the responders versus non-responders group comparisons under calorie restriction showed a statistically significant association with different haplotype patterns in the IL1B and IL1RN genes. The associated haplotypes are shown in Table 8.

TABLE 8

| | | | | | | | | | | | |
|---|---|---|---|---|---|---|---|---|---|---|---|
| Gene | Haplotype | Cases | Controls | FreqCa | FreqCo | OR | OR (adj) | 95% LCI | 95% UCI | p-value | SNPs |

A_BC (Low calorie diet responders vs low calorie liquid diet responders)

| | | | | | | | | | | | |
|---|---|---|---|---|---|---|---|---|---|---|---|
| IL1B | CGT | 147.7 | 351.2 | 0.399 | 0.472 | 0.743 | Ref | | | | 511/1468/3737 |
| | TCC | 117.9 | 176.9 | 0.319 | 0.238 | 1.5 | 1.59356 | 1.17134 | 2.16797 | 0.00301 | 511/1468/3737 |
| | CCGT | 142.9 | 334.9 | 0.386 | 0.45 | 0.768 | Ref | | | | 3954/511/1468/3737 |
| | CTCC | 105.7 | 165.2 | 0.286 | 0.222 | 1.401 | 1.49505 | 1.08555 | 2.05902 | 0.01379 | 3954/511/1468/3737 |

A_D (Low calorie diet responders vs resistant)

| | | | | | | | | | | | |
|---|---|---|---|---|---|---|---|---|---|---|---|
| IL1RN | TG | 199.6 | 325.6 | 0.374 | 0.438 | 0.767 | Ref | | | | 315952/9005 |
| | CG | 172.4 | 208.4 | 0.323 | 0.28 | 1.225 | 1.35123 | 1.02379 | 1.78339 | 0.0335 | 315952/9005 |
| | TTG | 197.6 | 319.7 | 0.37 | 0.43 | 0.779 | Ref | | | | 2018/315952/9005 |
| | TCG | 160.2 | 184.8 | 0.3 | 0.248 | 1.297 | 1.3928 | 1.04691 | 1.85297 | 0.02292 | 2018/315952/9005 |

ABC_D (Responders vs resistant)

| | | | | | | | | | | | |
|---|---|---|---|---|---|---|---|---|---|---|---|
| IL1RN | TG | 199.6 | 478.9 | 0.374 | 0.43 | 0.792 | Ref | | | | 315952/9005 |
| | CG | 172.4 | 306.1 | 0.323 | 0.275 | 1.258 | 1.38107 | 1.06958 | 1.78326 | 0.01329 | 315952/9005 |
| | TTG | 197.6 | 468.4 | 0.37 | 0.421 | 0.809 | Ref | | | | 2018/315952/9005 |
| | TCG | 160.2 | 275.3 | 0.3 | 0.247 | 1.305 | 1.40227 | 1.07875 | 1.82282 | 0.01152 | 2018/315952/9005 |

ABC_D (Responders vs resistant) Young Age Group

| | | | | | | | | | | | |
|---|---|---|---|---|---|---|---|---|---|---|---|
| IL1RN | TG | 126.4 | 232.4 | 0.365 | 0.43 | 0.761 | Ref | | | | 315952/9005 |
| | CG | 119.6 | 145.6 | 0.346 | 0.27 | 1.432 | 1.51494 | 1.08693 | 2.11149 | 0.01421 | 315952/9005 |
| | TTG | 124.9 | 229.3 | 0.361 | 0.425 | 0.766 | Ref | | | | 2018/315952/9005 |
| | TCG | 112.3 | 131.6 | 0.325 | 0.244 | 1.491 | 1.56316 | 1.11291 | 2.19558 | 0.00996 | 2018/315952/9005 |

BC_D (Low calorie liquid diet responders vs resistant) Young Age Group

| | | | | | | | | | | | |
|---|---|---|---|---|---|---|---|---|---|---|---|
| IL1RN | TG | 126.4 | 82.1 | 0.365 | 0.419 | 0.798 | Ref | | | | 315952/9005 |
| | CG | 119.6 | 47.9 | 0.346 | 0.244 | 1.636 | 1.74862 | 1.10829 | 2.75891 | 0.01631 | 315952/9005 |
| | TTG | 124.9 | 80.8 | 0.361 | 0.412 | 0.805 | Ref | | | | 315952/9005 |
| | TCG | 112.3 | 43.8 | 0.325 | 0.223 | 1.67 | 1.74662 | 1.09482 | 2.78647 | 0.01928 | 2018/315952/9005 |

As shown in Table 8, two haplotype patterns consisting of 2 or 3 SNPs (rs315952/rs9005; CG) or (rs419598/rs315952/rs9005; TCG) in the IL1RN gene and 3 or 4 SNPs (rs16944/rs1143623/rs4848306; TCC) or (rs1143634/rs16944/rs1143623/rs4848306; CTCC) in the IL1B gene were associated with resistance to weight loss in response to low calorie diet (group comparison: A vs BC; A vs D; ABC vs D; BC vs D).

Serum Lipid Profiles

Blood fasting serum concentrations of total cholesterol and triglycerides were measured using an enzymatic method and commercially available kits on a Hitachi 7150 Autoanalyzer (Hitachi Ltd. Tokyo, Japan). After precipitation of serum chylomicron, low-density lipoprotein (LDL), and very low-density lipoprotein (VLDL) using dextran sulfate magnesium, the remaining high-density lipoprotein (HDL) cholesterol from the supernatant was measured by an enzymatic method. LDL cholesterol was indirectly estimated in subjects with serum triglyceride concentrations <400 mg/dl using the Friedewald formula. Methods for detecting blood lipoprotein are well known in the art.

Linear regression analysis was performed to identify association between IL1B, ADRB2, and MCR4, SNPs and lipid profile. The results of associated SNPs with lower levels HDL are shown in Table 9.

TABLE 9

| | | | | | | | | |
|---|---|---|---|---|---|---|---|---|
| SNP | A1 | Freq (%) | TEST | N | BETA | P | Permuted P | EMP2 |

Full Data HDL

| | | | | | | | | |
|---|---|---|---|---|---|---|---|---|
| ADRB2.rs1042713 | A/* | 60 | DOM | 824 | −1.651 | 0.02758 | 0.0275 | 0.4136 |
| IL1B.511 | C | 67 | ADD | 824 | −1.089 | 0.06279 | 0.06219 | 0.6805 |
| MCR4.rs12970134 | G | 71 | ADD | 824 | −1.411 | 0.01286 | 0.0116 | 0.2089 |
| MCR4.rs477181 | G/* | 86.5 | REC | 824 | −2.761 | 0.01025 | 0.009499 | 0.1949 |
| MCR4F.rs502933 | C/* | 86.8 | REC | 824 | −2.838 | 0.00882 | 0.008099 | 0.1735 |

Old - HDL

| | | | | | | | | |
|---|---|---|---|---|---|---|---|---|
| ADRB2.rs1042713 | A/* | 60 | DOM | 381 | −3.405 | 0.002385 | 0.0028 | 0.0428 |
| IL1B.1468 | G/G | 50 | DOM | 381 | −2.687 | 0.01453 | 0.0145 | 0.2403 |

Young - HDL

| | | | | | | | | |
|---|---|---|---|---|---|---|---|---|
| MCR4.rs12970134 | G | 71 | ADD | 443 | −2.162 | 0.004453 | 0.005299 | 0.08459 |
| MCR4.rs2229616 | A | 2 | ADD | 443 | −6.941 | 0.01775 | 0.0155 | 0.2748 |
| MCR4.rs477181 | G | 64 | ADD | 443 | −1.606 | 0.02673 | 0.0253 | 0.3832 |
| MCR4F.rs502933 | C | 64 | ADD | 443 | 1.585 | 0.02974 | 0.0272 | 0.4175 |

ADRB2 (rs1042713; A/*), IL1B (rs16944; −511; C) and (rs1143623; −1468; G/G) and MCR4 (rs12970134; G), (rs477181; G/*), (rs502933; C/*) and (rs2229616; A) SNPs showed strong association with lower levels of HDL in the full and age stratified data sets.

The results of associated SNPs with higher levels LDL are shown in Table 10.

TABLE 10

| | | | | | | | | |
|---|---|---|---|---|---|---|---|---|
| SNP | Freq (%) | A1 | TEST | n | BETA | P | Permuted P | EMP2 |

Full Data LDL

| | | | | | | | | |
|---|---|---|---|---|---|---|---|---|
| PPARG | 26 | G/* | DOM | 798 | 5.146 | 0.05725 | 0.05909 | 06672 |

Young - LDL

| | | | | | | | | |
|---|---|---|---|---|---|---|---|---|
| ADRB2.rs1042713 | 15 | A/A | REC | 429 | 8.637 | 0.05345 | 0.05429 | 0.6122 |

Two SNPs, ADRB2 (rs1042713; A/A) and PPARG (rs1801282; G/*) showed strong association with higher levels of LDL in the full and young age group data sets.

The results of associated SNPs with higher levels triglycerides (TG) are shown in Table 11.

TABLE 11

| | | | | | | | |
|---|---|---|---|---|---|---|---|
| SNP | A1 | TEST | Freq(%) | BETA | P | Permuted P | EMP2 |

Full Data - LogTG

| | | | | | | | |
|---|---|---|---|---|---|---|---|
| IL1B.1468 | C/C | REC | 6 | 0.1558 | 0.02844 | 0.027 | 0.4322 |
| IL1B.3954 | C | ADD | 75 | 0.06889 | 0.01441 | 0.0159 | 0.2379 |
| MCR4.rs2229616 | G/* | DOM | 96.5 | 0.1883 | 0.04067 | 0.0399 | 0.5421 |

Old - LogTG

| | | | | | | | |
|---|---|---|---|---|---|---|---|
| IL1B.1468 | C/C | REC | 6.5 | 0.3191 | 0.00247 | 0.0024 | 0.0404 |
| IL1RN.2018 | C/C | REC | 6.5 | 0.254 | 0.01477 | 0.0138 | 0.221 |
| IL1RN.rs9005 | A | ADD | 30 | 0.09431 | 0.02446 | 0.0281 | 0.3645 |
| MCR4.rs12970134 | G/G | REC | 91.4 | 0.1982 | 0.03102 | 0.0318 | 0.4264 |
| MCR4.rs2229616 | G/* | DOM | 95.8 | 0.2576 | 0.04492 | 0.0441 | 0.5647 |

Young - LogTG

| | | | | | | | |
|---|---|---|---|---|---|---|---|
| IL1B.3954 | C | ADD | 76 | 0.07654 | 0.04146 | 0.0395 | 0.5359 |

IL1B (rs1143623 ; −1468; C/C) and (rs1143634; +3954; C) , IL1RN (rs419598; +2018; C/C) and (rs9005; A) and MCR4 gene (rs12970134; G/G) and (rs2229616; G/*) SNPs showed strong association with higher levels of TG in the full and age stratified data sets.

The results of associated haplotypes on the ADRB2 and MCR4 genes with dyslipidemia are shown in Tables 12.

As shown in Table 12, two haplotypes consisting of (rs12970134/rs477181/rs502933;GGC) and (rs12970134/rs477181/rs502933/rs2229616; GTAG) SNPs on MCR4 gene were associated with lower levels of HDL and higher levels of TG, respectively. Haplotype pattern consisting of (rs1042713/rs1042714; AC) SNPs on ADRB2 gene show statistically significant association with both lower levels of HDL as well as higher levels of TG.

TABLE 12

| | | | | | |
|---|---|---|---|---|---|
| Gene | HAPLOTYPE | Frequency | BETA | P | SNPS |

HDL (FULL)

| | | | | | |
|---|---|---|---|---|---|
| MCR-4 | ATA | 0.28 | Ref | | rs12970134\|rs477181\|rs502933 |
| | GGC | 0.635 | −1.48 | 0.012 | rs12970134\|rs477181\|rs502933 |

HDL (YOUNG)

| | | | | | |
|---|---|---|---|---|---|
| MCR-4 | ATA | 0.28 | Ref | | rs12970134\|rs477181\|rs502933 |
| | GGC | 0.635 | −2 | 0.01 | rs12970134\|rs477181\|rs502933 |

HDL (FULL)

| | | | | | |
|---|---|---|---|---|---|
| ADRB2 | AC | 0.3724 | −1.52 | 0.0337 | rs1042713\|rs1042714 |
| | GC | 0.2016 | Ref | | rs1042713\|rs1042714 |

HDL (OLD)

| | | | | | |
|---|---|---|---|---|---|
| ADRB2 | AC | 0.3724 | −3.26 | 0.0025 | rs1042713\|rs1042714 |
| | GC | 0.2016 | Ref | | rs1042713\|rs1042714 |

TG (FULL)

| | | | | | |
|---|---|---|---|---|---|
| MCR-4 | ATAG | 0.2785 | ref | | rs12970134\|rs477181\|rs502933\|rs2229616 |
| | GTAG | 0.07483 | 0.1091 | 0.0266 | rs12970134\|rs477181\|rs502933\|rs2229616 |

TG (YOUNG)

| | | | | | |
|---|---|---|---|---|---|
| MCR-4 | ATAG | 0.2785 | ref | | rs12970134\|rs477181\|rs502933\|rs2229616 |
| | GTAG | 0.07483 | 0.125 | 0.553 | rs12970134\|rs477181\|rs502933\|rs2229616 |

TG (OLD)

| | | | | | |
|---|---|---|---|---|---|
| MCR-4 | GGCA | 0.01536 | Ref | 0.0512 | rs12970134\|rs477181\|rs502933\|rs2229616 |
| | ATAG | 0.2785 | 0.0792 | 0.0512 | rs12970134\|rs477181\|rs502933\|rs2229616 |

TABLE 13
Alleles that are resistant to weight loss due to calorie restriction.

| Gene | SNP | Risk-Allele Genotype | Risk-Allele | Allele Code |
|---|---|---|---|---|
| IL-1B | rs4848306 | C (−3737) | 2 | 2.2 (C/C) |
| | rs1143623 | G (−1468) when | 1 | 1.1 (G/G) |
| | | comparing BC vs. D | | |
| | rs16944 | T (−511) | 2 | 2.2 |
| ADRB2 | rs1042713 | G | 2 | 2.2 (G/G) |
| IL1A | rs17561 | T (+4845) | 2 | 2.2 (T/T) |
| IL1RN | rs315952 | C | 1 | 1.1 (C/C) |
| ADRB3 | rs4994 | T | 2 | 2.2 (T/T) |

TABLE 14
Alleles that were responsive to calorie restriction.

| | | Responsive-Allele | | Allele |
|---|---|---|---|---|
| Gene | SNP | Genotype | Responsive-Allele | Code |
| IL-1B | rs4848306 | T (−3737) | 1 | 1.1 (T/T) |
| | rs1143623 | C (−1468) when | 2 | 2.2 (C/C) |
| | | comparing BC vs. D | | |
| | rs16944 | C (−511) | 1 | 1.1 (C/C) |
| ADRB2 | rs1042713 | A/A | 1 | 1.1 (A/A) |
| IL1A | rs17561 | G (+4845) | 1 | 1.1 (G/G) |
| IL1RN | rs315952 | T | 2 | 2.2 (T/T) |
| ADRB3 | rs4994 | C | 1 | 1.1 (C/C) |

TABLE 15
Haplotypes that were resistant to calorie restriction.

| Gene | SNP | Genotype (allele) |
|---|---|---|
| IL-1RN | rs315952/rs9005 | CG |
| | rs419598/rs315952/rs9005 | TCG |
| IL1B | rs16944/rs1143623/rs4848306 | TCC |
| | (−511, T)/(−1468, C)/(−3737, C) | |
| | rs1143634/rs16944/rs1143623/rs4848306 | CTCC |
| | (+3954, C)/(−511, T)/(−1468, C)/(−3737, C) | |

What is claimed is:

1. A method for selecting an appropriate therapeutic/dietary regimen or lifestyle recommendation for a subject comprising genotyping said subject at one or more loci selected from the group consisting of: IL-1B, IL-1A, IL-1RN, ADRB2, ADRB3, and MCR4, wherein the presence of one or more allele within said loci is predictive of said subject's predisposition to weight loss in response to low calorie diet, or liquid diet, or both.

2. The method of claim 1, wherein selecting an appropriate therapeutic/dietary regimen or lifestyle recommendation for said subject comprises genotyping said subject at the SNP rs4848306 of IL-1B marker −3737, wherein the presence of allele C indicates that said subject is resistant, and presence of allele T indicates that said subject is predisposed to respond to weight loss in response to a low calorie diet, or liquid diet, or both.

3. The method of claim 1, wherein selecting an appropriate therapeutic/dietary regimen or lifestyle recommendation for said subject comprises genotyping said subject at the SNP rs1143623 of marker −1468 of IL-1B, wherein the presence of allele G indicates that said subject is resistant, and presence of allele C indicates that said subject is predisposed to respond to weight loss in response to a low calorie diet, or liquid diet, or both.

4. The method of claim 3, wherein presence of homozygous G/G allele is predictive that said subject is predisposed to lower level of HDL in response to low calorie diet, or liquid diet, or both.

5. The method of claim 3, wherein presence of homozygous C/C allele is predictive that said subject is predisposed to higher level of triglyceride in response to low calorie diet, or liquid diet, or both.

6. The method of claim 1, wherein selecting an appropriate therapeutic/dietary regimen or lifestyle recommendation for said subject comprises genotyping said subject at the SNP rs16944 of IL-1B marker −511, wherein the presence of allele T indicates that said subject is resistant, and presence of allele C indicates that said subject is predisposed to respond to weight loss in response to a low calorie diet, or liquid diet, or both.

7. The method of claim 6, wherein presence of heterozygous allele C is predictive that said subject is predisposed to lower level of HDL in response to low calorie diet, or liquid diet, or both.

8. The method of claim 1, wherein selecting an appropriate therapeutic/dietary regimen or lifestyle recommendation for said subject comprises genotyping said subject at the SNP rs1042713 of ADRB2, wherein the presence of heterozygous allele G indicates that said subject is resistant, and presence of homozygous allele A indicates that said subject is predisposed to respond to weight loss in response to a low calorie diet, or liquid diet, or both.

9. The method of claim 8, wherein presence of heterozygous allele G, is predictive that said subject is predisposed to lower level of HDL in response to low calorie diet, or liquid diet, or both.

10. The method of claim 8, wherein said allele is homozygous allele A is predictive that said subject is predisposed to higher level of LDL in response to low calorie diet, or liquid diet, or both.

11. The method of claim 1, wherein selecting an appropriate therapeutic/dietary regimen or lifestyle recommendation for said subject comprises genotyping said subject at the SNP rs17561 of marker +4845 of IL-1A, wherein the presence of allele T indicates that said subject is resistant, and presence of allele G indicates that said subject is predisposed to respond to weight loss in response to a low calorie diet, or liquid diet, or both.

12. The method of claim 1, wherein selecting an appropriate therapeutic/dietary regimen or lifestyle recommendation for said subject comprises genotyping said subject at the SNP rs315952 of IL-1RN, wherein the presence of allele C indicates that said subject is resistant, and presence of allele T indicates that said subject is predisposed to respond to weight loss in response to a low calorie diet, or liquid diet, or both.

13. The method of claim 1, wherein selecting an appropriate therapeutic/dietary regimen or lifestyle recommendation for said subject comprises genotyping said subject at the SNP rs4994 of ADRB3, wherein the presence of allele T indicates that said subject is resistant, and presence of allele C indicates that said subject is predisposed to respond to weight loss in response to a low calorie diet, or liquid diet, or both.

14. The method of claim 1, wherein selecting an appropriate therapeutic/dietary regimen or lifestyle recommendation for said subject comprises detecting in said subject allele G at +6054 marker of IL-1B, wherein the presence of said allele indicates that said subject is predisposed to respond to weight loss in response to a low calorie diet, wherein low calorie diet is a low glycemic diet under calorie restriction.

15. The method of claim 1, wherein selecting an appropriate therapeutic/dietary regimen or lifestyle recommendation for said subject comprises detecting in said subject allele G at SNP rs1143633 of IL-1B, wherein the presence of said allele indicates that said subject is predisposed to respond to weight loss in response to a low calorie diet, wherein low calorie diet is a low glycemic diet under calorie restriction.

16. The method of claim 1, wherein selecting an appropriate therapeutic/dietary regimen or lifestyle recommendation for said subject comprises detecting in said subject allele A at SNP rs380092 of IL-1RN, wherein the presence of said allele indicates that said subject is predisposed to respond to weight loss in response to a low calorie diet, wherein low calorie diet is a low glycemic diet under calorie restriction.

17. The method of claim 1, wherein selecting an appropriate therapeutic/dietary regimen or lifestyle recommendation for said subject comprises detecting in said subject allele C at SNP rs4251961 of IL-1RN, wherein the presence of said allele indicates that said subject is predisposed to respond to weight loss in response to a low calorie diet, wherein low calorie diet is a low glycemic diet under calorie restriction.

18. A method of selecting an appropriate therapeutic/dietary regimen or lifestyle recommendations for a subject, comprising genotyping said subject for composite genotype at one or more loci selected from the group consisting of: IL-1B, IL-1A, IL-1RN, ADRB2, ADRB3, and MCR4, wherein the presence of one or more said composite genotypes within said loci is predictive of said subject's predisposition to weight loss in response to low calorie diet, or liquid diet, or both.

19. The method of claim 18, wherein selecting an appropriate therapeutic/dietary regimen or lifestyle recommendation for said subject comprises the steps of:
a) genotyping said subject at:
(i) SNP rs315952 of IL-1RN; and
(ii) SNP rs9005 of IL-1RN;
b) determining whether said subject has a composite genotype comprising the allelic pattern or haplotype of: heterozygous allele C at SNP rs315952 of IL-1RN and heterozygous allele G at rs9005 of IL-1RN; wherein the presence of said haplotype indicates that said subject is resistant to weight loss in response to a low calorie or liquid diet.

20. The method of claim 18, wherein selecting an appropriate therapeutic/dietary regimen or lifestyle recommendation for said subject comprises the steps of:
a) genotyping said subject at:
(i) SNP rs419598 of IL-1RN;
(ii) SNP rs315952 of IL-1RN; and
(iii) SNP rs9005 of IL-1RN;
b) determining whether said subject has a composite genotype comprising the allelic pattern or haplotype of: heterozygous allele T at SNP rs419598 of IL-1RN, heterozygous allele C at SNP rs315952 of IL-1RN, and heterozygous allele G at rs9005 of IL-1RN; wherein the presence of said haplotype indicates that said subject is resistant to weight loss in response to a low calorie or liquid diet.

21. The method of claim 18, wherein selecting an appropriate therapeutic/dietary regimen or lifestyle recommendation for said subject comprises the steps of:
a) genotyping said subject at:
(i) SNP rs16944 of IL-1B;
(ii) SNP rs1143623 of IL-1B; and
(iii) SNP rs4848306 of IL-1B;
b) determining whether said subject has a composite genotype comprising the allelic pattern or haplotype of: heterozygous allele T at SNP rs16944 of IL-1B, heterozygous allele C at SNP rs1143623 of IL-1B, and heterozygous allele C at SNP rs4848306 of IL-1B; wherein the presence of said haplotype indicates that said subject is resistant to weight loss in response to a low calorie or liquid diet.

22. The method of claim 18, wherein selecting an appropriate therapeutic/dietary regimen or lifestyle recommendation for said subject comprises the steps of:
a) genotyping said subject at:
(i) SNP rs1143634 of IL-1B
(ii) SNP rs16944 of IL-1B;
(iii) SNP rs1143623 of IL-1B; and
(iv) SNP rs4848306 of IL-1B;
b) determining whether said subject has a composite genotype comprising the allelic pattern or haplotype of: heterozygous allele C at SNP rs1143634 of IL-1B, heterozygous allele T at SNP rs16944 of IL-1B, heterozygous allele C at SNP rs1143623 of IL-1B, and heterozygous allele C at SNP rs4848306 of IL-1B; wherein the presence of said haplotype indicates that said subject is resistant to weight loss in response to a low calorie or liquid diet.

23. The method of claim 18, wherein selecting an appropriate therapeutic/dietary regimen or lifestyle recommendation for said subject comprises the steps of:
a) genotyping said subject at:
(i) SNP rs1042713 of ADRB2; and
(ii) SNP rs1042714 of ADRB2;
b) determining whether said subject has a composite genotype comprising the allelic pattern or haplotype of: heterozygous allele A at SNP rs 1042713 of ADRB2, and heterozygous allele C at SNP rs1042714 of ADRB2; wherein the presence of said haplotype is predictive that said subject is predisposed to lower level of HDL and higher level of triglyceride in response to a low calorie or liquid diet.

24. The method of claim 18, wherein selecting an appropriate therapeutic/dietary regimen or lifestyle recommendation for said subject comprises the steps of:
a) genotyping said subject at:
(i) SNP rs12970134 of MCR4;
(ii) SNP rs477181 of MCR4; and
(iii) SNP rs502933 of MCR4;
b) determining whether said subject has a composite genotype comprising the allelic pattern or haplotype of: heterozygous allele G at SNP rs12970134 of MCR4, heterozygous allele G at SNP rs477181 of MCR4, and heterozygous allele C at SNP rs502933 of MCR4; wherein the presence of said haplotype is predictive that said subject is predisposed to lower level of HDL in response to a low calorie or liquid diet.

25. The method of claim 18, wherein selecting an appropriate therapeutic/dietary regimen or lifestyle recommendation for said subject comprises the steps of:
a) genotyping said subject at:
(i) SNP rs12970134 of MCR4;
(ii) SNP rs477181 of MCR4;
(iii) SNP rs502933 of MCR4; and
(iv) SNP rs2229616 of MCR4;
b) determining whether said subject has a composite genotype comprising the allelic pattern or haplotype of: heterozygous allele G at SNP rs12970134 of MCR4, heterozygous allele T at SNP rs477181 of MCR4, heterozygous allele A at SNP rs502933 of MCR4, and heterozygous allele G at rs2229616 of MCR4; wherein the presence of said haplotype is predictive that said subject is predisposed to higher level of triglyceride in response to a low calorie or liquid diet.

26. A kit for determining a subject's response to low calorie or liquid diet toward achieving weight loss comprising reagents and instructions for genotyping said subject at one or more loci selected from the group consisting of: IL-1B, IL-1A, IL-1RN, ADRB2, ADRB3, and MCR4, wherein the presence of one or more risk allele within said locus is predictive of said subject's predisposition to weight loss in response to low calorie diet, or liquid diet, or both.

27. The kit according to claim 26, wherein determining said subject's response to low calorie or liquid diet toward achieving weight loss comprises reagents and instructions for detecting in said subject an allele at SNP rs4848306 of IL-1B marker −3737, wherein the reagents comprises primers, buffers, salts for detecting said allele.

28. The kit according to claim 26, wherein determining said subject's response to low calorie or liquid diet toward achieving weight loss comprises reagents and instructions for detecting in said subject an allele at SNP rs1143623 of IL-1B marker −1468, wherein the reagents comprises primers, buffers, salts for detecting said allele.

29. The kit according to claim 26, wherein determining said subject's response to low calorie or liquid diet toward achieving weight loss comprises reagents and instructions for detecting in said subject an allele at SNP rs16944 of IL-1B marker −511, wherein the reagents comprises primers, buffers, salts for detecting said allele.

30. The kit according to claim 26, wherein determining said subject's response to low calorie or liquid diet toward achieving weight loss comprises reagents and instructions for detecting in said subject an allele at SNP rs1042713 of ADRB2, wherein the reagents comprises primers, buffers, salts for detecting said allele.

31. The kit according to claim 26, wherein determining said subject's response to low calorie or liquid diet toward achieving weight loss comprises reagents and instructions for detecting in said subject an allele at SNP rs17561 of IL-1A marker +4845, wherein the reagents comprises primers, buffers, salts for detecting said allele.

32. The kit according to claim 26, wherein determining said subject's response to low calorie or liquid diet toward achieving weight loss comprises reagents and instructions for detecting in said subject an allele at SNP rs315952 of IL-1RN, wherein the reagents comprises primers, buffers, salts for detecting said allele.

33. The kit according to claim 26, wherein determining said subject's response to low calorie or liquid diet toward achieving weight loss comprises reagents and instructions for detecting in said subject an allele at SNP rs4994 of ADRB3, wherein the reagents comprises primers, buffers, salts for detecting said allele.

34. The kit according to claim 26, wherein determining said subject's response to low calorie or liquid diet toward achieving weight loss comprises reagents and instructions for detecting in said subject an allele G at +6054 marker of IL-1B, wherein the reagents comprises primers, buffers, salts for detecting said allele.

35. The kit according to claim 26, wherein determining said subject's response to low calorie or liquid diet toward achieving weight loss comprises reagents and instructions for detecting in said subject an allele G at SNP rs1143633 of IL-1B, wherein the reagents comprises primers, buffers, salts for detecting said allele.

36. The kit according to claim 26, wherein determining said subject's response to low calorie or liquid diet toward achieving weight loss comprises reagents and instructions for detecting in said subject an allele A at SNP rs380092 of IL-1RN, wherein the reagents comprises primers, buffers, salts for detecting said allele.

37. The kit according to claim 26, wherein determining said subject's response to low calorie or liquid diet toward achieving weight loss comprises reagents and instructions for detecting in said subject an allele at C at SNP rs4251961 of IL-1RN, wherein the reagents comprises primers, buffers, salts for detecting said allele.

38. A kit for determining a subject's response to low calorie or liquid diet toward achieving weight loss comprising genotyping said subject for composite genotype at one or more loci selected from the group consisting of: IL-1B, IL-1A, IL-1RN, ADRB2, ADRB3, and MCR4, wherein the presence of one or more risk allele within said locus is predictive of said subject's predisposition to weight loss in response to low calorie diet, or liquid diet, or both.

39. The kit according to claim 38, wherein determining said subject's composite genotype, comprises reagents and instructions for genotyping said subject at:
(i) SNP rs315952 of IL-1RN; and
(ii) SNP rs9005 of IL-1RN;
wherein the reagents comprises primers, buffers, salts for detecting said allele.

40. The kit according to claim 38, wherein determining said subject's composite genotype, comprising reagents and instructions for genotyping said subject at:
(i) SNP rs419598 of IL-1RN;
(ii) SNP rs315952 of IL-1RN; and
(iii) SNP rs9005 of IL-1RN;
wherein the reagents comprises primers, buffers, salts for detecting said allele.

41. The kit according to claim 38, wherein determining said subject's composite genotype, comprising reagents and instructions for genotyping said subject at:
(i) SNP rs16944 of IL-1B;
(ii) SNP rs1143623 of IL-1B; and
(iii) SNP rs4848306 of IL-1B;
wherein the reagents comprises primers, buffers, salts for detecting said allele.

42. The kit according to claim 38, wherein determining said subject's composite genotype, comprising reagents and instructions for genotyping said subject at:
(i) SNP rs1143634 of IL-1B
(ii) SNP rs16944 of IL-1B;
(iii) SNP rs1143623 of IL-1B; and
(iv) SNP rs4848306 of IL-1B;
wherein the reagents comprises primers, buffers, salts for detecting said allele.

43. The kit according to claim 38, wherein determining said subject's composite genotype, comprising reagents and instructions for genotyping said subject at:
(i) SNP rs1042713 of ADRB2; and
(ii) SNP rs1042714 of ADRB2;
b) determining whether said subject has a composite genotype comprising the allelic pattern or haplotype of: heterozygous allele A at SNP rs 1042713 of ADRB2, and heterozygous allele C at SNP rs1042714 of ADRB2; wherein the presence of said haplotype is predictive that said subject has lower level of HDL and higher level of triglyceride.

44. The kit according to claim 38, wherein determining said subject's composite genotype, comprising reagents and instructions for:
a) genotyping in said subject's DNA one or more of the following alleles, selected from the group consisting of:
(i) SNP rs12970134 of MCR4;
(ii) SNP rs477181 of MCR4; and
(iii) SNP rs502933 of MCR4;
b) determining whether said subject has a composite genotype comprising the allelic pattern or haplotype of: heterozygous allele G at SNP rs12970134 of MCR4, heterozygous allele G at SNP rs477181 of MCR4, and heterozygous allele C at SNP rs502933 of MCR4; wherein the presence of said haplotype is predictive that said subject has lower level of HDL.

45. The kit according to claim 38, wherein determining said subject's composite genotype, comprising reagents and instructions for:
a) genotyping in said subject's DNA one or more of the following alleles, selected from the group consisting of:
(i) SNP rs12970134 of MCR4;
(ii) SNP rs477181 of MCR4;
(iii) SNP rs502933 of MCR4; and
(iv) SNP rs2229616 of MCR4;
wherein the reagents comprises primers, buffers, salts for detecting said allele.
